(19) United States
(12) Patent Application Publication
(10) Pub. No.: US 2025/0243250 A1
(43) Pub. Date: Jul. 31, 2025

(54) THERAPEUTIC NANOCONJUGATES AND USES THEREOF

(71) Applicants: UNIVERSITAT AUTÒNOMA DE BARCELONA (UAB), Cerdanyola dei Vallés (ES); FUNDACIÓ INSTITUT DE RECERCA DE L'HOSPITAL DE LA SANTA CREU I SANT PAU, Barcelona (ES); CONSORCIO CENTRO DE INVESTIGACIÓN BIOMÉDICA EN RED, M.P., Madrid (ES)

(72) Inventors: Antonio Pedro VILLAVERDE CORRALES, Cerdanyola del Vallès (ES); Esther VÁZQUEZ GÓMEZ, Cerdanyola del Vallés (ES); Ugutz UNZUETA ELORZA, Barcelona (ES); Ramón MANGUES BAFALLUY, Barcelona (ES); María Virtudes CÉSPEDES NAVARRO, Madrid (ES); Isolda CASANOVA RIGAT, Madrid (ES)

(21) Appl. No.: 18/968,979

(22) Filed: Dec. 4, 2024

Related U.S. Application Data
(62) Division of application No. 16/631,004, filed on Jan. 14, 2020, now Pat. No. 12,202,866.

(30) Foreign Application Priority Data
Jul. 14, 2017 (EP) EP17382461.6

(51) Int. Cl. C07K 14/47; A61K 49/14; A61K 49/18

(52) U.S. Cl. CPC C07K 14/4702; A61K 49/146; A61K 49/1821; C07K 2319/30

(57) ABSTRACT

The present invention relates to nanostructured conjugates, more specifically to nanostructured fusion proteins suitable for the selective delivery of their conjugated therapeutic agents to specific cell and tissue types. It also relates to nanoparticles comprising such nanostructured proteins and the therapeutic uses thereof.

### CROSS-REFERENCE TO RELATED APPLICATIONS

This application is a divisional of U.S. application Ser. No. 16/631,004, which has a § 371 (c) date of Jan. 14, 2020, and issued as U.S. Pat. No. 12,202,866, on Jan. 21, 2025, which is a 35 U.S.C. § 371 National Phase Application of PCT/EP2018/069303 filed on Jul. 16, 2018, claiming priority to European Application No. 17382461.6, filed on Jul. 14, 2017, all of which are incorporated herein by reference in their entireties.

### REFERENCE TO SEQUENCE LISTING SUBMITTED ELECTRONICALLY

The content of the electronically submitted sequence listing in XML format (Name: 5489_0030002_Sequencelisting_ST26.xml; Size: 64,529 bytes; and Date of Creation: Apr. 15, 2025) filed with the application is incorporated herein by reference in its entirety.

### FIELD OF THE INVENTION

The present invention relates to the field of nanostructured protein materials, more specifically to therapeutic agent-carrying fusion proteins which can be used for therapy.

### BACKGROUND OF THE INVENTION

The systemic administration of drugs in form of nanoconjugates benefits from enhanced drug stability when compared to free molecules. Valuable additional properties such as cell targeting might be also merged into a given hybrid composite through the chemical incorporation of functional groups in nanoscale vehicles, taking profit from the high surface/volume ratio of nanomaterials. When administered systemically, the resulting drug loaded conjugates sizing between ˜8 and 100 nm escape from renal filtration in absence of aggregation in lung or other highly vascularized organs. This fact, combined with appropriate physicochemical properties of the material might result in extended circulation time and prolonged drug exposure to target organs, thus enhancing the therapeutic impact and benefits for the patient.

Among the diversity of materials under investigation as drug carriers, that includes metals, ceramics, polymers and carbon nanotubes, proteins offer unique properties regarding biocompatibility and degradability that, in the context of rising nanotoxicological concerns, make them especially appealing. As the engineering of protein self-assembling into nanostructured materials is rapidly progressing and the control over the final geometry and physicochemical properties becomes tighter, protein materials are gaining functional and structural versatility as vehicles from chemically coupled drugs.

In fact, the attachment of a cytotoxic “payload” to an antibody to form an antibody-drug conjugate (ADC) has been shown to provide a mechanism for selective delivery of the cytotoxic agent to cancer cells via the specific binding of the antibody to cancer-selective cell surface molecules. Multiple examples of this strategy have been proved to be effective, like gemtuzumab ozogamicin, which comprises an anti-CD33 antibody conjugated to a highly potent DNA-targeting antibiotic, calicheamicin, which was used against acute myeloid leukemia. Also, maytansinoids, a highly potent microtubule-disrupting agents, have been tested as payloads for ADCs, resulting in the formulation ado-trastuzumab emtansine for treating HER2-positive breast cancer.

Nonetheless, the structural complexity of antibodies may become a cumbersome hindrance in terms of cost and synthesis. The inventors previously probed into the field of nanomedicine by applying a nanoarchitectonic principle based on the addition, to a core protein, of a cationic N-terminal domain plus a C-terminal poly-histidine. [Serna, N. et al. 2016. Nanomedicine, 12:1241-51] It has been described in the art that these end-terminal tags and the resulting charge balance in the whole fusion promote self-assembling and oligomerization of monomeric proteins as robust toroid nanoparticles, stable in plasma [Cespedes, M. V. et al. 2014. ACS Nano., 8:4166-4176] and with high cellular penetrability if empowered with cell-targeting peptides. [Xu, Z. K. et al. 2015. Materials Letters, 154:140-3] The building blocks of these protein structures might also contain functional peptides such as cell-targeting agents, endosomolytic agents or nuclear localization signals, in form of fused stretches with modular organization.

Since current therapy methods still show a margin of failure, mostly due to tumor resistance phenomena which may result from intra-tumor clonal selection of those cells most resistant to the chemotherapy, for instance, there is still in the art a need for the development of more specific therapeutic approaches which can be targeted to the concrete tumor cells responsible for therapy failure and tumor progression while reducing the side and off-target effects of the therapeutic agents

### SUMMARY OF THE INVENTION

In a first aspect, the invention relates to a fusion protein comprising
(i) a polycationic peptide,
(ii) an intervening polypeptide region and
(iii) a positively charged amino acid-rich region,
wherein the intervening polypeptide region is conjugated to at least one therapeutic agent.

In further aspects, the invention relates to a method to prepare the fusion protein of the first aspect, to a method to prepare nanoparticles comprising multiple copies of the fusion protein according to the first aspect of the invention, to a nanoparticle comprising multiple copies of the fusion protein of the invention or a nanoparticle which has been obtained by the method of the invention to prepare nanoparticles.

The invention also pertains to a fusion protein or a nanoparticle according to the invention for use in medicine.

In a final aspect, the invention relates to a fusion protein or a nanoparticle according to the invention for use in the treatment of cancer.

### DESCRIPTION OF THE FIGURES

FIG. 1. Synthesis of thiol functionalized oligo-FdU and physicochemical characterization (a) Synthesis of Oligo-FdU functionalized with thiol: the pentamer oligo-(FdU) 5-SH (oligo-FdU) was synthesized in 1 μmol scale on an RNA/DNA synthesizer using β-cyanoethylphosphoramiditechemistry. 3′-Thiol-modifier C3 controlled pore glass (CPG) (Link Technologies) was used as solid support for the synthesis. First the hexaethyleneglycol (HEG) phosphoramidite (Glen Research) was incorporated. Then, the synthesis was completed by repetitive additions of the dimethoxytrityl (DMT)-protected-5-fluoro-2′-deoxyuridinephosphoramidite unit. After assembling of the sequence, oligonucleotide support was treated with aqueous ammonia (32%) with 0.1 M DTT (1,4-dithiothreitol) for 2 h at room temperature. 18 The ammonia solutions were concentrated to dryness and the product was desalted on NAP-10 (Sephadex G-25) columns eluted with water prior to use. Free oligo-FdU synthesis: Control pentamer oligo-FdU without HEG and thiol groups was prepared as before but using 3′-succinyl-FdU controlled pore glass as solid support. Finally, the oligonucleotide was deprotected with aqueous ammonia (32%) for 2 h at room temperature. (b) Physicochemical characterization of the Oligo-(FdU) 5-SH. HPLC analysis of pentamer FdU-HEG-SH (Conditions: X-bridge™ OST C18 (10×50 mm, 2.5 μm); 20 min linear gradient from 0% to 40%, flow rate 2 mL/min; solution A was 5% ACN in 0.1 M aqueous triethylammonium acetate (TEAA) and B 70% ACN in 0.1 M aqueous TEAA. (c) UV spectra of pentamer FdU-HEG-SH. The pentamer was quantified by absorption at 260 nm. (d) MS spectrum (MALDI-TOF) of pentamer FdU-HEG-SH (oligo-FdU). M calculated 1976.2 M found 1974.0. (e) MS spectrum (MALDI-TOF) of control pentamer FdU (free oligo-FdU). M calculated 1478.1, M found 1476.5.

FIG. 2. Physicochemical characterization of the T22-GFP-H6-FdU nanoconjugate and drug to nanoparticle ratio. The analysis of the products of the conjugation was performed by MALDI-TOFF spectra. (a) Mass spectrometry of the T22-GFP-H6-FdU conjugation products identifying the molecular mass of the products carrying 1 oligo-FdU or 2 oligo-FdU payloads, the unconjugated T22-GFP-H6 protein and the coupler-conjugated T22-GFP-H6. (b) T22-GFP-H6-FdU size as determined by dynamic light scattering, as compared to T22-GFP-H6 nanoparticle. (c) Representative T22-GFP-H6-FdU image as detected by transmission electron microscopy. (d) Molecular modeling of T22-GFP-H6-FdU self-assembled nanoparticle (source: Rueda, F. et al. Adv Mater 27, 7816-7822 (2015). Printed with permission from John Wiley & Sons).

FIG. 3. Mouse models, experimental setting and T22-GFP-H6-FdU dosage used to evaluate antimetastatic effect in the regression and prevention of metastasis protocols. (a) Mouse model used in the regression of established metastasis protocol: Direct orthotopic implantation of CXCR4⁺ bioluminescent SW1417 CRC cells in Swiss nude mice, which generates metastases in the lymph nodes and lung. Scale bar, 1 cm. (b) T22-GFP-H6-FdU administration, at a dosage of 20 μg q3d per 10 doses, was started 2 months after cecal implantation of CRC cells (when established metastases are already present) to evaluate the nanoconjugate capacity to induce the regression of established metastases. (c) Mouse model used in the prevention of metastasis protocol: subcutaneous passage of CXCR4⁺ bioluminescent SW1417 cells followed by orthotopic implantation of disaggregated tumor cells (SC+ORT) in NOD/SCID mice, which generates metastases in LN, liver, lung and peritoneum. (d) T22-GFP-H6-FdU administration, at a dosage of 20 μg q3d per 12 doses, was started one week after cecal implantation of CRC cells (when no metastases could yet develop), to evaluate the nanoconjugate capacity to prevent metastasis development. In both experiments, T22-GFP-H6-FdU antimetastatic effect is compared to that achieved by equimolecular doses of free oligo-FdU. Prior to initiating the prevention of metastasis experiment we confirmed in separate mice that the mouse model used lacked micro or macrometastases before treatment start. Prior to the initiation of the regression of metastasis experiment, we confirmed in separate mice that metastatic foci were already present before treatment start. Scale bar, 100 μm.

FIG. 4. T22-GFP-H6-FdU nanoconjugate synthesis and demonstration of selective internalization and killing of CXCR4⁺ CRC cells in vitro. (a) The nanoconjugate contains a fusion protein, T22-GFP-H6 (described in Unzueta 2012)-composed of the peptide T22 as a CXCR4 ligand, a green fluorescent protein and a Histidine tail-bound to the payload drug. (b) Seven to nine pentameric oligonucleotides (approximately 40 molecules) of the antitumor drug 5-Fluoro-2′-deoxyUridine (FdU), named oligo-FdU, are conjugated to the T22-GFP-H6 targeting vector using a coupler. (c) T22-GFP-H6-FdU chemical synthesis: T22-GFP-H16 is first covalently bound to the MBHS (6-Maleimidohexanoic acid N-hydroxysuccinimide ester) coupler through its amino groups in the external lysines. The thiol-functionalized oligo-FdU (oligo-(FdU) 5-SH) is then reacted with T22-GFP-H6 functionalized with maleimide (Michael reaction). (d) Nanoconjugate internalization in CXCR4 overexpressing (CXCR4⁺) SW1417 CRC cells after 1 hour exposure at 1 μM, as measured by fluorescence emission using flow cytometry.

FIG. 5. T22-GFP-H6-FdU selectively internalizes and kills CXCR4⁺ HeLa cells in vitro. (a) T22-GFP-H6-FdU nanoconjugate or T22-GFP-H6 internalization in Hela cells detected as emitted fluorescence in by flow cytometry after 1 hour cell exposure at 1 μM concentration. Complete internalization block obtained by pre-treatment of cells with the CXCR4 antagonist ADMD3100. (b) Intracellular trafficking of T22-GFP-H6-FdU in HeLa cells by confocal microscopy after exposure at 1 μM for 24 h. (c) Linearized T22-GFP-H6-FdU dose-response trend line representation compared with free oligo-FdU exposure. Antitumor effect was measured as HeLa cell viability by MTT after 72 h exposure as the described concentrations. (d) Reduction of cell viability as recorded by optical microscope images of HeLa cells exposed to T22-GFP-H6-FdU nanconjugates for 48 h, as compared to T22-GFP-H6 or free oligo-FdU. Scale bar, 100 μm.

FIG. 6. Selective and receptor-dependent uptake of T22-GFP-H6-FdU in CXCR4⁺ cells in vivo. (a) Approach to achieve targeted drug delivery and selective killing of metastatic stem cells: CXCR4-nanoconjugate interaction triggers CXCR4-mediated internalization in MetSCs, in primary tumors and metastatic foci, followed by FdU release to the cytosol and diffusion to the nucleus to induce double stand breaks leading to selective killing of CXCR4⁺ cells. (b) Selective T22-GFP-H6-FdU nanoconjugate biodistribution in subcutaneous CXCR4⁺ SW1417 CRC tumor tissue 5 h after a 100 μg single intravenous dose, as measured by fluorescence emission using IVIS Spectrum 200. Biodistribution is similar to that achieved by the T22-GFP-H6 targeting vector and undetectable after buffer or free oligo-FdU treatment. (c) Detection of T22-GFP-H6-FdU accumulation in tumor tissue, at a level similar to T22-GFP-H6, as measured by anti-GFP IHC. Absence of GFP detection in Buffer or free oligo-FdU controls. (d) Administration of the CXCR4 antagonist AMD3100 completely blocks T22-GFP-H6-FdU tumor biodistribution, as measured by fluorescence emission. Fluorescence is not detected in Buffer or free oligo-FdU controls. (e) The uptake of T22-GFP-H6-FdU observed in CXCR4⁺ SW1417 tumor tissues is almost completely blocked by prior AMD3100 administration, as quantified using the anti-GFP IHC H-score. (f) Representative images of T22-GFP-H6-FdU uptake and AMD3100 competition by anti-GFP inmunostaining, which quantitation is reported in panel e. Scale bar, 50 μm.

FIG. 7. T22-GFP-H6-FdU-induced depletion of CXCR4-overexpressing cancer cells in tumor tissue and definition of dosage interval. (a) Similar level of CXCR4 overexpression in subcutaneous tumor tissue among compared groups (Buffer, T22-GFP-H6-FdU, T22-GFP-H6 and free oligo-FdU) before treatment (upper panels). Representative images of DNA double-strand break induction (measured with anti-gH2AX IHC, 5 h post-administration, middle panels) and apoptotic induction (Hoechst staining, at 24 h, lower panels). Note the higher level of DSBs and apoptosis induction in the T22-GFP-H6-FdU as compared to free oligo-FdU. White arrows indicate apoptotic cells. (b) T22-GFP-H6-FdU depletes CXCR4 cancer cells from SW1417 CRC tumor tissue after a 100 μg single dose administration. Note the reduction in CXCR4⁺ cell fraction in the tumor 24 h after injection, their complete elimination at 48 h and the re-emergence of CXCR4⁺ cells 72 h post-administration, to reach a percent similar to the basal level, using anti-CXCR4 IHC. In contrast, the CXCR4⁺ cancer cell fraction (CXCR4⁺ CCF) in tumor tissue remains constant along time after free oligo-FdU treatment. The three day time-lapse for CXCR4′ tumor cell re-appearance defines the dosage interval used in a repeated dose schedule of nanoconjugate administration in the experiments that evaluate its antimetastatic effect. Scale bar, 50 μm.

FIG. 8. T22-GFP-H6-FdU prevents metastasis in a CXCR4-dependent manner. (a) T22-GFP-H6-FdU blocks the appearance of bioluminescent lymph node (LN), liver and lung metastases (mets) in the CXCR4⁺ SW1417 orthotopic model at the end of the prevention of metastases experiment as compared to free oligo-FdU. (b) T22-GFP-H6-FdU prevents metastases by potently reducing the total number of liver, lung and peritoneal mets, as recorded in H&E stained histology sections at the end of the prevention of metastasis experiment, in comparison to free oligo-FdU. In contrast, the number of LN mets is not reduced after T22-GFP-H6-FdU or free oligo-FdU administration *P<0.05, Mann-Withney test. See Table 1 for the results on nanoconjugate-induced reduction in mean foci number. (c) T22-GFP-H6-FdU induces a higher reduction of the remaining CXCR4⁺ cancer cell fraction (CXCR4⁺ CCF) in liver, lung and peritoneal metastatic tissue at the end of the experiment than free oligo-FdU, as measured by anti-CXCR4 IHC. In contrast, T22-GFP-H6 FdU or free oligo-FdU do not reduce the CXCR4⁺ CCF remaining in LN mets or primary tumor tissue after therapy. (d) Representative images of the reduction in CXCR4⁺ CCF induced by T22-GFP-H6-FdU or free oligo-FdU at the end of therapy, which quantitation is reported in panel c. Note the correlation between the reduction in CXCR4⁺ CCF induced by T22-GFP-H6-FdU (panel c) and its antimetastatic effect at each metastatic site (panel b). Scale bar, 100 μm. Asterisks: tumor tissue; vL: lymphatic vessel, LN: lymphatic metastasis.

FIG. 9. T22-GFP-H6-FdU induces the regression of established metastases in a CXCR4-dependent manner. (a) T22-GFP-H6-FdU shows a higher inhibition of lung mets than free oligo-FdU, as measured by bioluminescence emission at the end of the regression of metastases experiment, whereas both compounds show a similar level of inhibition of LN mets in the CXCR4+ SW1417 orthotopic model. (b) T22-GFP-H6-FdU shows a higher reduction in the number of Lung mets than free oligo-FdU as recorded in H&E stained tissue sections at the end of the regression of metastasis experiment, whereas both compounds show a similar level of inhibition of LN mets.

FIG. 10. T22-GFP-H6-FdU inhibition of primary tumor growth. (a) In 78 the prevention of metastasis experiment, no significant inhibition in primary tumor growth was observed along the experimental time, as measured by in vivo bioluminescence emission, after T22-GFP-H6-FdU or free oligo-FdU treatment, as compared to buffer-treated mice. (b) Ex vivo recording of bioluminescene emitted by the primary tumor after its resection at the end of the experiment showed also similar levels among T22-GFP-H6-FdU, free oligo-FdU and Buffer-treated mice. (c) In the regression of established metastasis experiment, T22-GFP-H6-FdU inhibited primary tumor growth along the experimental time, as detected by in vivo bioluminescence emission (BL1), to levels similar to these achieved by free oligo-FdU. Both compounds significantly inhibited tumor growth as compared to buffer treated animals. (d) Ex vivo recording of bioluminescence emitted by the primary tumor, after its resection at the end of the experiment, showed also similar levels between the T22-GFP-H6-FdU and free oligo-FdU mice. Note that in contrast to registering a similar response of the primary tumor to T22-GFP-H6-FdU or free-oligo-FdU treatment, the anitmetastatic effect induced by T22-GFP-H6-FdU was significantly higher than that observed after free-oligo-FdU treatment, especially regarding metastasis prevention (see FIG. 4 and Table 1).

FIG. 11. Negligible T22-GFP-H6-FdU biodistribution or toxicity on non-tumor tissues. (a) Undetectable T22-GFP-H6-FdU emitted fluorescence in normal tissues, except for a transient accumulation 5 h after a 100 μg dose, in the liver, which disappears at 24 h. Liver emitted fluorescence is transient and significantly lower than the one registered in tumor tissue. Tumor/Liver ratio=7.5 (see tumor intensity in FIG. 2b, which was registered in the same experiment) Scale bar, 1 cm. (b) Representative images depicting a similarly low level of DNA double strand break induction in normal bone marrow 5 h after T22-GFP-H6-FdU or free oligo-FdU administration, as measured by anti-g-H2AX, an effect that is not present in any other normal tissue analyzed. (c) Representative images depicting the lack of histopathological alterations in H&E stained tissue or apoptotic induction in H&E stained samples of CXCR4⁺ (bone marrow) and CXCR4− (brain, kidney, liver, lung and heart) normal tissues 24 h after the administration of a 100 μg dose of T22-GFP-H6-FdU or an equimolecular 554 dose of free oligo-FdU. Note that the transient nanoconjugate distribution to liver or the DNA damage induced in bone marrow do not lead to cytoxicity on these no-tumor tissues. (d) Lack of differences in body weight among groups registered along time in the regression of metastases experiment. (e) Lack of differences in body weight among groups registered along time in the prevention of metastases experiment. Scale bar, 100 μm.

FIG. 12. Undetectable T22-GFP-H6-FdU accumulation in bone marrow 92 or circulating blood cells. (a) Lack of fluorescence emission in erythrocytes, leucocytes or platelets isolated from blood using a Ficoll density gradient 5 h after the administration of T22-GFP-H6-FdU at doses in the 10-100 μg range, or control equimolecular doses of free FdU (1.3-13.0 nmol range). (b) Undetectable fluorescence emission observed in isolated leukocytes and platelet pellets after the Ficoll protocol. (c) Lack of fluorescence emission in spleen or bone marrow obtained 5 h after the treatment of mice with single injections of T22-GFP-H6-FdU or control free oligo-FdU in the dose range described in (a).

FIG. 13. Physicochemical characterization of the T22-GFP-H6-FdU nanoconjugate and demonstration of selective internalization and killing of CXCR4⁺ CRC cells in vitro. (A). Mass spectrometry of the initial T22-GFP-H6 protein and the T22-GFP-H6-FdU conjugates. (B) DLS of the T22-GFP-H6-FdU conjugate using inverted reaction. (C) Dose-response representation of CXCR4+ HeLa cells exposed to different concentration of T22-GFP-H6-FdU conjugates for 48 h, analyzed by MTT viability assay.

### DETAILED DESCRIPTION OF THE INVENTION

The inventors tested the principle behind the architecture of nanostructured proteins through the use of fusion proteins which included polycationic peptides with cell selectivity effects and proteins without an inherent physiological or biological activity. The fusion protein, later conjugated with therapeutic agents, the inventors observed that, surprisingly, the fusion proteins functioned as an effective target-selective delivery system for the therapeutic agents.

### Fusion Proteins of the Invention

Thus, in a first aspect, the invention relates to a fusion protein comprising
(i) a polycationic peptide,
(ii) an intervening polypeptide region and
(iii) a positively charged amino acid-rich region,
wherein the intervening polypeptide region is conjugated to at least one therapeutic agent.

The term “fusion protein” is well known in the art, referring to a single polypeptide chain artificially designed which comprises two or more sequences from different origins, natural and/or artificial. The fusion protein, per definition, is never found in nature as such.

The term “single polypeptide chain”, as used herein means that the polypeptide components of the fusion protein can be conjugated end-to-end but also may include one or more optional peptide or polypeptide “linkers” or “spacers” intercalated between them, linked by a covalent bond.

The term “peptide” or “polypeptide”, as used herein, generally refers to a linear chain of around 2 to 40 amino acid residues joined together with peptide bonds. It will be understood that the terms “peptide bond”, “peptide”, “polypeptide” and protein are known to the person skilled in the art. From here on, “peptide” and “polypeptide” will be used indistinctly.

As used herein, an “amino acid residue” refers to any naturally occurring amino acid, any amino acid derivative or any amino acid mimic known in the art. In certain embodiments, the residues of the protein or peptide are sequential, without any non-amino acid interrupting the sequence of amino acid residues. In other embodiments, the sequence may comprise one or more non-amino acid moieties. In particular embodiments, the sequence of residues of the protein or peptide may be interrupted by one or more non-amino acid moieties.

The term “conjugate”, as used herein, refers to any compound resulting from the covalent attachment of two or more individual compounds. In the present invention, conjugate refers to the intervening polypeptide region and at least one therapeutic agent which are covalently coupled, being said coupling direct or via a linking compound.

The terms “covalent coupling” or “covalent attachment” mean that the polypeptide region and at least one therapeutic agent are either directly covalently joined through a chemical covalent bond to one another, or else are indirectly covalently joined to one another through an intervening moiety or moieties, such as a linker, or a bridge, or a spacer, moiety or moieties.

### A. The Polycationic Peptide

The term “polycationic peptide” or “first positively charged amino acid-rich region” as used herein, corresponds to a polypeptide sequence containing multiple positively charged amino acids. The polycationic peptide may be formed exclusively by positively charged amino acids or may contain other amino acids provided that the overall net charge of the region at pH 7 is positive.

It is well known in the art that amino acids and their corresponding amino acid residues possess different properties depending on their side chains and they may be grouped depending on those properties. Thus, at physiological pH, five amino acids show an electrical charge: arginine, histidine, and lysine are positively charged while aspartic acid and glutamic acid are negatively charged. The person skilled in the art will acknowledge then that the polycationic peptide of the invention corresponds to a polypeptide with a net electrical charge of more than one positive charge in physiological pH conditions. Accordingly, the polycationic peptide of the invention is not limited by the presence of one or more negatively charge amino acid residues as long as there are always enough positively charged amino acid residues to result in a net positive electrical charge of two or more.

Thus, in one embodiment of the invention, the polycationic peptide of the invention is selected from the group consisting of
(i) an arginine-rich sequence,
(ii) a sequence which is capable of specifically interacting with a receptor on a cell surface and promoting internalization of the fusion protein on said cell,
(iii) the GW-H1 peptide,
(iv) a CD44 ligand,
(v) a peptide capable of crossing the blood-brain barrier,
(vi) a cell penetrating peptide and
(vii) a nucleolin-binding peptide.

### (i) Arginine-Rich Sequence

As aforementioned, the arginine amino acid and its residue present positive charge at physiological pH. It will be understood that an “arginine-rich sequence” refers to a polypeptide sequence containing multiple arginine residues. Thus, the polypeptide sequence may comprise 33%, preferably 40%, preferably 45%, preferably 50%, preferably 55%, preferably 60%, preferably 65%, preferably 70%, preferably 75%, preferably 80%, preferably 85%, more preferably 90%, more preferably 95%, even more preferably 99%, yet even more preferably 100% of the amino acid residues of its complete sequence as arginine residues. It will be understood that whenever the sequence of the arginine-rich sequence comprises less than the 100% of the sequence as arginine residues, these residues do not need to be all adjacent or contiguous with respect to each other.

The person skilled in the art will recognize that a polypeptide with one or more arginine residues will be a polycationic peptide as long as the total positive electrical charge of the polypeptide at physiological pH is 2 or more, resulting not only from the positive electrical charges of the arginine residues but also from any other positively charged amino acids.

In an embodiment of the invention, the polycationic peptide of the invention is an arginine-rich sequence.

In a preferred embodiment of the invention, the arginine-rich sequence of the polycationic peptide of the invention is selected from the group consisting of SEQ ID NO: 1, SEQ ID NO: 2, SEQ ID NO: 3 and SEQ ID NO: 4.

(ii) Sequence which is Capable of Specifically Interacting with a Receptor on a Cell Surface and Promoting Internalization of the Fusion Protein on Said Cell

The terms “sequence which is capable of specifically interacting with a receptor on a cell surface and promoting internalization of the fusion protein on said cell”, as used herein, refers to any polypeptide sequence which binds to a receptor on the surface of a cell, wherein the receptor undergoes endocytosis in response to the binding of said polypeptide sequence. This binding specificity allows the delivery of the polypeptide sequence, as well as the rest of the fusion protein which it is a part of, to the cell, tissue or organ which expresses said receptor. In this way, a fusion protein comprising said polypeptide sequence will be directed specifically to said cells when administered to an animal or contacted in vitro with a population of cells of different types.

The term “receptor” denotes a cell-associated protein that binds to a bioactive molecule termed a “ligand”. Both “receptor” and “ligand” are commonly known to those skilled in the art.

As used herein, “internalization” refers to a process by which a molecule or a construct comprising a molecule binds to a target element on the outer surface of the cell membrane and the resulting complex is internalized by the cell. Internalization may be followed up by dissociation of the resulting complex within the cytoplasm. The target element, along with the molecule or the construct, may then localize to a specific cellular compartment. Preferably, the polycationic peptide of the invention, besides promoting internalization, will facilitate endosomal escape of the fusion protein.

A wide array of uptake receptors and carriers, with an even wider number of receptor-specific ligands, are known in the art.

Non-limiting examples of receptors which may be targeted by the polycationic of the invention include an angiotensin receptor, a bombesin receptor, a bradykinin receptor, a calcitonin receptor, a chemokine receptor, a cholecystokinin receptor, a corticotropin-releasing factor receptor, an endothelin receptor, an ephrin receptor, a formylpeptide receptor, a Frizzled receptor, a galanin receptor, a the growth hormone secretagogue receptor (Ghrelin) receptor, a Kisspeptin receptor, a melanocortin receptor, Neuropeptide FF/neuropeptide AF receptor, a neuropeptide S receptor, a neuropeptide W/neuropeptide B receptor, a neuropeptide Y receptor, a neurotensin receptor, an orexin receptors, a peptide P518 receptor, a somatostatin receptor, a tachykinin receptor, a Toll-like receptor, a vasopressin and oxytocin receptor and a VEGF receptor.

In a preferred embodiment of the invention, the polycationic peptide comprising a sequence which is capable of specifically interacting with a receptor on a cell surface and promoting internalization of the fusion protein on said cell is a CXCR4 ligand.

The term “CXCR4”, as used herein, refers to a G protein-coupled, seven-transmembrane chemokine receptor. Like other chemokine receptors, CXCR4 plays an important role in immune and inflammatory responses by mediating the directional migration and activation of leukocytes CXCR4 is expressed or overexpressed in a variety of cancer cell lines and tissues including breast, prostate, lung, ovarian, colon, pancreatic, kidney, and brain, as well as non-Hodgkin's lymphoma and chronic lymphocytic leukemia. The only known ligand to CXCR4 is stromal cell-derived factor-1 (SDF-1, or CXCL12). The interaction between CXCR4 and SDF-1 plays an important role in multiple phases of tumorigenesis, including tumor growth, invasion, angiogenesis, and metastasis.

The expression “specifically binding to CXCR4”, as used herein refers to the ability of the conjugates of the invention to bind more frequently, more rapidly, with greater duration and/or with greater affinity to CXCR4 or cell expressing same than it does with alternative receptors or cells without substantially binding to other molecules.

Binding affinity is measured, for instance, as described by Tamamura et al. by the oil-cushion method [see Hesselgesset et al, 1998, J. Immunol., 160:877-883] comprising contacting the peptide with CXCR4-transfected cell line (e.g. CHO cells) and a labeled CXCR4 ligand (e.g. ¹²⁵I-SDF-1α) and measuring the inhibition percentage of the targeting peptide against the binding of the labeled CXCR4 ligand.

Specific binding can be exhibited, e.g., by a low affinity targeting agent having a Kd of at least about 10⁻⁴ M. e.g., if CXCR4 has more than one binding site for a ligand, a ligand having low affinity can be useful for targeting. Specific binding also can be exhibited by a high affinity ligands, e.g. a ligand having a Kd of at least about of 10⁻⁷ M, at least about 10⁻⁸ M, at least about 10⁻⁹ M, at least about 10⁻¹⁰ M, or can have a Kd of at least about 10⁻¹¹ M or 10⁻¹² M or greater. Both low and high affinity-targeting ligands are useful for incorporation in the conjugates of the present invention.

The ability of the conjugate of the invention to be internalized by cells expressing CXCR4 may be conveniently determined by fluorescence methods in the case that the conjugate comprises a fluorescent protein, such as GFP. Such fusion proteins can be obtained by preparing a recombinant nucleic acid wherein the nucleic acids encoding the T22 peptide and the fluorescent protein are fused in frame and expressed in an adequate host cell or organism. The fusion protein is then contacted with a culture of cells expressing CXCR4 or in vivo with a tissue which expresses CXCR4 for an appropriate amount of time, after which fluorescence microscopy may be used to determine whether the construct penetrated the cell. Presence of fluorescence in the cytoplasm may be further investigated by comparing the fluorescence microscopy image resulting from the fluorescent protein to that obtained with a known cytoplasmic stain.

The expression “facilitate endosomal escape”, as used herein, refers to the ability of the polycationic peptide to induce the release of the fusion proteins from the endosomal compartment after internalization by receptor-mediated endocytosis.

In an even more preferred embodiment of the invention, the CXCR4 ligand is selected from the group comprising the T22 peptide (SEQ ID NO: 5), the V1 peptide (SEQ ID NO: 6), the CXCL12 peptide (SEQ ID NO: 7), the vCCL2 peptide (SEQ ID NO: 8) or a functionally equivalent variant thereof.

The T22 peptide corresponds to a peptide derived from the protein polyphemusin II (extracted from hemocyte debris from Lymulus polyphemus). The vCCL2 corresponds to the viral macrophage inflammatory protein-II, an homologue of human chemokine CCL2 encoded by human herpesvirus 8. The V1 peptide corresponds to residues 1-21 of the N-terminus of vCCL2. CXCL12, C-X-C motif chemokine 12, also known as stromal cell-derived factor 1 (SDF1), is a member of the chemokine family that acts as a pro-inflammatory mediator. All four peptides are known to have interactions with the CXCR4 receptor, as shown in Liang, X. 2008. Chem. Biol. Drug. Des. 72:91-110.

In one embodiment, the targeting peptide is the selected from the group consisting of:
the T140 peptide having the sequence RRX₁CYRKX₂PYRX₃CR (SEQ ID NO: 9) wherein X₁ is L-3-(2-naphtyl) alanine, X₂ is D-Lys and X₃ is L-Citrulline.
the TN14003 peptide having the sequence RRX₁CYX₂KX₃PYRX₄CR (SEQ ID NO: 10) wherein X₁ is L-3-(2-naphtyl) alanine, X₂ is L-Citrulline, X₃ is dLys and X₄ is L-Citrulline,
the TC14012 peptide having the sequence RRX₁CYEKX₂PYRX₃CR (SEQ ID NO: 11) wherein X₁ is L-3-(2-naphtyl) alanine, X₂ is D-Citrulline and X₃ is L-Citrulline,
the TE14011 peptide having the sequence RRX₁CYX₂KX₃PYRX₄CR (SEQ ID NO: 12) wherein X₁ is L-3-(2-naphtyl) alanine, X₂ is L-Citrulline, X₃ is D-Glu and X₄ is L-Citrulline and
the TZ14011 peptide having the sequence RRX₁CYX₂KX₃PYRX₄CR (SEQ ID NO: 13) wherein X₁ is L-3-(2-naphtyl) alanine, X₂ is L-Citrulline, X₃ is D-Lys and X₄ is L-Citrulline or the variant thereof wherein the N-terminal Arginine residue is acetylated (known Ac-TZ14011).

The terms “functional variant” and “functionally equivalent variant” are interchangeable and are herein understood as all those peptides derived from the T22, the V1, the CXCL12, and/or the vCCL2 peptides by means of modification, insertion and/or deletion of one or more amino acids, provided that the function of binding to CXCR4 and internalizing the fusion protein is substantially maintained.

In one embodiment, functionally equivalent variants of the cationic polypeptides are those showing a degree of identity with respect to the human T22, V1, CXCL12 and/or the vCCL2 peptides, according to their respective SEQ ID NOs, greater than at least 40%, at least 50%, at least 60%, at least 70%, at least 80%, at least 85%, at least 90%, at least 95%, at least 96%, at least 97%, at least 98% or at least 99%. The degree of identity between two amino acid sequences can be determined by conventional methods, for example, by means of standard sequence alignment algorithms known in the state of the art, such as, for example BLAST [Altschul S. F. et al., J. Mol. Biol., 1990 Oct. 5; 215 (3): 403-10]. The cationic polypeptides of the invention may include post-translational modifications, such as glycosylation, acetylation, isoprenylation, myristoylation, proteolytic processing, etc.

Alternatively, suitable functional variants of the cationic polypeptide are those wherein one or more positions contain an amino acid which is a conservative substitution of the amino acid present in the T22, V1, CXCL12, and/or vCCL2 peptides mentioned above. “Conservative amino acid substitutions” result from replacing one amino acid with another having similar structural and/or chemical properties For example, the following six groups each contain amino acids that are conservative substitutions for one another: 1) Alanine (A), Serine(S), Threonine (T); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); and 6) Phenylalanine (F), Tyrosine (Y), Tryptophan (W). Selection of such conservative amino acid substitutions is within the skill of one of ordinary skill in the art and is described, for example by Dordo et al. et al., [J. Mol. Biol, 1999, 217; 721-739] and Taylor et al., [J. Theor. Biol., 1986, 119:205-218].

A suitable assay for determining whether a given peptide can be seen as a functionally equivalent variant thereof is, for instance, the following assay: a putative T22, V1, CXCL12 or vCCL2 peptide variant is fused in frame with a marker polypeptide (e.g. a fluorescent protein). Such fusion proteins can be obtained by preparing a recombinant nucleic acid wherein the nucleic acids encoding the peptide and the fluorescent protein are fused in frame and expressed in an adequate host cell or organism. The fusion protein is then contacted with a culture of cells CXCR4 (e.g. HeLa cells) for an appropriate amount of time after which fluorescence microscopy may be used to determine whether the construct penetrated the cell. If the peptide is a functionally equivalent variant of the corresponding peptide, the marker protein will be internalized and presence of fluorescence in the cytoplasm of the cell will be visible. Furthermore, the performance of the functionally equivalent variant can be assayed by comparing the fluorescence microscopy image resulting from the fluorescent protein to that obtained with a known cytoplasmic stain (e.g. DAPI).

(iii) The GW-H1 Peptide

The GW-H1 peptide was previously described by Chen and colleagues [Chen, Y-L. S. et al. 2012. Peptides, 36:257-265]. The GW-H1 peptide was first selected as an antimicrobial peptide but it is also characterized by its capability to bind to cell membranes, internalize itself to the cytoplasm, and migrate to the nuclei in eukaryotic cells. Once inside the cell, GW-H1 is capable induce apoptosis. It has been proposed that GW-H1 exerts its cytolytic activity by folding into an amphipathic helix [Chen and colleagues, supra]. Therefore, this peptide is supposed to exert its cell lytic effects by two sequential events consisting on binding to cell membranes followed by permeabilization.

In a preferred embodiment of the invention, the polycationic peptide of the invention is the GW-H1 peptide, which has the SEQ ID NO: 14.

### (iv) A CD44 Ligand

CD44 is a cell-surface transmembrane glycoprotein involved in cell-cell and cell-matrix interactions, cell adhesion and migration. CD44 has been implicated in inflammation and in diseases such as cancer [Bajorath, J. 2000. Proteins. 39:103-111]. Many isoforms are known, which are expressed in a cell-specific manner and also differentially glycosylated

Accordingly, a “CD44 ligand” will be a molecule capable of binding to CD44. CD44 is the major surface receptor for Hyaluronan, a component of the extracellular matrix, but it has other ligands, such as chondroitin sulfate, the heparin-biding domain of fibronectin, osteopontin, serglycin, collagen and laminin. Besides, CD44 can also interact with metalloproteinases and selectins.

In an embodiment of the invention, the polycationic peptide of the invention is a CD44 ligand. In a preferred embodiment of the invention, the CD44 ligand is selected from the group consisting of A5G27 (SEQ ID NO: 15) and FNI/II/V (SEQ ID NO: 16).

The peptide FNI/II/V corresponds to the HBFN-fragment V of Fibronectin. The peptide A5G27 corresponds to a peptide of the α5 chain of Laminin [Pesarrodona, M. et al. 2014. Int. J. of Pharmaceutics. 473:286-295].

### (v) Peptide Capable of Crossing the Blood-Brain Barrier

It is well known in the art that one major obstacle for the development of therapeutic approaches for brain pathologies is the blood-brain barrier (BBB). The brain is shielded against potentially toxic substances by the presence of two barrier systems: the blood-brain barrier (BBB) and the blood-cerebrospinal fluid barrier (BCSFB). The BBB is considered to be the major route for the uptake of serum ligands since its surface area is approximately 5000-fold greater than that of BCSFB. The brain endothelium, which constitutes the BBB, represents the major obstacle for the use of potential drugs against many disorders of the CNS. As a general rule, only small lipophilic molecules may pass across the BBB, i.e., from circulating systemic blood to brain. Many drugs that have a larger size or higher hydrophobicity show promising results in animal studies for treating CNS disorders.

Therefore, a “peptide capable of crossing the blood-brain barrier” will be a peptide capable of transporting itself as well as any molecule it is bound to, preferably a protein, from the blood torrent to the CNS.

In 1983 it was reported that a peptide, β-Casomorphin-5 could overcome the BBB [Ermisch, A. et al. 1983. J. of Neurochemistry. 41:1229-1233]. Since then, many other peptides with BBB-permeating properties have been identified, characterized and catalogued, and in 2012 a comprehensive database was established, as reported by Van Dorpe et al. [Van Dorpe, S. et al. 2012. Brain Struct. Funct. 217:687-718]. Most of the peptides listed in the aforementioned database are suitable for the fusion protein of the invention.

In an embodiment of the invention, the polycationic peptide of the invention is a peptide capable of crossing the blood-brain barrier. In a preferred embodiment of the invention, the peptide capable of crossing the blood-brain barrier is a selected from the group consisting of Seq-1-7 (SEQ ID NO: 17), Seq-1-8 (SEQ ID NO: 18), and Angiopep-2-7 (SEQ ID NO: 19).

### (vi) Cell Penetrating Peptide (CPP)

The terms “cell-penetrating peptide” (CPP) refers to a peptide, typically of about 5-60 amino acid residues in length, that can facilitate cellular uptake of molecular cargo, particularly proteins they are a part of. Proteins can present one or more CPPs. CPPs can also be characterized as being able to facilitate the movement or traversal of molecular cargo across/through one or more of a lipid bilayer, cell membrane, organelle membrane, vesicle membrane, or cell wall. A CPP herein will be polycationic.

Examples of CPPs useful herein, and further description of CPPs in general, are disclosed in Schmidt et al. [2010. FEBS Lett. 584:1806-1813], Holm et al. [2006. Nature Protocols 1:1001-1005], Yandek et al, [2007. Biophys. J. 92:2434-2444], Morris et al. [2001. Nat. Biotechnol. 19:1173-1176]. and U.S. Patent Application Publication No. 2014/0068797. CPPs do not depend on transporters or receptors, facilitating the traffic of the proteins they are part of directly through the lipid bilayer without the need of participation by any other cell components.

(vii) Nucleolin-Binding Peptide

Accordingly, a “nucleolin-binding peptide” is a peptide capable of binding to the nucleolin protein in a cell, preferably to the cell-surface expressed fraction of nucleolin. In an embodiment of the invention, the polycationic peptide of the invention is a nucleolin-binding peptide.

The International Patent Application Publication with number WO 2011/031477 A2 offers numerous examples of nucleolin-binding peptides that would be suitable for use in the fusion protein of the invention.

In a preferred embodiment of the invention, the nucleolin-binding peptide of the invention is the peptide of sequence SEQ ID NO: 20 or the peptide of sequence SEQ ID NO: 21.

### B. Positively Charged Amino Acid-Rich Region

The term “positively charged amino acid” or “second positively charged amino acid-rich region” as used herein, refers to a polypeptide sequence, different from the polycationic region or first positively charged amino acid-rich region characterized in that it contains multiple positively charged amino acids. In addition, the positively charged amino acid-rich region may be formed exclusively by positively charged amino acids or may contain other amino acids provided that the overall net charge of the region at pH 7 is positive. Thus, the positively charged amino acid-rich region sequence may comprise 33%, preferably 40%, preferably 45%, preferably 50%, preferably 55%, preferably 60%, preferably 65%, preferably 70%, preferably 75%, preferably 80%, preferably 85%, more preferably 90%, more preferably 95%, even more preferably 99%, yet even more preferably 100% of the amino acid residues of its complete sequence as positively charged amino acids residues.

The positively charged amino acid-rich region may contain only one type of positively charged amino acid or may contain more than one type of positively charged amino acid. In one embodiment, the positively charged amino acid-rich region is a polyhistidine region. In one embodiment, the positively charged amino acid-rich region is a polyarginine region. In one embodiment, the positively charged amino acid-rich region is a polyhistidine region. In one embodiment, the positively charged amino acid-rich region comprises lysine and arginine residues. In one embodiment, the positively charged amino acid-rich region comprises lysine and histidine residues. In one embodiment, the positively charged amino acid-rich region comprises arginine and histidine residues. In one embodiment, the positively charged amino acid-rich region comprises lysine, arginine and histidine residues

In some embodiments, the positively charged amino acid-rich region comprises at least 2, at least 3, at least 4, at least 5, at least 6, at least 7, at least 8, at least 9, at least 10, at least 11, at least 12, at least 13, at least 14, or at least 15 positively charged amino acids residues, wherein the positively charged amino acid can be histidine, lysine, arginine or combinations thereof.

In some embodiments, the positively charged amino acid-rich region comprises fewer than 100, fewer than 90, fewer than 80, fewer than 70, fewer than 60, fewer than 50, fewer than 40, fewer than 30, fewer than 29, fewer than 28, fewer than 27, fewer than 26, fewer than 25, fewer than 24, fewer than 23, fewer than 22, fewer than 21, fewer than 20, fewer than 19, fewer than 18, fewer than 17, fewer than 16, fewer than 15, fewer than 14, fewer than 13, fewer than 12, fewer than 11, fewer than 10 or less positively charged amino acids residues, wherein the positively charged amino acid can be histidine, lysine, arginine or combinations thereof.

In some embodiments, the positively charged amino acid-rich region comprises between 2 and 50 amino acids, between 2 and 40 amino acids, between 2 and 30 amino acids, between 2 and 25 amino acids, between 2 and 20 amino acids, between 2 and 10 amino acids or between 2 and 8 amino acids.

In some embodiments, the positively charged amino acid-rich region comprises between 3 and 50 amino acids, between 3 and 40 amino acids, between 3 and 30 amino acids, between 3 and 25 amino acids, between 3 and 20 amino acids, between 3 and 10 amino acids or between 3 and 8 amino acids. In some embodiments, the positively charged amino acid-rich region comprises between 4 and 50 amino acids, between 4 and 40 amino acids, between 4 and 30 amino acids, between 4 and 25 amino acids, between 4 and 20 amino acids, between 4 and 10 amino acids or between 4 and 8 amino acids. In some embodiments, the positively charged amino acid-rich region comprises between 5 and 50 amino acids, between 5 and 40 amino acids, between 5 and 30 amino acids, between 5 and 25 amino acids, between 5 and 20 amino acids, between 5 and 10 amino acids or between 5 and 8 amino acids.

In an embodiment of the invention, the positively charged amino acid-rich region of the fusion protein of the invention is a polyhistidine region. In a preferred embodiment of the invention, the polyhistidine region comprises between 2 and 10 contiguous histidine residues.

In an embodiment of the invention, the positively charged amino acid-rich region of the fusion protein of the invention is a polyarginine region. In a preferred embodiment of the invention, the polyarginine region comprises between 2 and 10 contiguous arginine residues.

In an embodiment of the invention, the positively charged amino acid-rich region of the fusion protein of the invention is a polylysine region. In a preferred embodiment of the invention, the polylysine region comprises between 2 and 10 contiguous polylysine residues.

### C. Relative Positions of the Elements of the Fusion Proteins and Linking Elements

The different elements of the fusion protein (polycationic peptide, intervening polypeptide region, and positively charged amino acid-rich region) of the invention can be placed in any relative order provided that the polycationic peptide and the positively charged amino acid-rich region are functional on any position of the fusion protein and also the intervening polypeptide region remains functional totally or in part.

As used herein, the terms “N-terminal end”, “N-terminus”, and “amino-terminal end” of a polypeptide are indistinct. Equally, the terms “C-terminal end”, “C-terminus”, and “carboxi-terminal end” are considered equivalent. The terms are of common usage for the person skilled in the art regarding the free moieties of the amino acids at the ends of the polypeptide chains comprised by a protein.

Thus, in an embodiment of the invention, the polycationic peptide of the fusion protein is located at the N-terminal end of the protein, while the positively charged amino acid-rich region of the fusion protein is located at the C-terminal end of the protein. In another embodiment of the invention, the positively charged amino acid-rich region of the fusion protein is located at the N-terminal end of the protein, while the polycationic peptide of the fusion protein is located at the C-terminal end of the protein. In another embodiment of the invention, the intervening polypeptide region can be located at either the C-terminal end or the N-terminal end of the fusion protein, while the polycationic peptide is in the middle position of the fusion protein and the positively charged amino acid-rich region is at the end of the fusion protein opposite the Intervening polypeptide region, or the positively charged amino acid-rich region is in the middle position of the fusion protein and the polycationic peptide is located at the end of the fusion protein opposite the Intervening polypeptide region.

Accordingly, the relative order of the elements of the fusion protein according to the invention, can be:
N-Polycationic peptide-Intervening region polypeptide-positively charged amino acid-rich region-C;
N-positively charged amino acid-rich region-Intervening region polypeptide-Polycationic peptide-C;
N-Polycationic peptide-positively charged amino acid-rich region-Intervening region polypeptide-C;
N-positively charged amino acid-rich region-Polycationic peptide-Intervening region polypeptide-C;
N-Intervening region polypeptide-Polycationic peptide-positively charged amino acid-rich region-C; or
N-Intervening region polypeptide-positively charged amino acid-rich region-Polycationic peptide-C

The terms “N-terminal end” and “C-terminal end” do not mean that the components need to be directly conjugated end-to-end, but that they maintain that relative order of positions regardless of the presence of additional elements at the end of either component or intercalated between them, such as linkers/spacers.

Therefore, the fusion protein of the invention comprises the aforementioned elements ((1) polycationic peptide, (2) intervening polypeptide region, and (3) positively charged amino acid-rich region) and these can be conjugated end-to-end but also may include one or more optional peptide or polypeptide “linkers” or “spacers” intercalated between them, linked, preferably by peptidic bond.

According to the invention, the spacer or linker amino acid sequences can act as a hinge region between components (1) and (2) and (2) and (3), allowing them to move independently from one another while maintaining the three-dimensional form of the individual domains, such that the presence of peptide spacers or linkers does not alter the functionality of any of the components (1), (2) and (3). In this sense, a preferred intermediate amino acid sequence according to the invention would be a hinge region characterized by a structural ductility allowing this movement. In a particular embodiment, said intermediate amino acid sequence is a flexible linker. The effect of the linker region is to provide space between the components (1) and (2) and (2) and (3). It is thus assured that the secondary and tertiary structure of component (1), (2) or (3) is not affected by the presence of either of the others. The spacer is of a polypeptide nature. The linker peptide preferably comprises at least 2 amino acids, at least 3 amino acids, at least 5 amino acids, at least 10 amino acids, at least 15 amino acids, at least 20 amino acids, at least 30 amino acids, at least 40 amino acids, at least 50 amino acids, at least 60 amino acids, at least 70 amino acids, at least 80 amino acids, at least 90 amino acids or approximately 100 amino acids.

The spacer or linker can be bound to components flanking the two components of the conjugates of the invention by means of covalent bonds, preferably by peptide bonds; and also preferably the spacer is essentially afunctional, and/or is not prone to proteolytic cleavage, and/or does not comprise any cysteine residue. Similarly, the three-dimensional structure of the spacer is preferably linear or substantially linear.

The preferred examples of spacer or linker peptides include those that have been used to bind proteins without substantially deteriorating the function of the bound peptides or at least without substantially deteriorating the function of one of the bound peptides. More preferably the spacers or linkers used to bind peptides comprise coiled coil structures.

Preferred examples of linker peptides comprise 2 or more amino acids selected from the group consisting of glycine, serine, alanine and threonine. A preferred example of a flexible linker is a polyglycine linker. The possible examples of linker/spacer sequences include SGGTSGSTSGTGST (SEQ ID NO: 22), AGSSTGSSTGPGSTT (SEQ ID NO: 23) or GGSGGAP (SEQ ID NO: 24). These sequences have been used for binding designed coiled coils to other protein domains [Muller, K. M., Arndt, K. M. and Alber, T., Meth. Enzymology, 2000, 328:261-281]. Further non-limiting examples of suitable linkers comprise the amino acid sequence GGGVEGGG (SEQ ID NO: 25), the sequence of 10 amino acid residues of the upper hinge region of murine IgG3 (PKPSTPPGSS, SEQ ID NO: 26), which has been used for the production of dimerized antibodies by means of a coiled coil [Pack, P. and Pluckthun, A., 1992, Biochemistry 31:1579-1584], the peptide of sequence APAETKAEPMT (SEQ ID NO: 27), the peptide of sequence GAP, the peptide of sequence AAA and the peptide of sequence AAALE (SEQ ID NO: 46).

Alternatively, the components of the conjugates of the invention can be connected by peptides the sequence of which contains a cleavage target for a protease, thus allowing the separation of any of the components. Protease cleavage sites suitable for their incorporation into the polypeptides of the invention include enterokinase (cleavage site DDDDK, SEQ ID NO: 28), factor Xa (cleavage site IEDGR, SEQ ID NO: 29), thrombin (cleavage site LVPRGS, SEQ ID NO: 30), TEV protease (cleavage site ENLYFQG, SEQ ID NO: 31), PreScission protease (cleavage site LEVLFQGP, SEQ ID NO: 32), inteins and the like.

Thus, in an embodiment of the invention, the polycationic peptide is bound to the intervening polypeptide region through a linker. In another embodiment of the invention, the intervening polypeptide region is bound to the positively charged amino acid-rich region through a linker. In yet another embodiment of the invention, the polycationic peptide is bound to the intervening polypeptide region through a linker and the intervening polypeptide region is bound to the positively charged amino acid-rich region through a linker also.

As the person skilled in the art will acknowledge, the linkers connecting the polycationic peptide to the intervening polypeptide region and the intervening polypeptide region to the positively charged amino acid-rich region may comprise the same sequence or different ones with the aforementioned limitation that the presence and/or sequence of the linkers does not result in functional alterations of the polycationic peptide, the intervening polypeptide region, and/or the positively charged amino acid-rich region (for instance, but not limited to, due to secondary or tertiary structure modifications of the fusion protein or formation of disulfide bonds).

The aforementioned considerations regarding the relative positions from the N-terminal end to the C-terminal end of the elements of the fusion protein apply also in the presence of linkers between them, independently of the number of them or what elements they are placed between. Therefore, the possible combinations and relative orders of elements will be the following (wherein the numbering stated above for the elements is retained: (1) polycationic peptide, (2) intervening polypeptide region, (3) positively charged amino acid-rich region):
N-(1)-(2)-(3)-C
N-(1)-linker-(2)-(3)-C
N-(1)-(2)-linker-(3)-C
N-(1)-linker-(2)-linker-(3)-C
N-(3)-(2)-(1)-C
N-(3)-linker-(2)-(1)-C
N-(3)-(2)-linker-(1)-C
N-(3)-linker-(2)-linker-(3)-C
N-(2)-(1)-(3)-C
N-(2)-linker-(1)-(3)-C
N-(2)-(1)-linker-(3)-C
N-(2)-linker-(1)-linker-(3)-C
N-(2)-(3)-(1)-C
N-(2)-linker-(3)-(1)-C
N-(2)-(3)-linker-(1)-C
N-(2)-linker-(3)-linker-(1)-C
N-(1)-(3)-(2)-C
N-(1)-(3)-linker-(2)-C
N-(1)-linker-(3)-(2)-C
N-(1)-linker-(3)-linker-(2)-C
N-(3)-(1)-(2)-C
N-(3)-linker-(1)-(2)-C
N-(3)-(1)-linker-(2)-C
N-(3)-linker-(1)-linker-(2)-C

In a preferred embodiment of the invention, the linkers of the fusion protein of the invention comprise the sequence GGSSRSS (SEQ ID NO: 33) sequence of the GGGNS sequence (SEQ ID NO: 34).

### D. Intervening Polypeptide Region

The terms “intervening polypeptide region” and “intervening region” are herein considered equivalent.

The intervening polypeptide region of the fusion proteins of the invention comprises a physiologically functional peptide, meaning that its interaction with the cellular components results in physiological changes. However, the intervening polypeptide region does not need to be physiologically functional once it is incorporated into the fusion protein of the invention. Accordingly, linker regions connecting the different elements of the fusion protein according to the invention are not considered intervening regions. Thus, in preferred embodiments, the intervening region comprises at least 5, at least 10, at least 15, at least 20, at least 25, at least 30, at least 35, at least 40, at least 50, at least 60, at least 70, at least 80, at least 90, at least 100 or more amino acids.

In a preferred embodiment the intervening polypeptide region is a physiologically functional peptide or a fragment or a mutant thereof with a reduced physiological function once it is incorporated into the fusion protein of the invention. In another embodiment, the intervening polypeptide region does not have any physiological function once incorporated into the fusion protein of the invention. In another preferred embodiment, the intervening polypeptide region is a fragment or a mutant of a physiologically functional polypeptide with an already reduced physiological function, as compared to the wild-type physiologically functional polypeptide before being incorporated into the fusion protein of the invention. More preferably, the intervening polypeptide region is a protein which does not have any physiological function already when not forming part of the fusion protein of the invention, due to the presence of inactivating mutations.

The intervening polypeptide region of the fusion proteins of the invention is a protein bound to a therapeutic agent.

The term “therapeutic”, as used herein in relation to the therapeutic agents, is used in a generic sense and includes treating agents, prophylactic agents, and replacement agents.

As it is part of the fusion protein of the invention with the polycationic peptide and the positively charged amino acid-rich region, the nature of the intervening region is substantially polypeptidic, except where the therapeutic agent is concerned. It is intended that the therapeutic agent conjugated to the fusion protein is not limited in its chemical structure.

In an embodiment of the invention, the intervening region of the fusion protein is selected from the group consisting of a fluorescent protein, albumin, nidogens, chorionic gonadotropin, and a cystatin.

“Fluorescent protein”, as used herein, relates to proteins whose atomic structure allows them to present fluorescence, which is a phenomenon well-known in the art. Non-limiting examples of commonly used fluorescent proteins suitable for the fusion protein of the invention, are the green fluorescent protein (GFP, first discovered in Aequorea victoria), the red fluorescent protein (RFP), the yellow fluorescent protein (YFP), the blue fluorescent protein (BFP), the cyan fluorescent protein, or any other variant, examples of which can be found in Kremers et al. [Kremers, G-J—et al. 2011. J. Cell Sci. 124:157-160].

Additional non-limiting examples of fluorescent proteins suitable for the fusion protein of the invention are the enhanced green fluorescent protein (eGFP), enhanced cyan fluorescent protein CFP (ECFP), enhanced YFP (EYFP), GFPS65T, Emerald, Topaz (TYFP), Venus, Citrine, mCitrine, GFPuv, destabilized EGFP (dEGFP), destabilised ECFP (dECFP), destabilized EYFP (dEYFP), mCFPm, Cerulean, T-Sapphire, CyPet, YPet, mKO, HcRed, t-HcRed, DsRed, DsRed2, DsRed-monomer, J-Red, dimer2, t-dimer2 (12), mRFPl, pocilloporin, Renilla GFP, Monster GFP, paGFP, Kaede protein and kindling protein, Phycobiliproteins and Phycobiliprotein conjugates including B-Phycoerythrin, R-Phycoerythrin and Allophycocyanin. In other embodiments, the intervening polypeptide is a fluorescent protein selected from the group consisting of the mHoneydew, mBanana, mOrange, dTomato, tdTomato, mTangerine, mStrawberry, mCherry, mGrapel, mRaspberry, mGrape2, mPlum [Shaner et al. (2005) Nat. Methods 2:905-909], and the like.

In a preferred embodiment, the fluorescent protein of the intervening region of the fusion protein of the invention is GFP (SEQ ID NO: 35).

“Albumin”, as used herein, refers to the water-soluble, non-glycosylated globular proteins commonly found in the plasma of animals, especially mammals. In a preferred embodiment, the protein of the intervening region of the fusion protein is albumin, more preferably human albumin (SEQ ID NO: 36).

“Nidogen”, as used herein, relates to any protein of the family of nidogens, formerly known as entactins, which are sulfated monomeric glycoproteins located in the basal lamina.

In a more preferred embodiment of the invention, the protein of the intervening region of the fusion protein is selected from the group consisting of the human nidogen 1 (NID1, SEQ ID NO: 37) and the human nidogen 2 (NID2, SEQ ID NO: 38).

“Chorionic gonadotropin” (GC), as used herein, refers to the glycoprotein and hormone produced in the placenta of mammals after the zygote implantation. Human chorionic gonadotropin presents two subunits, alpha (a) and beta (B). It is intended that any one of the two subunits separately and both together are suitable for the purposes of the invention.

Thus, in another embodiment of the invention, the protein of the intervening region of the fusion protein is the chorionic gonadotropin.

In another preferred embodiment of the invention, the protein of the intervening region of the fusion protein is the human chorionic gonadotropin (hGC, SEQ ID NO: 39).

As used herein, the term cystatin refers to a member of a family of protease inhibitors known as cystatins which are capable of inhibiting the activity of peptidase enzymes belonging to peptidase families C1 (papain family) and C13 (legumain family). In a preferred embodiment, the cystatin is selected from the group consisting of cystatin A, cystatin B, cystatin C, cystatin D and cystatin M. In yet another preferred embodiment, the cystatin is a cystatin A, also known as Stefin A. In a preferred embodiment, Stefin A is of human origin having the sequence SEQ ID NO:40. In yet another embodiment, the cystatin is a stefin A variant having one or more mutations selected from the group consisting of the G4W, the G4R, the V48D, the V48L, the G50S, the K71N, the S72G, the L73P, the L82R, the T83S mutations. In other embodiments, the stefin A variant contains the following mutations with respect to the sequence shown in SEQ ID NO:40:
G4W, V48D, K71N, S72G and L73P, corresponding to the mutant defined as STM mutant in Woodward et al. (J. Mol. Biol. (2005) 352, 1118-1133) and in Hoffman et al., Protein Engineering, Design & Selection vol. 23 no. 5 pp. 403-413, 2010).
G4R, V48L, G50S, K71N, S72G, L73P, L82R and T83S, corresponding to the mutant defined as SQM mutant in Hoffman et al., supra.) and in.
G4R corresponding to the mutant defined as SUN mutant in Hoffman et al., supra.).
V48L and G50S corresponding to the mutant defined as SUM mutant in Hoffman et al., supra.).
K71N, S72G, L73P, L82R and T83S corresponding to the mutant defined as SUC mutant in Hoffman et al., supra.).
V48L, G50S, K71N, S72G, L73P, L82R and T83S, corresponding to the mutant defined as SDM mutant in Hoffman et al., supra.).

Other suitable polypeptides and proteins that can be used as components of the intervening region include any polypeptide or protein without any physiological or biological activity on their own, as well as any biologically non-reactive peptide or protein.

In another embodiment of the invention the protein of the intervening region of the fusion protein is an inert protein.

As used herein, “Inert protein” refers to polypeptides or proteins or fragments or domains of proteins without known physiological or biological activity, or without the ability to specifically interact with other macromolecules for a biological function, and fragments or domains of proteins devoid of known therapeutic activity (e.g. antitumor activity). The inert protein that is part of the fusion protein is non-reactive and functions as a physical structure for the binding of the therapeutic agents. It is intended that the inert proteins do not comprise any motifs that have intrinsic enzymatic, physiological, or biological activity on their own, nor do they present immune reactivity, meaning that they stimulate neither the adaptive, nor the innate immune responses.

In general, wherein the protein of the intervening region of the fusion protein is concerned, it is intended that any intrinsic activity of said protein is irrelevant for the purposes of the invention and does neither contribute, nor hinder the biological activity of the therapeutic agent.

In a particular embodiment, the intervening polypeptide of the fusion proteins of the invention is a fragment of any of the proteins described in any of the embodiments of this section D.

In another preferred embodiment, the intervening polypeptide of the fusion protein of the invention is a mutant of any of the proteins described in any of the embodiments of this section D.

### E. The Therapeutic Agent

The term “therapeutic agent”, as used herein, is drawn to any compound, without chemical structure limitations, suitable for therapy and/or treatment of a condition, disorder or disease.

The nature of the therapeutic agent is not particularly limiting for the present invention provided it remains active in the fusion protein or can be activated once it is delivered to the inside of the cell. Accordingly, any therapeutic agent can be used in the fusion protein provided that it shows an activity or can reach an activity once it is delivered to the inside of the cell of at least 100%, at least 90%, at least 80%, at least 70%, at least 60%, at least 50% or less of the activity of the unconjugated therapeutic agent. Alternatively, since the purpose of the invention is to facilitate the action of the therapeutic agent by increasing its selectivity and reducing its off-target effects, it is contemplated that the effects of the therapeutic agent conjugated to the fusion protein may be synergic and exceed the parametrized values already known for the specific therapeutic agent. Accordingly, it is intended that some embodiments of the therapeutic agent conjugated to the fusion protein of the invention also show at least 101%, at least 105%, at least 110%, at least 115%, at least 120%, at least 125%, at least 130%, at least 135%, at least 140%, at least 145%, at least 150%, at least 175%, at least 200%, at least 300%, at least 400%, at least 500%, at least 1000%, or more of the functionality of the therapeutic agent alone.

In an embodiment of the invention, the therapeutic agent of the intervening region of the fusion protein of the invention is selected from the group consisting of
(i) a chemotherapy agent,
(ii) a cytotoxic polypeptide,
(ii) an antiangiogenic polypeptide,
(iii) a polypeptide encoded by a tumor suppressor gene,
(iv) a pro-apoptotic polypeptide,
(v) a polypeptide having anti-metastatic activity,
(vi) a polypeptide encoded by a polynucleotide which is capable of activating the immune response towards a tumor,
(vii) an antiangiogenic molecule, and
(viii) a toxin.

### (i) Chemotherapy Agent

It will be understood that the term “chemotherapeutic agents” refers to anti-cancer agents.

As used herein, an anti-cancer agent is an agent that at least partially inhibits the development or progression of a cancer, including inhibiting in whole or in part symptoms associated with the cancer even if only for the short term.

Several anti-cancer agents can be categorized as DNA damaging agents and these include topoisomerase inhibitors (e.g., etoposide, ramptothecin, topotecan, teniposide, mitoxantrone), DNA alkylating agents (e.g., cisplatin, mechlorethamine, cyclophosphamide, ifosfamide, melphalan, chorambucil, busulfan, thiotepa, carmustine, lomustine, carboplatin, dacarbazine, procarbazine), DNA strand break inducing agents (e.g., bleomycin, doxorubicin, daunorubicin, idarubicin, mitomycin C), anti-microtubule agents (e.g., vincristine, vinblastine), anti-metabolic agents (e.g., cytarabine, methotrexate, hydroxyurea, 5-fluorouracil, floxuridine, 6-thioguanine, 6-mercaptopurine, fludarabine, pentostatin, chlorodeoxyadenosine), anthracyclines, vinca alkaloids, or epipodophyllotoxins.

Additional examples of anti-cancer agents include without limitation Acivicin; Aclarubicin; Acodazole Hydrochloride; Acronine; Adozelesin; Aldesleukin; Altretamine; Ambomycin; Ametantrone Acetate; Aminoglutethimide; Amsacrine, Anastrozole; Anthramycin; Asparaginase; Asperlin; Azacitidine; Azetepa; Azotomycin; Batimastat; Benzodepa; Bicalutamide; Bisantrene Hydrochloride; Bisnafide Dimesylate; Bizelesin; Bleomycin Sulfate; Bortezomib (VELCADE); Brequinar Sodium; Bropirimine; Busulfan; Cactinomycin; Calusterone; Caracemide; Carbetimer; Carboplatin (a platinum-containing regimen); Carmustine; Carubicin Hydrochloride; Carzelesin; Cedefingol; Chlorambucil; Cirolemycin; Cisplatin (a platinum-containing regimen); Cladribine; Crisnatol Mesylate; Cyclophosphamide; Cytarabine; Dacarbazine; Dactinomycin; Daunorubicin; Decitabine; Dexormaplatin; Dezaguanine; Diaziquone; Docetaxel (TAXOTERE); Doxorubicin; Droloxifene; Dromostanolone; Duazomycin; Edatrexate; Eflornithine; Elsamitrucin; Enloplatin; Enpromate; Epipropidine; Epirubicin; Erbulozole; Erlotinib (TARCEVA), Esorubicin; Estramustine; Etanidazole; Etoposide; Etoprine; Fadrozole; Fazarabine; Fenretinide; Floxuridine; Fludarabine; 5-Fluorouracil; Flurocitabine; Fosquidone; Fostriecin; Gefitinib (IRESSA), Gemcitabine; Hydroxyurea; Idarubicin; Ifosfamide; Ilmofosine; Imatinib mesylate (GLEEVAC); Interferon alpha-2a; Interferon alpha-2b; Interferon alpha-n1; Interferon alpha-n3; Interferon beta-I a; Interferon gamma-I b; Iproplatin; Irinotecan; Lanreotide; Lenalidomide (REVLLMID, REVIMID); Letrozole; Leuprolide; Liarozole; Lometrexol; Lomustine; Losoxantrone; Masoprocol; Maytansine; Mechlorethamine; Megestrol; Melengestrol; Melphalan; Menogaril; Mercaptopurine; Methotrexate; Metoprine; Meturedepa; Mitindomide; Mitocarcin; Mitocromin; Mitogillin; Mitomalcin; Mitomycin; Mitosper; Mitotane; Mitoxantrone; Mycophenolic Acid; Nocodazole; Nogalamycin; Ormaplatin; Oxisuran; Paclitaxel; Pemetrexed (ALIMTA), Pegaspargase; Peliomycin; Pentamustine; Pentomone; Peplomycin; Perfosfamide; Pipobroman; Piposulfan; Piritrexim Isethionate; Piroxantrone; Plicamycin; Plomestane; Porfimer; Porfiromycin; Prednimustine; Procarbazine; Puromycin; Pyrazofurin; Riboprine; Rogletimide; Safingol; Semustine; Simtrazene; Sitogluside; Sparfosate; Sparsomycin; Spirogermanium; Spiromustine; Spiroplatin; Streptonigrin; Streptozocin; Sulofenur; Talisomycin; Tamsulosin; Taxol; Taxotere; Tecogalan; Tegafur; Teloxantrone; Temoporfin; Temozolomide (TEMODAR); Teniposide; Teroxirone; Testolactone; Thalidomide (THALOMID) and derivatives thereof; Thiamiprine; Thioguanine; Thiotepa; Tiazofurin; Tirapazamine; Topotecan; Toremifene; Trestolone; Triciribine; Trimetrexate; Triptorelin; Tubulozole; Uracil; Mustard; Uredepa; Vapreotide; Verteporfin; Vinblastine; Vincristine; Vindesine; Vinepidine; Vinglycinate; Vinleurosine; Vinorelbine; Vinrosidine; Vinzolidine, Vorozole; Zeniplatin; Zinostatin; Zorubicin.

In one embodiment, the anti-cancer agent is provided as an oligomer containing several units of the anti-cancer molecule. In one embodiment, the anti-cancer agent is a floxuridin poly- or oligonucleotide, which comprises several floxuridine molecules. The floxuridine poly- or poligonucleotide contains at least 2, 3, 4, 5, 6, 7, 8, 9, 10, 15, 20, 25, 30, 35, 40, 45, 50 or more floxuridine molecules. In a preferred embodiment the floxuridine polynucleotide is a floxuridine pentanucleotide, i.e. a oligonucleotide containing 5 floxuridine molecules.

The anti-cancer agent may be an enzyme inhibitor including without limitation tyrosine kinase inhibitor, a CDK inhibitor, a MAP kinase inhibitor, or an EGFR inhibitor. The tyrosine kinase inhibitor may be without limitation Genistein (4′, 5, 7-trihydroxyisoflavone), Tyrphostin 25 (3,4,5-trihydroxyphenyl), methylene]-propanedinitrile, Herbimycin A, Daidzein (4′,7-dihydroxyisoflavone), AG-126, trans-1-(3′-carboxy-4′-hydroxyphenyl)-2-(2″,5″-dihydroxy-phenyl) ethane, or HDBA (2-Hydroxy5-(2,5-Dihydroxybenzylamino)-2-hydroxybenzoic acid. The CDK inhibitor may be without limitation p21, p27, p57, p15, p16, p18, or p19. The MAP kinase inhibitor may be without limitation KY12420 (C23H24O8), CNI-1493, PD98059, or 4-(4-Fluorophenyl)-2-(4-methylsulfinyl phenyl)-5-(4-pyridyl) 1H-imidazole. The EGFR inhibitor may be without limitation erlotinib (TARCEVA), gefitinib (IRESSA), WHI-P97 (quinazoline derivative), LFM-A12 (leflunomide metabolite analog), ABX-EGF, lapatinib, canertinib, ZD-6474 (ZACTIMA), AEE788, and AG1458.

The anti-cancer agent may be a VEGF inhibitor including without limitation bevacizumab (AVASTIN), ranibizumab (LUCENTIS), pegaptanib (MACUGEN), sorafenib, sunitinib (SUTENT), vatalanib, ZD-6474 (ZACTIMA), anecortave (RETAANE), squalamine lactate, and semaphorin. The anti-cancer agent may be an antibody or an antibody fragment including without limitation an antibody or an antibody fragment including but not limited to bevacizumab (AVASTIN), trastuzumab (HERCEPTIN), alemtuzumab (CAMPATH, indicated for B cell chronic lymphocytic leukemia,), gemtuzumab (MYLOTARG, hP67.6, anti-CD33, indicated for leukemia such as acute myeloid leukemia), rituximab (RITUXAN), tositumomab (BEXXAR, anti-CD20, indicated for B cell malignancy), MDX-210 (bispecific antibody that binds simultaneously to HER-2/neu oncogene protein product and type I Fc receptors for immunoglobulin G (IgG) (Fc gamma RI)), oregovomab (OVAREX, indicated for ovarian cancer), edrecolomab (PANOREX), daclizumab (ZENAPAX), palivizumab (SYNAGIS, indicated for respiratory conditions such as RSV infection), ibritumomab tiuxetan (ZEVALIN, indicated for Non-Hodgkin's lymphoma), cetuximab (ERBITUX), MDX-447, MDX-22, MDX-220 (anti-TAG-72), I0R-C5, 10R-T6 (anti-CD 1), IOR EGF/R3, celogovab (ONCOSCINT OV 103), epratuzumab (LYMPHOCIDE), pemtumomab (THERAGYN), and Gliomab-H (indicated for brain cancer, melanoma).

It is contemplated that in certain embodiments of the invention a protein that acts as an angiogenesis inhibitor is targeted to a tumor. These agents include, in addition to the anti-angiogenic polypeptides mentioned above, Marimastat; AG3340; COL-3, BMS-275291, Thalidomide, Endostatin, SU5416, SU6668, EMD121974, 2-methoxyoestradiol, carboxiamidotriazole, CMlOl, pentosan polysulphate, angiopoietin 2 (Regeneron), herbimycin A, PNU145156E, 16K prolactin fragment, Linomide, thalidomide, pentoxifylline, genistein, TNP470, endostatin, paclitaxel, accutin, angiostatin, cidofovir, vincristine, bleomycin, AGM-1470, platelet factor 4 or minocycline.

Other suitable active agents are DNA cleaving agents. Examples of DNA cleaving agents suitable for inclusion as the cell toxin in the conjugates used in practicing the methods include, but are not limited to, anthraquinone-oligopyrrol-carboxamide, benzimidazole, leinamycin; dynemycin A; enediyne; as well as biologically active analogs or derivatives thereof (i.e., those having a substantially equivalent biological activity). Known analogs and derivatives are disclosed, for examples in Islam et al., J. Med. Chem. 34 2954-61, 1991; Skibo et al., J. Med. Chem. 37:78-92, 1994; Behroozi et al., Biochemistry 35:1568-74, 1996; Helissey et al., Anticancer Drug Res. 11:527-51, 1996; Unno et al., Chem. Pharm. Bull. 45:125-33, 1997; Unno et al., Bioorg. Med. Chem., 5:903-19, 1997; Unno et al., Bioorg. Med. Chem., 5:883-901, 1997; and Xu et al., Biochemistry 37:1890-7, 1998). Other examples include, but are not limited to, endiyne quinone imines (U.S. Pat. No. 5,622,958); 2,2r-bis(2-aminoethyl)-4-4′-bithiazole [Lee et al., Biochem. Mol. Biol. Int. 40:151-7, 1996]; epilliticine-salen.copper conjugates [Routier et al., Bioconjug. Chem., 8:789-92, 1997].

Some of the aforementioned chemotherapy agents can be grouped together under a common category as antimetabolites. “Antimetabolite” as used herein, refers to the compounds which inhibit the use of a metabolite that is part of normal metabolism. Antimetabolites are often similar in structure to the metabolite that they interfere with, such as the antifolates that interfere with the use of folic acid. Non-limiting examples of antimetabolites include the following compounds: bleomycin, busulfan, capecitabine, carmustine, carboplatin, chlorodeoxyadenosine, cisplatin, cyclophosphamide, cytarabine, dacarbazine, daunorubicin, docetaxel, doxorubicin, etoposide, fludarabine, fluorouracil, gemcitabine, hydroxyurea, idarubicin, ifosfamide, irinotecan, lomustine, melphalan, mercaptopurine, methotrexate mitomycin, mitoxantrone, oxaliplatin, paclitaxel, procarbazine, SN-38, thioguanine, thiotepa, teniposide vinblastine, vincristine, and vinorelbine.

### (ii) Cytotoxic Polypeptides

As used herein, the term “cytotoxic polypeptide” refers to an agent that is capable of inhibiting cell function. The agent may inhibit proliferation or may be toxic to cells. Any polypeptides that when internalized by a cell interfere with or detrimentally alter cellular metabolism or in any manner inhibit cell growth or proliferation are included within the ambit of this term, including, but not limited to, agents whose toxic effects are mediated when transported into the cell and also those whose toxic effects are mediated at the cell surface. Useful cytotoxic polypeptides include proteinaceous toxins such as bacterial toxins.

Examples of proteinaceous cell toxins useful for incorporation into the conjugates according to the invention include, but are not limited to, type one and type two ribosome inactivating proteins (RIP). Useful type one plant RIPs include, but are not limited to, dianthin 30, dianthin 32, lychnin, saporins 1-9, pokeweed activated protein (PAP), PAP II, PAP-R, PAP-S, PAP-C, mapalmin, dodecandrin, bryodin-L, bryodin, Colicin 1 and 2, luffin-A, luffin-B, luffin-S, 19K-protein synthesis inhibitory protein (PSI), 15K-PSI, 9K-PSI, alpha-kirilowin, beta-kirilowin, gelonin, momordin, momordin-II, momordin-Ic, MAP-30, alpha-momorcharin, beta-momorcharin, trichosanthin, TAP-29, trichokirin; barley RIP; flax RIP, tritin, corn RIP, Asparin 1 and 2 [Stirpe et al., 1992. Bio/Technology 10:405-12]. Useful type two RIPs include, but are not limited to, volkensin, ricin, nigrin-b, CIP-29, abrin, modeccin, ebulitin-[alpha], ebulitin-[beta], ebultin-[gamma], vircumin, porrectin, as well as the biologically active enzymatic subunits thereof [Stirpe et al., 1992. Bio/Technology 10:405-12; Pastan et al., 1992. Annu. Rev. Biochem. 61:331-54; Brinkmann and Pastan, 1994. Biochim. et Biophys. Acta 1198:27-45; and Sandvig and Van Deurs, 1996. Physiol. Rev. 76:949-66].

Examples of bacterial toxins useful as cell toxins include, but are not limited to, shiga toxin and shiga-like toxins (i.e., toxins that have the same activity or structure), as well as the catalytic subunits and biologically functional fragments thereof. These bacterial toxins are also type two RIPs [Sandvig and Van Deurs, 1996. Physiol. Rev. 76:949-66; Armstrong, 1995. J. Infect. Dis., 171:1042-5; Kim et al., 1997. Microbiol. Immunol. 41:805-8; and Skinner et al., 1998. Microb. Pathog. 24:117-22]. Additional examples of useful bacterial toxins include, but are not limited to, Pseudomonas exotoxin and Diphtheria toxin [Pastan et al., 1992. Annu. Rev. Biochem. 61:331-54; and Brinkmann and Pastan, 1994. Biochim. et Biophys. Acta 1198:27-45]. Truncated forms and mutants of the toxin enzymatic subunits also can be used as a cell toxin moiety (Pastan et al., Annu. Rev. Biochem. 61:331-54; Brinkmann and Pastan, Biochim. et Biophys. Acta 1198:27-45, 1994; Mesri et al., J. Biol. Chem. 268:4852-62, 1993; Skinner et al., Microb. Pathog. 24:117-22, 1998; and U.S. Pat. No. 5,082,927). Other targeted agents include, but are not limited to the more than 34 described Colicin family of RNase toxins which include colicins A, B, D, E1-9, cloacin DF13 and the fungal RNase, [alpha]-sarcin [Ogawa et al. 1999. Science 283:2097-100; Smarda et al., 1998. Folia Microbiol (Praha) 43:563-82; Wool et al., 1992. Trends Biochem. Sci., 17:266-69].

(iii) Antiangiogenic Polypeptides

Proliferation of tumor cells relies heavily on extensive tumor vascularization, which accompanies cancer progression. Thus, inhibition of new blood vessel formation with anti-angiogenic agents and targeted destruction of existing blood vessels have been introduced as effective and relatively non-toxic approaches to tumor treatment.

The term “anti-angiogenic polypeptide”, as used herein, denotes a polypeptide capable of inhibiting angiogenesis. Suitable antiangiogenic polypeptides include, without limitation, angiostatin, endostatin, anti-angiogenic anti-thrombin III, sFRP-4 as described in WO2007115376, and an anti-VEGF antibody such as anibizumab, bevacizumab (avastin), Fab IMC 1121 and F200 Fab.

### (iv) Polypeptides Encoded by a Tumor Suppressor Gene

As used herein, a “tumor suppressor” is a gene or gene product that has a normal biological role of restraining unregulated growth of a cell. The functional counterpart to a tumor suppressor is an oncogene—genes that promote normal cell growth may be known as “proto-oncogenes” A mutation that activates such a gene or gene product further converts it to an “oncogene”, which continues the cell growth activity, but in a dysregulated manner Examples of tumor suppressor genes and gene products are well known in the literature and may include PTC, BRCA1, BRCA2, p16, APC, RB, WT1, EXT1, p53, NF1, TSC2, NF2, VHL, ST7, ST14, PTEN, APC, CD95 or SPARC.

### (v) Pro-Apoptotic Polypeptides

The term “pro-apoptotic polypeptides”, as used herein, refers to a protein which is capable of inducing cell death in a cell or cell population. The overexpression of these proteins involved in apoptosis displaces the careful balance between anti-apoptotic and pro-apoptotic factors towards an apoptotic outcome. Suitable pro-apoptotic polypeptides include, without limitation, pro-apoptotic members of the BCL-2 family of proteins such as BAX, BAK, BOK/MTD, BID, BAD, BIK/NBK, BLK, HRK, BIM/BOD, BNIP3, NIX, NOXA, PUMA, BMF, EGL-I, and viral homologs, caspases such as caspase-8, the adenovirus F4orf4 gene, p53 pathway genes, pro-apoptotic ligands such as TNF, FasL, TRAIL and/or their receptors, such as TNFR, Fas, TRAIL-R1 and TRAIL-R2.

(vi) Polypeptides with Anti-Metastatic Activity

The term “metastasis suppressor” as used herein, refers to a protein that acts to slow or prevent metastases (secondary tumors) from spreading in the body of an organism with cancer. Suitable metastasis suppressor include, without limitation, proteins such as BRMS1, CRSP3, DRG1, KAI1, KISS-1, NM23, a TIMP-family protein and uteroglobin.

(vii) Polypeptides Encoded by a Polynucleotide Capable of Activating the Immune Response Towards a Tumor

As used herein, an immunostimulatory polypeptide agent is a polypeptide encoded by a polynucleotide which is capable of activating or stimulating the immune response (including enhancing a pre-existing immune response) in a subject to whom it is administered, whether alone or in combination with another agent. Suitable non-limiting examples of immunostimulatory peptides include flagellin, muramyl dipeptide), cytokines including interleukins (e.g., IL-2, IL-7, IL-15 (or superagonist/mutant forms of these cytokines), IL-12, IFN-gamma, IFN-alpha, GM-CSF, FLT3-ligand, etc.), immunostimulatory antibodies (e.g., anti-CTLA-4, anti-CD28, anti-CD3, or single chain/antibody fragments of these molecules), and the like.

(viii) Antiangiogenic Molecules

It is also contemplated that in certain embodiments the intervening region of the fusion protein of the invention corresponds to a protein that acts as an angiogenesis inhibitor is targeted to a tumor. These agents include, in addition to the anti-angiogenic polypeptides mentioned above, Marimastat; AG3340; COL-3, BMS-275291, Thalidomide, Endostatin, SU5416, SU6668, EMD121974, 2-methoxyoestradiol, carboxiamidotriazole, CMlOl, pentosan polysulphate, angiopoietin 2 (Regeneron), herbimycin A, PNU145156E, 16K prolactin fragment, Linomide, thalidomide, pentoxifylline, genistein, TNP470, endostatin, paclitaxel, accutin, angiostatin, cidofovir, vincristine, bleomycin, AGM-1470, platelet factor 4 or minocycline. Also included are VEGF inhibitors including without limitation bevacizumab (AVASTIN), ranibizumab (LUCENTIS), pegaptanib (MACUGEN), sorafenib, sunitinib (SUTENT), vatalanib, ZD-6474 (ZACTIMA), anecortave (RETAANE), squalamine lactate, and semaphorin.

### (ix) Toxins

As used herein, the term “toxins” refers to non-proteinaceous/non-polypeptidic cytotoxic compounds obtained from different organisms, as well as chemically modified derivatives of those same compounds and compounds obtained through chemical synthesis. The compounds of this category with biological origin may be obtained from microorganisms (whether bacteria, archaea, protozoa or unicellular fungi) or pluricellular organisms (pluricellular fungi, plants, or animals, like mollusks). It is intended that the chemical composition and structure of these toxins is not limited in any way beyond their non-polypeptidic nature, therefore one or more amino acids may be part of their structure, whether as part of their basic composition or as result of chemical derivation, as long as all the amino acids participating in the structure are not bound together by peptide bonds.

Examples of toxins suitable for the invention are calicheamycin γ1, dolastatin 10, maytansinoid (DM1) and pyrrolobenzodiazepine dimer (PBD).

### F. Linkage of the Therapeutic Agent to the Intervening Region

The therapeutic agent of the invention is conjugated to the fusion protein of the invention. It is intended that the therapeutic agent, as aforementioned, is conjugated to the intervening region of the fusion protein without limitation of the position of the conjugation inside the intervening region with regards to the N-terminal and C-terminal ends. Accordingly, the therapeutic agent can be conjugated to the intervening polypeptide region in an equidistant position with respect to the N-terminal and C-terminal ends or it can be closer to either of them. Hence, the therapeutic agent can be conjugated to the intervening polypeptide region at a distance of 100, 90, 80, 75, 70, 65, 60, 55, 50, 45, 40, 35, 30, 25, 30, 25, 20, 15, 20, 10, or less amino acid residues from the N-terminal or C-terminal end, or at the same residue of the N-terminal or C-terminal end.

The only intended limitation in the conjugation position of the therapeutic agent is that the therapeutic agent and the elements of the fusion protein are functional and the conjugation of the therapeutic agent does not interfere with the activity of either therapeutic agent or the fusion protein. So, the therapeutic agent, the polycationic peptide, and the positively charged amino acid-rich region conserve at least 45%, at least 50%, at least 55%, at least 60%, at least 65%, at least 70%, at least 75%, at least 80%, at least 85%, at least 90%, preferably 95%, more preferably 99%, even more preferably 100% of their functionality with respect to the non-conjugated forms of the fusion protein and the therapeutic agent respectively.

It is intended that the therapeutic agent can either be conjugated directly to a residue of the intervening polypeptide of the fusion protein or its bond may be mediated by a linking moiety. “Linking moiety” as used herein, relates to a molecule connecting the therapeutic peptide to the intervening region of the fusion protein. It is also intended that the linking moiety is not limited in its chemical nature and/or structure; therefore, the linking moiety may be a polysaccharide, a polypeptide, a fatty acid, a phospholipid, or a chemical derivative thereof, among others. It is further intended that the therapeutic agent may be bound to the linking moiety through any chemical bond, such as peptide bond, isopeptide bond, amide bond, imine bond, and etcetera.

The person skilled in the art will acknowledge that the previous provisions regarding the functionality of the elements of the fusion protein and the therapeutic agent apply also whenever a coupler mediates the conjugation between the therapeutic agent and the fusion protein. Therefore, whenever the therapeutic agent is conjugated to the fusion protein through a linking moiety, the therapeutic agent, the polycationic peptide, and the positively charged amino acid-rich region conserve at least 45%, at least 50%, at least 55%, at least 60%, at least 65%, at least 70%, at least 75%, at least 80%, at least 85%, at least 90%, preferably 95%, more preferably 99%, even more preferably 100% of their functionality with respect to the non-conjugated forms of the fusion protein and the therapeutic agent respectively, regardless of the position of the conjugation in the intervening region, the chemical composition or structure of the linking moiety and the chemical nature of the bond/s between the linking moiety and the therapeutic agent and between the linking moiety and the intervening region.

Thus, in an embodiment of the invention, the therapeutic agent is bound to the intervening polypeptide region of the fusion protein directly.

In another embodiment of the invention, the therapeutic agent is bound to the intervening region of the fusion protein though a linking moiety.

In a preferred embodiment of the invention, the linking moiety that mediates the bond of the therapeutic agent to the intervening region of the fusion protein through is 6-Maleimidohexanoic acid N-hydroxysuccinimide ester. In another preferred embodiment, the linking moiety that mediates the bond of the therapeutic agent to the intervening region of the fusion protein through is 4-Maleimido hexanoic acid N-hydroxysuccinimide ester.

In some embodiments of the invention, the linking moiety binding the therapeutic agent to the intervening region of the fusion protein is susceptible to be processed by enzymes present in the cytoplasm, releasing the therapeutic agent from the fusion protein once the therapy agent conjugated to the fusion protein has been internalized in a cell.

As the person skilled in the art will recognize, the numerous residues of the polypeptidic chain of the intervening region of the fusion protein offer not only multiple positions wherein a therapeutic agent can be bound or linked but also the possibility of linking more than one molecule of the same or different therapeutic agents to the same fusion protein. As previously, it is intended that the provisions regarding the functionality of the elements of the fusion protein as well as the therapeutic agent/s are upheld and that the increase of the number of molecules of the same or of a different therapeutic agent, the binding of different therapeutic agents, their chemical nature, or their binding positions do not affect the effectiveness and functionality of each therapeutic agent.

Besides, some therapeutic agents may be polymerized in such a way that multiple copies of the same molecule may be bound together like polymers of analogs of nucleotides can oligonucleotides, for instance 5-Fluoro-2′-deoxyUridine (FdU), which result in oligo-FdU. It is intended that some embodiments of the invention may comprise such polymers. Also, it is intended that some other embodiments of the invention may comprise polymers of 2 or more different molecules of therapeutic agents provided that the therapeutic agents do not interfere with the physiological or biological effects of each other. The person skilled in the art will recognize that those embodiments of the invention featuring polymers of therapeutic agents may feature 2, 3, 4, 5, 6, 7, 8, 9, 10, 15, 20, 15, 30, 40, 50 or more molecules polymerized together of 1 or more different therapeutic agents in a proportion of 1:1, 1:2, 1:3, 1:4, 1:5, 1:6, 1:7, 1:8, 1:9, 1:10, or more.

Thus, in another embodiment of the invention, the intervening polypeptide of the fusion protein is conjugated to a plurality of therapeutic agents, wherein said plurality of therapeutic agents are the same or different.

In a preferred embodiment of the invention, the therapeutic agent conjugated to the intervening polypeptide region of the fusion protein is a chemotherapy agent.

In a more preferred embodiment of the invention, the chemotherapy agent conjugated to the intervening region of the fusion protein is an antimetabolite.

In an even more preferred embodiment of the invention, the chemotherapy agent conjugated to the intervening region of the fusion protein is an antimetabolite.

In an even yet more preferred embodiment of the invention, the antimetabolite conjugated to the intervening region of the fusion protein is a pyrimidine analogue or an oligomeric form thereof.

In a most preferred embodiment of the invention, the pyrimidine analogue conjugated to the intervening region of the fusion protein is floxuridine.

### G. Reporter Proteins

In another embodiment of the invention, the fusion protein of the invention further comprises a reporter protein.

The person skilled in the art will acknowledge the term “reporter protein” as referring to a protein resulting from the expression of a “reporter gene”. Reporter proteins are well known and commonly used in the art as markers suitable for multiple purposes, such as location of the expression of the reporter genes in tissues, cells or subcellular locations, protein-protein interactions, transport across the plasmatic membranes or endomembranes, vesicular traffic, ligand-receptor interactions, etcetera.

Useful reporter proteins in the context of the present invention include luciferase-4-monooxygenase from Photinus pyralis, β-galactosidase, thymidine kinase, and the like. The reporter proteins also include fluorescent proteins, which have already been discussed.

The reporter protein comprised by the fusion protein of the invention is directly adjacent to the positively charged amino acid-rich region or separated by a linker. The relative position of the positively charged amino acid-rich region, however, remains as per the the aforementioned considerations about the relative position of the elements of the fusion protein. Hence, independently of the position of the fusion protein, the fluorescent protein is always adjacent to it, either directly or separated by a linker.

Accordingly, in the embodiments of the invention comprising a fluorescent protein, the possible relative positions of the elements of the fusion protein of the invention would fit the following scheme (wherein RP refers to a reporter protein and the numbering stated above for the elements is retained: (1) polycationic peptide, (2) intervening polypeptide region, (3) positively charged amino acid region):
N-(1)-(2)-RP-(3)-C
N-(1)-linker-(2)-RP-(3)-C
N-(1)-(2)-linker-RP-(3)-C
N-(1)-linker-(2)-linker-RP-(3)-C
N-(3)-RP-(2)-(1)-C
N-(3)-RP-linker-(2)-(1)-C
N-(3)-RP-(2)-linker-(1)-C
N-(3)-RP-linker-(2)-linker-(3)-C
N-(1)-(2)-RP-linker-(3)-C
N-(1)-linker-(2)-RP-linker-(3)-C
N-(1)-(2)-linker-RP-linker-(3)-C
N-(1)-linker-(2)-linker-RP-linker-(3)-C
N-(3)-linker-RP-(2)-(1)-C
N-(3)-linker-RP-linker-(2)-(1)-C
N-(3)-linker-RP-(2)-linker-(1)-C
N-(3)-linker-RP-linker-(2)-linker-(3)-C
N-(2)-(1)-RP-(3)-C
N-(2)-linker-(1)-RP-(3)-C
N-(2)-(1)-linker-RP-(3)-C
N-(2)-linker-(1)-linker-RP-(3)-C
N-(2)-RP-(3)-(1)-C
N-(2)-(3)-RP-(1)-C.
N-(2)-linker-RP-(3)-(1)-C
N-(2)-linker-(3)-RP-(1)-C
N-(2)-RP-(3)-linker-(1)-C
N-(2)-(3)-RP-linker-(1)-C
N-(2)-linker-RP-(3)-linker-(1)-C
N-(2)-linker-(3) RP-linker-(1)-C
N-(1)-RP-(3)-(2)-C
N-(1)-(3)-RP-(2)-C
N-(1)-RP-(3)-linker-(2)-C
N-(1)-(3)-RP-linker-(2)-C
N-(1)-linker-RP-(3)-(2)-C
N-(1)-linker-(3)-RP-(2)-C
N-(1)-linker-RP-(3)-linker-(2)-C
N-(1)-linker-(3)-RP-linker-(2)-C
N-RP-(3)-(1)-(2)-C
N-(3)-RP-(1)-(2)-C
N-RP-(3)-linker-(1)-(2)-C
N-(3)-RP-linker-(1)-(2)-C
N-RP-(3)-(1)-linker-(2)-C
N-(3)-RP-(1)-linker-(2)-C
N-RP-(3)-linker-(1)-linker-(2)-C
N-(3)-RP-linker-(1)-linker-(2)-C

### Preferred Fusion Proteins of the Invention

In a preferred embodiment, the fusion protein is the T22-GFP-H6-5FdU which comprises:
(i) The T22 peptide as polycationic peptide,
(ii) GFP as intervening polypeptide region,
(iii) A hexahistidine region as positively charged amino acid-rich region,
(iv) A floxuracile pentanucleotide as therapeutic agent

In a more preferred embodiment, the T22-GFP-H6-5FdU is formed by a linkage between an amino group in the side chain of the GFP protein and a thiol group in the therapeutic agent connected by a 6 atom spacer region.

In a preferred embodiment, the fusion protein is the T22-STM-H6-5FdU which comprises:
(i) The T22 peptide as polycationic peptide,
(ii) The STM variant of Ste2 as defined above as intervening polypeptide region,
(iii) A hexahistidine region as positively charged amino acid-rich region,
(iv) A floxuracile pentanucleotide as therapeutic agent.

In a more preferred embodiment, the T22-STM-H6-5FdU is formed by a linkage between an amino group in the side chain of the STM protein and a thiol group in the therapeutic agent connected by a 6 atom spacer region.

### Stoichiometry of the Fusion Protein and Nanoconjugates of the Invention

The number of therapeutic agents which are conjugated to the fusion protein of the invention, while not being particularly limitative, will depend on the number of available residues in the intervening polypeptide which are available for chemical conjugation with the therapeutic agent. Since most conjugations occur via amino- or sulfhydryl groups present in the side chains of the amino acids forming part of the intervening polypeptide, the number of therapeutic agents conjugated to the fusion protein will depend on the number of lysine and arginine residues (for a conjugation via an amino groups in the side chains) or on the number of cysteine residues (for conjugation via sulphidryl groups in the side chains) as well as on the yield of the conjugation reaction. Thus, in a particular embodiment of the invention, the fusion protein of the invention is conjugated to at least 1, 2, 3, 4, 5, 6, 7, 8, 9, 10, 12, 15, 17, 20, 25, 30 therapeutic agents.

It will be understood that, in the particular case wherein the therapeutic agent is provided as a polymer, the number of therapeutic agents will also depend on the number of the monomers in the polymer. In the particular case of a FdU oligomer, the number of therapeutic agents in a given fusion protein will be the result of multiplying the number of oligomers attached to the fusion protein by the number of monomers. In the preferred case of a FdU pentamer, preferred embodiments include fusion proteins comprising at least 5, 10, 15, 20, 25, 30, 35, 40, 45, 50, 60, 75, 85, 100, 125, 150 or more therapeutic agents per fusion protein, corresponding, respectively, to 1, 2, 3, 4, 5, 6, 7, 8, 9, 10, 12, 15, 17, 20, 25 or 30 FdU pentamers conjugated per molecule.

In addition, the nanoparticles according to the invention result from the assembly of multiple copies of the fusion proteins of the invention. In preferred embodiments, the nanoparticle comprises t least 2, 3, 4, 5, 6, 7, 8, 9, 10, 11, 12, 13, 14, 15, 17, 20, 25, more preferably at least 15 monomers of the fusion protein of the invention.

Accordingly, the total number of therapeutic agents attached to each nanoparticle will depend on (i) the number of therapeutic agents conjugated to each fusion protein, (ii) the oligomerization state of the therapeutic agents and (iii) the number of fusion proteins forming the nanoparticle. In preferred embodiments, the nanoparticle is conjugated to at least 30, 35, 40, 45, 50, 60, 65, 70, 57, 80, 85, 90, 59, 100, 125, 150, 175, 200, 225, 250, 275, 300 therapeutic agents. In a further preferred embodiment, the nanoparticle is conjugated to at least 30, 35, 40, 45, 50, 60, 65, 70, 57, 80, 85, 90, 59, 100, more preferably at least 60 molecules of FdU pentamer.

### Method for Preparing the Fusion Proteins of the Invention

In a second aspect, the invention relates to a method to prepare the fusion proteins of the invention comprising the steps of:
a) providing a fusion protein comprising
   i. a polycationic peptide,
   ii. an intervening polypeptide region and
   iii. a positively charged amino acid-rich region,
   wherein the polycationic peptide and the positively charged amino acid-rich region are located at the ends of the protein and
b) contacting said fusion protein with an activated form of a therapeutic agent or of an oligomeric form thereof wherein said activated form of a therapeutic agent or of an oligomeric form thereof contains a reactive group which is capable of reacting with at least one group in the intervening region of the fusion protein and wherein the contacting is carried out under conditions adequate for the formation of a bond between the reactive group in the therapeutic agent and the group in the intervening polypeptide region

In another embodiment, the invention relates to a method to prepare the fusion proteins of the invention comprising the steps of:
a) providing a fusion protein comprising
   i. a polycationic peptide,
   ii. an intervening polypeptide region and
   iii. a positively charged amino acid-rich region,
   wherein the polycationic peptide and the positively charged amino acid-rich region are located at the ends of the protein and wherein the fusion protein is provided in an activated form, wherein said activated form of the fusion protein contains a reactive group in the intervening region and
b) contacting said fusion protein with a therapeutic agent or an oligomeric form thereof, wherein said therapeutic agent contains a group which is capable of reacting with the reactive group in the fusion protein, wherein said contacting is carried out under conditions adequate for the formation of a bond between the reactive group in the fusion protein and the group in the therapeutic agent.

The person skilled in the art will recognize that “reactive group”, as used herein, refers to any moiety of a molecule which is capable of chemically reacting with another moiety from another molecule in such a fashion so as to bind the two molecules together, usually with the release of one or more additional molecules. Many such reactions are known in the art such as the formation of the peptide bond between a carboxyl and an amine group being one non-limiting example among them.

“Activated”, as used herein when referring to a molecule, refers to a modified version of the molecule which contains a chemical modification whereby said molecule is capable to chemically react in a manner not previously present in the molecule (for instance, the activation adds a moiety not present previously, allowing for a bond that was unfeasible before) or with an increased reactivity (meaning that the reaction of the molecule with another molecule requires a lower activation energy than in the inactivated state). The present invention contemplates the possibility of activating the therapeutic agent and then contacting the activated therapeutic agent with the fusion protein or of activating the fusion protein and then contacting the activated fusion protein with the therapeutic agent. In both cases, the activation of the fusion protein or of the therapeutic agent is usually carried out by reacting the molecule to be activated with a reagent that introduces the reactive group in a suitable moiety in the molecule to be activated. Examples of reactive groups that allow the therapeutic agent or fusion proteins to be activated include, but are not limited, to carboxyl, amine, imine, thiol, sulfone, hydroxyl, sulfate, and phosphate moieties, among many others which are commonly known to the person skilled in the art. The activated form of the therapeutic agent is also herein referred to as the “activated therapeutic agent”. The activated form of the fusion protein is also herein referred to as the “activated fusion protein”. The reactive group or groups in the activated fusion protein is or are located in the intervening region, although it is not excluded that additional reactive groups can also be found in other regions of the fusion protein.

In those embodiments of the invention wherein a linking moiety mediates the bond between the fusion protein and the therapeutic agent, the linking moiety is a bifunctional cross-linker and, more preferably, a heterobifunctional cross-linker, that reacts with the groups in the therapeutic agent and in the fusion protein, either sequentially (either reacting with the activated therapeutic agent first and then with the fusion protein, or first with the fusion protein and then with the activated therapeutic agent) or simultaneously, using among other linkages such as thioethers, amide bonds, carbon-nitrogen double bonds, or linkages generated by cycloaddition as disclosed in Kalia J et al. Advances in bioconjugation. Curr Org Chem 2010 Jan. 14 (2): 138-147). As a way of example typical thiol-reactive functional groups include iodoacetamides, maleimides, and disulfides. In addition, a protein can be treated with a small molecule or surface displaying an activated ester (e.g., an N-hydroxysuccinimidyl ester) to form amide bonds with the amino groups on lysine side chains and the N terminus. In another embodiment, the linking moiety is a heterobifunctional cross-linker which contains reactive groups capable of reacting with a thiol group and with an amino group. In one embodiment, the heterobifunctional cross-linker is 6-maleimidohexanoic acid N-hydroxysuccinimide ester.

In a preferred embodiment, the linking moiety reacts in a first step with the activated therapeutic agent and in a second step with the fusion protein. In another embodiment, the linking moiety reacts in a first step with the fusion protein and, in a second step, with the therapeutic agent.

It is intended that the step of contacting the fusion protein of the invention with the activated form of the therapeutic agent is carried out in a medium which favors the reaction establishing the bond between them. Media suitable for the reactions are commonly known to the person skilled in the art, including aqueous buffers and non-aqueous buffers. It is also intended that solid supports can be used in conjunction with the media for any of the reaction steps conducing to the synthesis of the activated therapeutic agent and the conjugate of the fusion protein, the therapeutic agent, and also the linking moiety in the embodiments that include one. Furthermore, it is intended that the method for the preparation of the conjugates between the fusion protein and the therapeutic agent is not limited to the fusion protein, the activated therapeutic agent, and the linking moiety, but that some embodiments include also the use of one or more catalysts and co-factors in the reaction.

Thus, in one embodiment of the invention, the activated form of the therapeutic agent contains a group which reacts with at least one of the side chains of a residue in a peptide region of the fusion protein, preferably in the intervening region of the fusion protein.

In another preferred embodiment said residue is an external lysine. In a further preferred embodiment of the invention, the group of the activated therapeutic agent, preferably the chemotherapeutic agent, which reacts with the side chain of the intervening region of the fusion protein is a thiol group.

In an even more preferred embodiment of the invention, the activated therapeutic agent is an activated chemotherapeutic agent, more preferably a thiol-functionalized oligo-floxuridine.

In a further preferred embodiment, the linking moiety is 4-maleimido hexanoic acid N-hydroxysuccinimide ester mediates the conjugation between the activated therapeutic agent and the side chain of the residue of the peptide region of the fusion protein indicated in the previous embodiments of this section. In a yet more preferred embodiment, the linking moiety 4-maleimido hexanoic acid N-hydroxysuccinimide ester is bound in a first step to the therapeutic agent, preferably the activated FdU, yet more preferably FdU functionalized with a sulfhydryl, and in a second step to the side chain in a residue of the fusion protein, more preferably to external lysines of the fusion protein, even more preferably to external lysines of the intervening region of the fusion protein.

It is also intended that the step of contacting the activated fusion protein of the invention with the therapeutic agent is carried out in a medium which favors the reaction establishing the bond between them. Media suitable for the reactions are commonly known to the person skilled in the art, including aqueous buffers and non-aqueous buffers. It is also intended that solid supports can be used in conjunction with the media for any of the reaction steps leading to the synthesis of the conjugate of the fusion protein and the therapeutic agent, and also the linking moiety in the embodiments that include one. Furthermore, it is intended that the method for the preparation of the conjugates between the fusion protein and the therapeutic agent is not limited to the fusion protein, the activated therapeutic agent, and the linking moiety, but that some embodiments include also the use of one or more catalysts and co-factors in the reaction.

Thus, in one embodiment of the invention, the activated form of the fusion protein contains a group which reacts with at least one moiety in the therapeutic agent. In a further preferred embodiment of the invention, the group of the therapeutic agent, preferably the chemotherapeutic agent, which reacts with the activated fusion protein is a thiol group.

In an even more preferred embodiment of the invention, the activated fusion protein agent is an amino functionalized fusion protein wherein one or more amino groups in the side chain of the amino acids forming part of the intervening polypeptide is modified with an activated group having thiol reactivity. In a further preferred embodiment, the linking moiety is 4-maleimido hexanoic acid N-hydroxysuccinimide ester mediates the conjugation between an amino group in the fusion protein and a thiol group in the therapeutic agent In a yet more preferred embodiment, the linking moiety 4-maleimido hexanoic acid N-hydroxysuccinimide ester is bound in a first step to the fusion protein, more preferably to external lysines of the fusion protein and in a second step to the therapeutic agent side chain in a residue of the fusion protein.

Nanoparticles of the Invention and Methods for Preparing them with the Fusion Proteins of the Invention

In a third aspect, the invention relates to a method to prepare nanoparticles comprising multiple copies of the fusion protein according to the first aspect of the invention comprising placing a preparation of said fusion protein in a low salt buffer.

As the person skilled in the art will recognize, “nanoparticles” are microscopic particles whose size is measured in nanometers. The nanoparticles of the invention comprise the nanoparticles that result from the aggregation of multiple copies of the fusion protein of the invention as defined in the previous section. In the method for preparing nanoparticles with the fusion proteins of the invention, the preparation of the fusion protein of the invention comprises the monomeric form of the fusion proteins of the invention, which are thermodynamically favored to form non-covalent electrostatic unions and spontaneously aggregate in the conditions of the low salt buffer.

The person skilled in the art will acknowledge that the size of the nanoparticles can be in the range between 1 and 1000 nm, more preferably between 2.5 and 500 nm, even more preferably between 5 and 250 nm, and yet even more preferably between 10 and 100 nm

It will be understood that the expression “low salt buffer” comprises any buffer solution resulting from the dissolution of one or more salts in water with the capability to moderate changes in pH, wherein the amount of dissolved salt or salts results in an osmolarity lower or equal to that of the physiological fluids, such as the cytoplasm or the extracellular medium, for instance. Thus, the low salt buffer is understood to keep pH and osmolarity inside the range of physiological values and will be used inside the range of physiological temperatures.

The person skilled in the art will recognize that the range of physiological temperatures can oscillate between 15 and 45° C., more preferably between 20 and 40° C., even more preferably between 25 and 39° C., yet even more preferably between 3° and 37° C. The person skilled in the art will also acknowledge that the osmolarity of the low salt buffer will be in the range between 100 and 400 milli-osmoles/L (mOsm/L), preferably between 150 and 350 mOsm/L, more preferably between 200 and 300 mOsm/L, even more preferably between 225 and 275 mOsm/L.

Low salt buffers suitable for the invention, for instance, are the Tris-dextrose buffer (20 mM Tris+5% dextrose, pH 7.4), the Tris-NaCl buffer (20 mM Tris, 500 NaCl, pH 7.4), the PBS-glycerol buffer (phosphate buffered saline, PBS, pH 7.4, which is well known in the art, +10% glycerol), Tris Buffered Saline (TBS)-dextrose (20 mM Tris-HCl buffer pH 7.5, well known in the art, 200NaCl, +5% dextrose), Tris Buffered Saline-Tween 20 (TBST) buffer (10 mM Tris-HCl pH 7.5, 200 mM NaCl, +0.01% Tween 20), or any physiological buffer known in the art with a pH not lower than 6

In a preferred embodiment of the invention, the low salt buffer of the method of the invention is selected from the group consisting of a carbonate buffer, a Tris buffer and a phosphate buffer.

In a particularly preferred embodiment of the invention, the low salt buffer of the method of the invention is a carbonate buffer that comprises sodium bicarbonate at a concentration between 100 and 300 nM. In another particularly preferred embodiment of the invention, the low salt buffer of the method of the invention is a Tris buffer that comprises Tris at a concentration of between 10 and 30 nM. In another particularly preferred embodiment of the method of the invention, the low salt buffer of the invention is a phosphate buffer that comprises Na₂HPO₄ and NaH₂PO₄ at a total concentration of between 5 mM and 20 mM.

In an even more preferred embodiment of the invention, the low salt buffer of the method of the invention further comprises dextrose and/or glycerol.

In a yet more preferred embodiment of the invention, the low salt buffer of the method of the invention has a pH between 6.5 and 8.5.

In an even yet more preferred embodiment of the invention, the low salt buffer of the method of the invention is selected from the group consisting of
(i) 166 mM NaHCO₃, pH 7.4
(ii) 20 mM Tris, 500 mM NaCl, 5% dextrose, pH 7.4
(iii) 140 mM NaCl, 7.5 mM Na₂HPO₄, 2.5 mM NaH₂PO₄, 10% glycerol, pH 7.4

In another aspect of the invention, the invention relates to nanoparticles comprising multiple copies of the fusion protein of the first aspect of the invention or prepared according to the method or the invention for preparing nanoparticles.

Thus, the nanoparticles of the invention comprise aggregates of multiple copies of the fusion proteins of the invention, which result from the electrostatic interaction between regions in their structures favoring their non-covalent binding and coupling in physiological conditions. Since the method of the invention for the preparation of nanoparticles comprises placing a preparation of the fusion protein of the invention in a low salt buffer, it is understood that the nanoparticles thus formed comprise also an aggregate of multiple copies of the fusion protein.

In a preferred embodiment of the invention, the nanoparticles of the invention have a diameter between 10 and 100 nm.

### Uses in Medicine of the Fusion Protein and the Nanoparticle of the Invention

In another aspect, the invention relates to a fusion protein or a nanoparticle according to the invention for use in medicine. In another aspect, the invention relates to the use of a fusion protein or a nanoparticle according to the invention for the treatment of a patient suffering from a disease that responds to the therapeutic agent forming part of the fusion protein of the invention.

As used herein, the terms “treat”, “treatment” and “treating” refer to the reduction or amelioration of the progression, severity and/or duration of a condition, disorder or disease, or the amelioration of one or more symptoms (preferably, one or more discernible symptoms) of a condition, disorder or disease. The terms “treat”, “treatment” and “treating” also refer to the amelioration of at least one measurable physical parameter of a condition, disorder or disease not necessarily discernible by the patient. Furthermore, “treat”, “treatment” and “treating” refer also to the inhibition of the progression of a condition, disorder or disease, either physically by, e.g., stabilization of a discernible symptom, physiologically by, e.g., stabilization of a physical parameter, or both. “Treat”, “treatment” and “treating” may refer, too, to the reduction or stabilization of the condition, disorder or disease.

It will be understood by the person skilled in the art that by use in medicine, the fusion protein or nanoparticle of the invention can be administered to a patient in order to induce a therapeutic response.

The therapeutic response comprises the suppression, reduction or arrest of the causes of the pathological condition or the disease suffered by a patient; the elimination, reduction, arrest or amelioration of the symptoms of the condition or disease; or the extinction, arrest or slowing down of the progression of the condition or disease in the patient.

The person skilled in the art will acknowledge that the fusion protein or nanoparticle of the invention suitable for use in medicine may be presented accompanied by a pharmaceutically acceptable carrier. As used herein, the term “pharmaceutically acceptable carrier” means a non-toxic, inert solid, semi-solid or liquid filler, diluent, encapsulating material or formulation auxiliary of any type. Remington's Pharmaceutical Sciences. Ed. by Gennaro, Mack Publishing, Easton, Pa., 1995 discloses various carriers used in formulating pharmaceutical compositions and known techniques for the preparation thereof.

Accordingly, the compositions comprising the fusion protein or nanoparticle of the invention and a pharmaceutically acceptable carrier are pharmaceutical compositions.

The pharmaceutical compositions of this invention can be administered to a patient by any means known in the art including oral and parenteral routes. According to such embodiments, inventive compositions may be administered by injection (e.g., intravenous, subcutaneous or intramuscular, intraperitoneal injection), rectally, vaginally, topically (as by powders, creams, ointments, or drops), or by inhalation (as by sprays)

### A—Use of the Fusion Protein or the Nanoparticle of the Invention in the Treatment of Cancer.

Another embodiment of the invention relates to a fusion protein and the nanoparticle of the invention, or their corresponding pharmaceutical compositions, wherein the polycationic peptide is a sequence capable of specifically interacting with a receptor on a cell surface which is capable of promoting the internalization of the fusion protein into the cell, wherein said cell expressing the receptor is a tumor cell present in cancer, and wherein the therapeutic agent is selected from the group consisting of
(i) A chemotherapy agent,
(ii) a cytotoxic polypeptide,
(iii) an antiangiogenic polypeptide,
(iv) a polypeptide encoded by a tumor suppressor gene,
(v) a pro-apoptotic polypeptide,
(vi) a polypeptide having anti-metastatic activity,
(vii) a polypeptide encoded by a polynucleotide which is capable of activating the immune response towards a tumor and
(viii) an antiangiogenic molecule.
(ix) a toxin
   for use in the treatment of cancer.

As used herein, the terms “treat”, “treatment” and “treating” refer to the reduction or amelioration of the progression, severity and/or duration of cancer, or the amelioration of one or more symptoms (preferably, one or more discernible symptoms) of cancer. The terms “treat”, “treatment” and “treating” also refer to the amelioration of at least one measurable physical parameter of cancer, such as growth of a tumor, not necessarily discernible by the patient. Furthermore, “treat”, “treatment” and “treating” refer also to the inhibition of the progression of cancer, either physically by, e.g., stabilization of a discernible symptom, physiologically by, e.g., stabilization of a physical parameter, or both. “Treat”, “treatment” and “treating” may refer, too, to the reduction or stabilization of tumor size or cancerous cell count.

The term “cancer” refers to a group of diseases involving abnormal, uncontrolled cell growth and proliferation (neoplasia) with the potential to invade or spread (metastasize) to other tissues, organs or, in general, distant parts of the organism; metastasis is one of the hallmarks of the malignancy of cancer and cancerous tumors. The abnormal growth and/or proliferation of cancerous cells is the result of a combination of genetic and environmental factors that alter their normal physiology. The growth and/or proliferation abnormalities of cancerous cells result in physiological disorders and, in many cases, death of the individual, due to the dysfunctionality or loss of functionality of the cell types, tissues and organs affected.

The term “cancer” includes, but is not restricted to, cancer of the breast, heart, small intestine, colon, spleen, kidney, bladder, head, neck, ovaries, prostate gland, brain, pancreas, skin, bone, bone marrow, blood, thymus, womb, testicles, hepatobiliary system and liver; in addition to tumors such as, but not limited to, adenoma, angiosarcoma, astrocytoma, epithelial carcinoma, germinoma, glioblastoma, glioma, hemangioendothelioma, hemangiosarcoma, hematoma, hepatoblastoma, leukemia, lymphoma, medulloblastoma, melanoma, neuroblastoma, hepatobiliary cancer, osteosarcoma, retinoblastoma, rhabdomyosarcoma, sarcoma and teratoma. Furthermore, this term includes acrolentiginous melanoma, actinic keratosis adenocarcinoma, adenoid cystic carcinoma, adenomas, adenosarcoma, adenosquamus carcinoma, astrocytic tumors, Bartholin gland carcinoma, basal cell carcinoma, bronchial gland carcinoma, capillary carcinoid, carcinoma, carcinosarcoma, cholangiocarcinoma, cystadenoma, endodermal sinus tumor, endometrial hyperplasia, endometrial stromal sarcoma, endometrioid adenocarcinoma, ependymal sarcoma, Ewing sarcoma, focal nodular hyperplasia, germ cell tumors, glioblastoma, glucagonoma, hemangioblastoma, hemagioendothelioma, hemagioma, hepatic adenoma, hepatic adenomastosis, hepatocellular carcinoma, hepatobilliary cancer, insulinoma, intraepithelial neoplasia, squamous cell intraepithelial neoplasia, invasive squamous-cell carcinoma, large cell carcinoma, leiomyosarcoma, melanoma, malignant melonoma, malignant mesothelial tumor, medulobastoma, medulloepithelioma, mucoepidermoid carcinoma, neuroblastoma, neuroepithelial adenocarcinoma, nodular melanoma, osteosarcoma, papillary serous adenocarcinoma, pituitary tumors, plasmacytoma, pseudosarcoma, pulmonary blastoma, renal cell carcinoma, retinoblastoma, rhabdomyosarcoma, sarcoma, serous carcinoma, microcytic carcinoma, soft tissue carcinoma, somatostatin secreting tumor, squamous carcinoma, squamous cell carcinoma, undifferentiated carcinoma, uveal melanoma, verrucous carcinoma, vipoma, Wilm tumor, intracerebral cancer, head and neck cancer, rectal cancer, astrocytoma, glioblastoma, microcytic cancer and non-microcytic cancer, metastatic melanoma, androgen-independent metastatic prostate cancer, androgen-dependent metastatic prostate cancer and breast cancer.

Thus, in a preferred embodiment of the invention, the therapeutic agent is selected from the group consisting of
(i) a cytotoxic polypeptide,
(ii) an antiangiogenic polypeptide,
(iii) a polypeptide encoded by a tumor suppressor gene,
(iv) a pro-apoptotic polypeptide,
(v) a polypeptide having anti-metastatic activity,
(vi) a polypeptide encoded by a polynucleotide which is capable of activating the immune response towards a tumor,
(vii) a chemotherapy agent and
(viii) an antiangiogenic molecule.

In a more preferred embodiment of the invention, the antitumor peptide of the fusion protein or the nanoparticle of the invention is selected from the group consisting of the BH3 domain of BAK, PUMA, GW-H1, and the active segment of diphtheria toxin I, and the Pseudomonas aeruginosa exotoxin A.

As used herein “BAK” refers to the well-known pro-apoptotic factor belonging to the Bcl-2 protein family that triggers programmed cell death by caspase-dependent apoptotic pathway through inactivating anti-apoptotic proteins, permeabilizing the mitochondrial membrane, and consequently, releasing cytochrome C and other mitochondrial cell death factors. [as seen in Llambi, F. et al. 2011. Mol. Cell, 44:517-31]. In one embodiment, BAK refers to full length BAK (SEQ ID NO: 41). In other embodiment, BAK refers to any truncated form thereof containing the functional BH3 domain (SEQ ID NO: 42).

As used herein, “PUMA” refers to a protein characterized by a full sequence corresponding to SEQ ID NO: 43) which is a (Bcl-2 homology 3) BH3-only protein that triggers cell death by interacting with pro and antiapoptotic proteins of the Bcl-2 family.

As used herein, GW-H1 refers to a polypeptide having the sequence of SEQ ID NO: 14 which exerts its cytolytic activity by folding into an amphipathic helix

Diphtheria toxin I (produced by the bacteria of the species Corynebacterium diphtheriae) (SEQ ID NO: 44) and the exotoxin of P. aeruginosa (SEQ ID NO: 45) belong to the family of ADP-ribosilating toxins. Both toxins are proteins that act on eukaryotic Elongation Factor-2 (eEF-2), basically inhibiting the translational activity of the cell that incorporates them and inducing apoptosis. The structure of both toxins presents a receptor-binding domain (that binds to a surface receptor of the cell and induces endocytosis; heparin binding epidermal growth factor precursor in the case of diphtheria toxin, CD91 in the case of the exotoxin A), a translocation domain, and a catalytic domain that performs the action on eEF-2 (an overview is provided in Shapira, A. & Benhar, I., 2010, Toxins, 2:2519-2583).

In an even more preferred embodiment of the invention, the polycationic peptide of the fusion protein or the nanoparticle of the invention is a CXCR4 ligand, and the cancer targeted to be treated with the fusion protein or the nanoparticle of the invention is characterized by comprising cells which express the CXCR4 receptor. In a more preferred embodiment, the cells cancer cells that express or overexpress CXCR4 are metastatic stem cells. The term “metastatic stem cells”, as used herein, refers to cells that are responsible for metastasis initiation and metastasis maintenance

In a yet more preferred embodiment of the invention, the CXCR4 ligand of the fusion protein or the nanoparticle of the invention is selected from the group comprising the T22 peptide, the V1 peptide, the CXCL12 peptide, the vCCL2 peptide or a functionally equivalent variant thereof.

In another more preferred embodiment of the invention, the cancer to be treated with the fusion protein or the nanoparticle of the invention is selected from the group consisting of pancreatic and colorectal cancer.

In another preferred embodiment of the invention, the fusion protein and the nanoparticle of the invention are used for the treatment of cancerous tumor, wherein the cancerous tumor is a primary tumor or a metastasis.

The invention will be described by way of the following examples which are to be considered as merely illustrative and not limitative of the scope of the invention.

### EXAMPLES

### Materials and Methods

### Synthesis of 5′-dimethoxytrityl-5-fluoro-2′-deoxyuridine

5-Fluoro-2′-deoxyuridine (FdU) (3.2 mmol) was reacted with dimethoxytrityl chloride (4.4 mmol) in dry pyridine. The solution was stirred overnight and the solvent was evaporated. The residue was purified by column chromatography using a gradient from pure CH2Cl2 to 10% methanol in CH2Cl2 to afford the desired dimethoxytrityl-5-fluoro-2′-deoxyuridine (DMT-FdU) in 65%.

### Synthesis of 5′-Dimethoxytrityl-5-fluoro-2′-deoxyuridine phosphoramidite

5′-Dimethoxytrityl-5-fluoro-2′-deoxyuridine (1 mmol) was dried by evaporation with anhydrous acetonitrile under reduced pressure. Next the product was dissolved in anhydrous CH₂Cl₂ (20 mL) under argon and diisopropylethylamine (5 mmol) was added with exclusion of moisture. The solution was cooled with an ice bath and 2-cyanoethoxy-N,N-diisopropylamino-chlorophosphine (2 mmol) was added dropwise with a syringe. The solution was stirred at room temperature for 1 h and, then, the solution was diluted with CH₂C₁₂ and washed with saturated aqueous NaCl. After drying the organic phase with anhydrous Na₂SO4, the solvent was evaporated and the product was purified by column chromatography. The column was packed with silica gel using a 10% tritehylamine solution in ethyl acetate/hexane 1:1 and the compound was eluted with a gradient from ethyl acetate/hexane 1:1 to pure ethyl acetate. The desired phosphoramidite was obtained in 70% yield.

Preparation of the Solid Support functionalized with 5′-dimethoxytrityl-5-fluoro-2′-deoxyuridine

The DMT-FdU derivative (0.4 mmol) was dried by evaporation with anhydrous acetonitrile and reacted with succinic anhydride (0.6 mmol) and N,N-dimethylaminopyridine (0.6 mmol) in CH2Cl2 (20 mL). The reaction mixture was stirred overnight at room temperature. The reaction mixture was diluted with 30 mL of CH₂Cl₂ and the solution was washed with saturated aqueous NaCl, 10% citric acid aqueous solution and again with saturated aqueous NaCl. The organic layer was dried over anhydrous Na₂SO₄ and evaporated to dryness. The resulting DMT-FdU hemisuccinate was obtained as a white solid (89% yield) and was used in the next step without further purification. The DMT-FdU hemisuccinate derivative was incorporated on a long-chain alkylamine-controlled pore glass support (LCAA-CPG). Amino-LCAA-CPG (CPG New Jersey; 450 mg, 70 μmol amino/g) was placed into a polypropylene syringe fitted with a polypropylene disc and washed sequentially with methanol, CH2Cl2 and acetonitrile. Then 2,2′-dithiobis(5-nitropyridine) (0.18 mmol) dissolved in 0.6 mL of a mixture of acetonitrile/CH₂Cl₂ (1:3) was added to a solution of DMT-FdU hemissuccinate (0.9 mmol) and N.N-dimethylaminopyridine (0.18 mmol) in acetonitrile (1.5 mL). Next, triphenylphosphine (0.18 mmol) was added and the mixture was stirred for a few seconds, added to the support and allowed to react for 1 h. The support was washed with methanol, CH₂Cl₂ and acetonitrile and dried under vacuum. The functionality of the resin was determined by DMT quantification (42 μmol/g). Finally, the solid support was treated with an acetic anhydride solution during 30 min to cap the unreacted amino groups.

### Synthesis of the Floxuridine Oligonucleotide (Oligo-FdU)

Two pentanucleotide sequences were prepared: 1) 5′-(FdU) 5-3′ is the control FdU pentamer; and 2) 3′-thiolated FdU pentamer: 5′-(FdU) 5-HEG-propyl-SH 3′ with FdU 5-fluoro-2′-deoxyuridine and HEG as hexaethyleneglycol spacer. To synthetize the control 5′-(FdU) 5-3′ pentamer, a controlled pore glass (CPG) support functionalized with DMT-FdU prepared as described above was used. Then, the control pentamer sequence was assembled on a DNA synthesizer (392 Applied Biosystems, Foster City, CA, USA) using a 1 μmol synthesis cycle by successive additions of DMT-protected FdU phosphoramidite. After assembling of the sequence, oligonucleotide support was treated with aqueous ammonia (32%) for 2 hrs at room temperature and the resulting product was purified by HPLC. HPLC conditions: column X-bridge™ OST C18 (10×50 mm, 2.5 μm); 20 min linear gradient from 0% to 40%, flow rate 2 mL/min; solution A was 5% acetonitrile in 0.1 M aqueous triethylammonium acetate (TEAA) and solution B 70% acetonitrile in 0.1 M aqueous TEAA. Pentamer was characterized by mass spectrometry (MALDI-TOF).

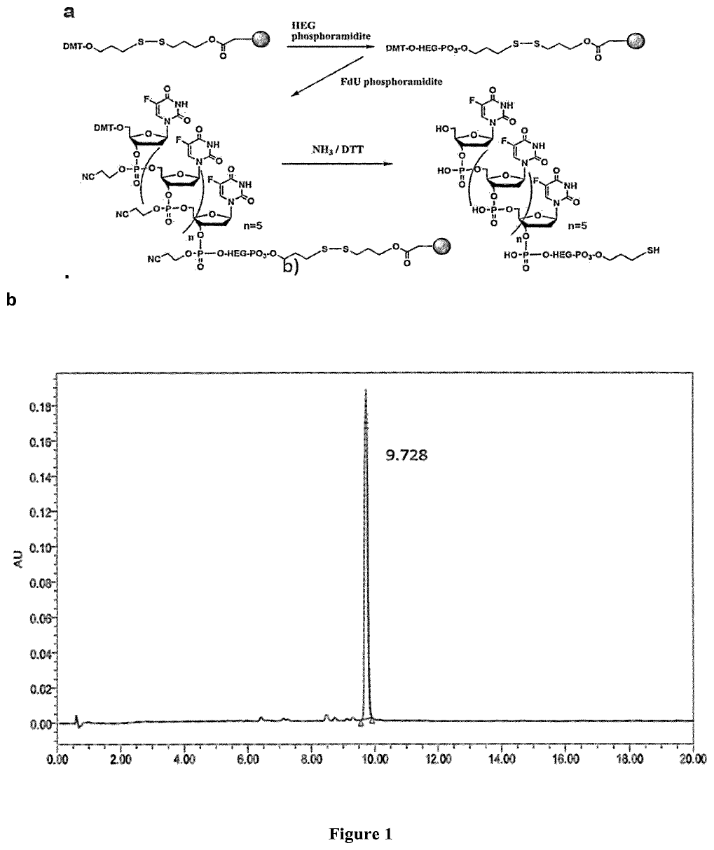

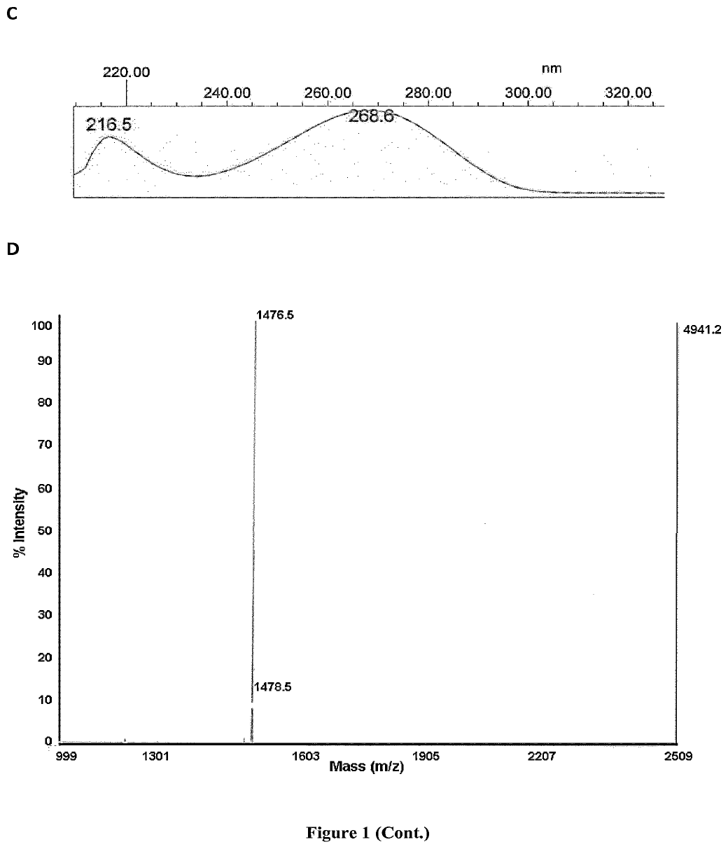

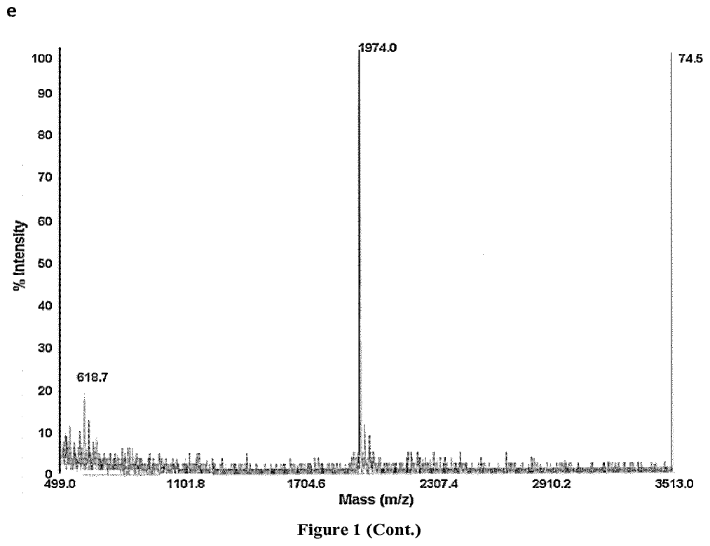

Several batches of pentamer FdU oligonucleotide were synthesized in 1 μmol scale on an automated RNA/DNA synthesizer using β-cyanoethylphosphoramidite chemistry and following standard protocols. 3′-Thiol-Modifier C3 solid support (Link Technologies) was used for the introduction of the thiol group at the 3′-end, then hexaethyleneglycol phosphoramidite (Glen Research, VA, USA) was used as spacer. Finally, the synthesis was completed by addition the repetitive additions of the DMT-protected FdU phosphoramidite. After the assembly of the sequence, oligonucleotide support was treated with aqueous ammonia (32%) with 0.1 M 1,4-dithiothreitol (DTT) for 2 h at room temperature. The ammonia solution was concentrated to dryness and the product was desalted on NAP-10 (Sephadex G-25) columns eluted with water prior to use. The purity of the pentamer FdU-HEG-SH was analyzed by HPLC using the conditions described above (see FIG. 1). Pentamer was quantified by absorption at 260 nm and confirmed by MALDI mass spectrometry (MALDI-TOF).

### Characterization of the T22-GFP-H6-FdU Therapeutic Nanoconjugate and Determination of Drug Nanoparticle Ratio

The products obtained after the T22-GFP-H6-FdU synthesis reaction were characterized using mass spectroscopy to measure their molecular mass. The volume and size distribution of the nanoparticles was determined by dynamic light scattering at 633 nm (Zetasizer Nano ZS, Malvern Instruments Limited, Malvern, Worcestershire, UK). Nanoconjugate size was also measured using purified samples, diluted to 0.2 mg/mL and contrasted by evaporation of 1 nm platinum layer in carbon-coated grids, before being visualized in a Hitachi H-7000 transmission electron microscope (TEM), as described 33. The drug to nanoparticle ratio was obtained by analyzing the UV spectra of T22-GFP-H6 and T22-GFP-H6-FdU nanoconjugate and calculating the number of FdU molecules per T22-GFP-H6 nanoparticle.

### T22-GFP-H6-FdU Internalization, CXCR4 Specificity and Cytotoxicity in CXCR4⁺ HeLa Cells

T22-GFP-H6-FdU capacity to internalize in a second cell type was assessed: the CXCR4⁺ HeLa human cervical carcinoma cell line, cultured in Minimum Essential Medium medium supplemented with 10% FBS and 2 mM Glutamax (Gibco), by exposing cells for 1 hour to 1 μM T22-GFP-H6-FdU concentration and measuring the green fluorescence emitted by the internalized cells in the flow cytometer FACS-Canto system (Becton Dickinson). To assess specificity for CXCR4 receptor mediated internalization, competition studies incubating CXCR4⁺ HeLa cells with the CXCR4 antagonist AMD3100 (octahydrochloride hydrate, Sigma-Aldrich) were performed in a 1:10 (Protein: antagonist) molar ratio for 1 h before exposure to the nanoconjugate, as previously described for the T22-GFP-H6.

The T22-GFP-H6-FdU subcellular localization was assessed using confocal microscopy Briefly: for confocal analysis, cells were grown on culture dishes (Mat-Tek Co.); then, T22-GFP-H6-FdU was added in OptiPro medium supplemented with L-Glutamine. Nuclei were labeled with Hoechst 33342 (Life Technologies) and plasma membranes CellMask™ Deep Red (Life Technologies). Micrographs were taken by TCS-SP5 confocal laser scanning microscopy (Leica Microsystems, Wetzlar, Germany) and 3D models of T22-GFP-H6-FdU localization were generated using Imaris software (Bitplane)

T22-GFP-H6-FdU cytotoxic activity was also studied by using the MTT metabolic test (Roche). To that purpose, CXCR4⁺ HeLa cells were exposed to T22-GFP-H6-FdU at 1-1,000 nM concentration range and their viability measured at 48 hours as compared to equimolecular concentrations or free oligo-FdU. Afterwards, a graphic displaying the linearized T22-GFP-H6-FdU dose-response trend line representation to compare cell viability for both compounds was constructed. Reduction of cell viability was also determined by optical microscope images of HeLa cells exposed to 1 μM T22-197 GFP-H6-FdU for 48 h, as compared to T22-GFP-H6 or free FdU.

### Generation of CCR Model and Protocol for the Treatment of Established Metastases

A CXCR4⁺ SW1417 orthotopic CCR model in Swiss nude mice was used to evaluate the possible inhibition of established metastasis. T22-GFP-H6-FdU administration was started two month after tumor cell implantation (when metastases were already present in the mice measured by bioluminescence images using IVIS spectrum). To this purpose, 32 Swiss nude mice were randomized into four groups (buffer, T22-GFP-H6-FdU, T22-GFP-H6 and free FdU pentamer (n=10/each group). Single i.v. doses for each compound (T22-GFP-H6-FdU: 20 μg, T22-GFP-H6: 20 μg, free oligo-FdU at equimolecular doses or Buffer) were administered every three days for a total of 10 doses. The experiment was finished when the first animal of the buffer-treated group was euthanized.

### Evaluation of Antimetastatic Effect

The same evaluation applied in the treatment protocol was applied for metastasis prevention. Briefly, at necropsy the number and size of visible metastasis in all organs were recorder per mouse, ex vivo the number of metastatic foci that emitted bioluminiscence in the target organs were counted using the IVIS® 200-Spectrum and the histopathological and immunohistochemical analyses to confirm location and number of metastases were performed.

### T22-GFP-116-FdU Biodistribution and Toxicity in Bone Marrow and Circulating Blood Cells

The T22-GFP-H6-FdU uptake measuring fluorescence emission, in bone marrow and circulating blood cells, was assessed 5 h after the administration of T22-GFP-H6-FdU at 10-100 μg doses. At necropsy, bone marrow was extracted and registered ex vivo in the IVIS® 200-Spectrum equipment. Mouse blood was collected by punction in the maxillary plexus. The erythrocytes, leucocytes and platelets were isolated by the Ficoll density gradient method using the standard protocol. Further, pellets were obtained by centrifugation of the isolated cell extracts at 600 g, 10 min and then recorded to measure fluorescence using IVIS® 200-Spectrum.

### Synthesis of the T22-GFP-116-FdU Therapeutic Nanoconjugate

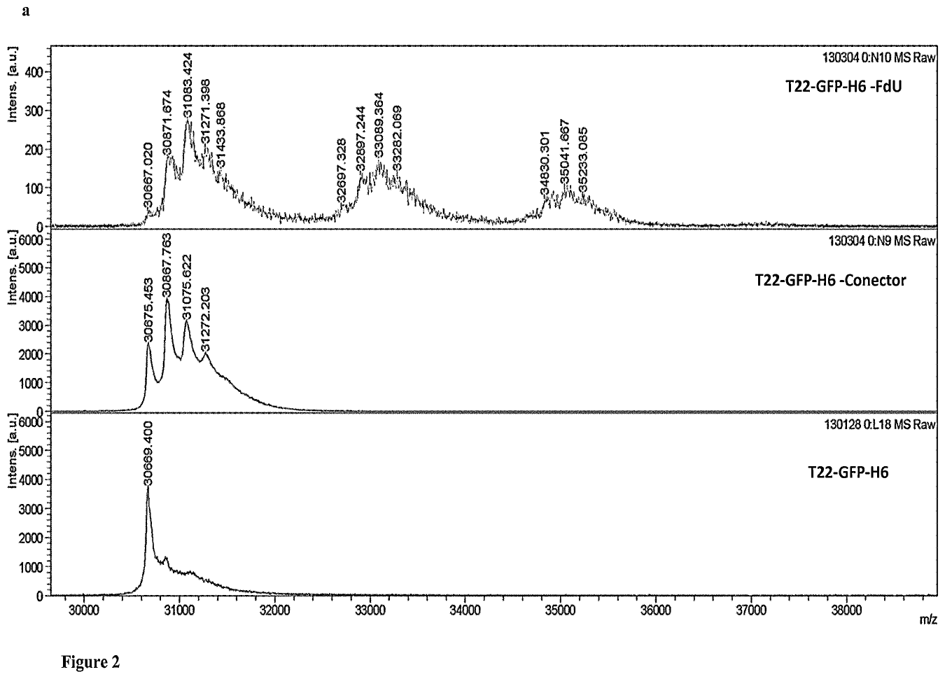

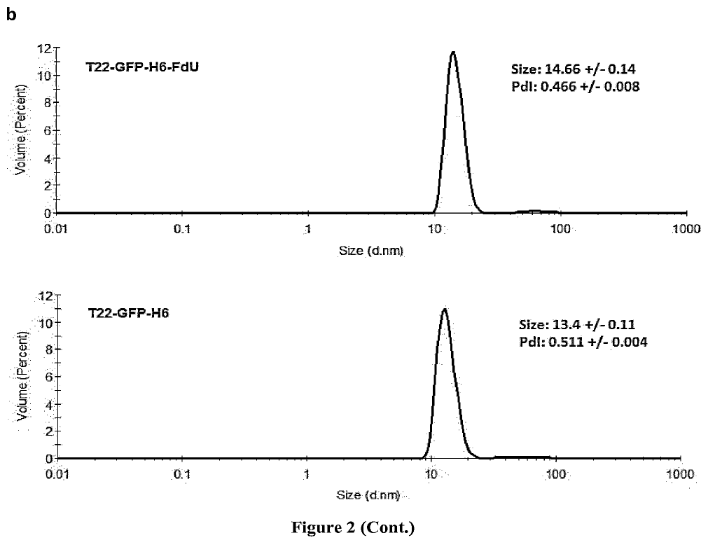

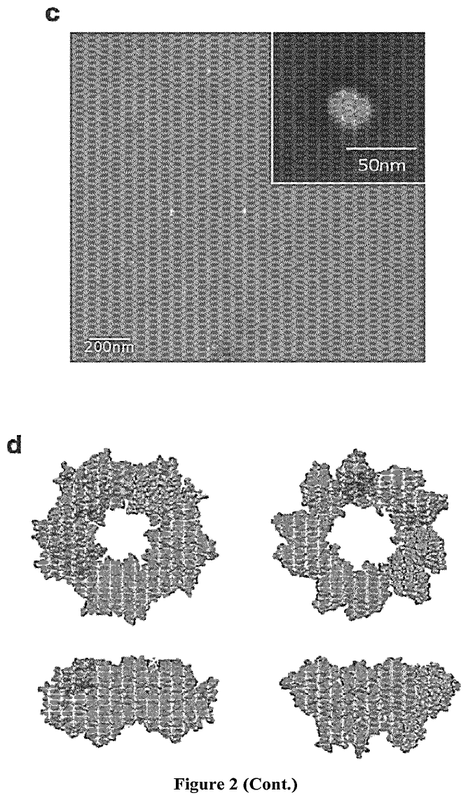

The nanoconjugate was synthesized by covalent binding of the targeting vector T22-GFP-H6, a protein nanoparticle produced in bacteria using a recombinant DNA strategy and oligo-FdU, a pentameric oligonuclotide of Floxuridine (5-Fluoro-2′-deoxyuridine) (Sigma Aldrich Chemie GmbH, Steinheim, Germany), both functionalized before their conjugation. The oligo-FdU was functionalized with sulfhydryl as described in FIG. 1. T22-GFP-H6 was functionalized by reacting with the linker MBHS (4-maleimido hexanoic acid N-hydroxysuccinimide ester) (Thermo Fisher), following the protocol for biofunctionalization of proteins described by Hermanson [Hermanson, G. 2013. Bioconjugate Techniques, 3rd Edition, ISBN9780123822390, Academic Press, London, pp. 1200]. This linker binds the amino groups of the external lysines of the T22-GFP-H6 protein adding maleimido groups. The final T22-GFP-H6-FdU nanoconjugate was obtained reacting T22-GFP-H6 functionalized with maleimide and oligo-FdU-thiol (Michael reaction). The final reaction product was purified by dialysis. The functionalization and physicochemical characterization of oligo-FdU with thiol is described in FIG. 1. The physicochemical and functional characterization of the reaction products for the synthesis of T22-GFP-H6-FdU is described in FIG. 2.

Determination of the ratio of oligo-FdU to T22-GFP-H6 in the resulting nanoconjugate was determined based on the absorbances of a 1 mg/ml solution of the T22-GFP-H6 conjugate and of a 1 mg/ml solution of the T22-GFP-H6-FdU conjugate at 260 nm of UV light. The difference in the absorbances between both solutions provides the absorbance of the FdU molecules present in the T22-GFP-H6-FdU solution. Taking into account Lamber Beer law (Abs-mg/ml*E*L wherein E-44500 M⁻¹·Cm⁻¹) and the molecular weight of the T22-GFP-H6 protein (MW=30,691 KDa), the number of moles of FdU per mg of T22-GFP-H6 protein were determined. Considering that the Absorbance of the T22-GFP-H6 is about 0.7/mg of protein, and the absorbance of the T22-GFP-H6-FdU is about 7.32/mg of fusion protein, results showed that 4.56 molecules of oligo-FdU are bound to each fusion protein.

### T22-GFP-16-FdU Internalization, CXCR4 Specificity and Cytotoxicity in CXCR4+ Cells In Vitro.

CXCR4+ SW1417-luc CRC cells were cultured in modified Eagle's medium (Gibco, Rockville, MD) supplemented with 10% fetal calf serum (Gibco), and incubated at 37° C. and 5% CO2 in a humidified atmosphere. The internalization capacity of the nanoconjugate was assessed by exposing CXCR4+SW141-luc cells (expressing the luciferase reporter gene) for 1 hour to 1 mM T22-GFP-H6-FdU concentration, treating them with 1 mg/mL trypsin (Gibco) for 15 minutes, and measuring the green emitted fluorescence of the internalized nanconjugate particles in the flow cytometer FACS Canto system (Becton Dickinson, Franklin Lakes, NJ), using a 15 mW air-cooled argon ion laser at 488 nm excitation. Fluorescence emission was measured with a D detector (530/30 nm band pass filter). To assess specificity for CXCR4 receptor-mediated internalization, competition studies were performed incubating CRC SW1417 cells with the CXCR4 antagonist AMD3100 (Sigma Aldrich) in a 1:10 (protein: antagonist) molar ratio for 1 h before exposure to the nanoconjugate at 1 mM for an additional hour.

To assess T22-GFP-H6-FdU subcellular localization, the cells were cultured in MatTek culture dishes (MatTek Corporation, MA, USA); then, T22-GFP-H6-FdU was added in OptiPro medium supplemented with L-Glutamine. The nuclei were labeled with 0.2 μg/mL Hoechst 33342 (Molecular Probes, Eugene, OR) and the plasma membranes with 2.5 μg/mL CellMask™ Deep Red (Molecular Probes) for 10 minutes in the dark. The cells were washed in phosphate-buffered saline (Sigma-Aldrich). Live cells were recorded by TCS-SP5 confocal laser scanning microscopy (Leica Microsystems, Heidelberg, Germany) using a Plan Apo 63×/1.4 (oil HC×PL APO lambda blue) objective as described. To determine particle localization inside the cell, stacks of 10-20 sections for every 0.5 μm of cell thickness were collected and three-dimensional models were generated using Imaris version 6.1.0 software (Bitplane, Zürich, Switzerland).

T22-GFP-H6-FdU cytotoxic activity was also studied by measuring cell viability and using the metabolic test 3-(4,5-dimethylthiazol-2-yl)-2,5-diphenyltetrazolium bromide (MTT, Roche, Basilea, Switzerland), following manufacturer recommendations. To that purpose, SW1417 CRC cells were exposed to T22-GFP-H6-FdU at 1.0-1,000 nM concentration range and measured their viability at 72 hours as compared to equimolecular concentrations of T22-GFP-H6 or free oligo-FdU. A dose-response curve was then constructed and the linearized T22-GFP-H6-FdU dose-response trend line for each compound was determined using the Sigmaplot vs. 10.0 software.

### Generation of CRC Mouse Models.

Three different CRC mouse models were used: one generated by subcutaneous CRC cell implantation to study nanoconjugate biodistribution and induction of CRC apoptosis and two generated by orthotopic cell implantation to study nanoconjugate biodistribution and antimetastaic effect, either for prevention of metastases or for induction of the regression of established metastases. To generate two of these models, five-week-old Swiss nude mice were used, whereas in one model NOD-SCID mice were employed. They were all female mice weighing 18 to 20 g (Charles River, L′Arbresle, France) and were housed in a sterile environment with bedding, water and y-ray-sterilized food ad libitum. Experiments were approved by the Mouse Ethics Committee at Hospital de la Santa Creu i Sant Pau.

### Subcutaneous (SC) Mouse CRC Model.

A subcutaneous CRC model was generated by injecting 1×107 CXCR4⁺ human SW1417-luci CRC cells (expressing luciferase, to allow bioluminescence follow-up of tumor growth) resuspended in 250 μl of media in the mouse flank. When tumors reached 700 mm3, they were excised and implanted SC tumor aliquots (3×3×3 mm) by the trocher system in a cohort of mice. SC model was used to assess tumor uptake and nanoconjugate internalization and in vivo competition studies using co-administration of the CXCR4 antagonist AMD3100. It was also used to determine the induction of DNA double strand breaks, tumor cell apoptosis and the fraction of CXCR4″ cancer cells remaining in tumor tissue (CXCR4⁺ CCF) along time after treatment, as described below. These data were then used to design the required dosage interval for the nanoconjugate repeated dose therapy in subsequent experiments aimed to determine its antimetastatic effect.

### Orthotopic (ORT) CRC Mouse Model Used to Study Regression of Established Metastases.

Swiss nude mice were anesthetized with ketamine and xylazine, exteriorizing their cecum by a laparotomy. 2×106 CXCR4⁺ SW1417-luci CRC cells (expressing luciferase, to allow ex vivo bioluminescent identification of metastatic foci in affected organs at the end of therapy) were suspended in 50 ml of modified Eagle's medium, and loaded into a sterile micropipette. The cell suspension was slowly injected, under a binocular lens, with an approximate 30° angle and its tip introduced 5 mm into the cecal wall. Afterward, a slight pressure was applied with a cotton stick at approximately 2 mm from the injection point in the direction of the pipette axis. The pipette was pulled out and the area around the injection cleaned with 3% iodine. After injection, the gut was returned to the abdominal cavity and closed with surgical staples. This model was used to evaluate the capacity of the T22-GFP-H6-FdU nanoconjugate to induce the regression of established metastases.

### Orthotopic (ORT) CRC Mouse Model to Study Prevention of Metastases.

A highly efficient metastastic model was generated in NOD/SCID mice, that received an intracecal microinjection (ORT) of SW1417-luci CRC cells disaggregated from SC tumours previously generated in a different cohort of NOD/SCID mice. Thus, a SC+ORT model, was produced: when SC tumors reached a volume of 700 mm3, mice were sacrificed by cervical dislocation and tumours were excised, discarding the necrotic areas. Three hundred mg of viable tumour tissue was then cut into pieces and disaggregated in a mix of 0.05% trypsin (Invitrogen) and 100 mg/ml DNase (Sigma-Aldrich). The mix was pipetted 30 times, using a 10 ml pipette, and incubated for 10 minutes at 37° C. with shaking. It was then re-pipetted 30 times, using 10 ml, 3 ml and 1 ml pipettes, and re-incubated for 5 minutes at 37° C. with shaking. This re-pipetting step was then repeated. The obtained SW1417 single-cell suspension was filtered through a cell strainer and centrifuged at 1,000 g for 10 minutes before counting the cells. Then 2×106 cells, previously grown in culture and resuspended in 50 μl of media, were microinjected in the cecum of each mouse, following the method described above. This model was used to evaluate the capacity of the T22-GFP-H6-FdU nanoconjugate to prevent metastasis development.

### T22-GFP-H6-FdU Tumor Uptake, Tumor Cell Internalization and Induction of DNA Damage and Apoptosis ‘In Vivo’.

The SC CXCR4⁺ SW1417 CRC model was used to assess the internalization of the T22-GFP-H6-FdU nanoconjugate into the cytosol of CXCR4⁺ tumor cells after the administration of 100 mg T22-GFP-H6-FdU as an i.v. single bolus compared with buffer, T22-GFP-H6 (untargeted nanoconjugate) and oligo-FdU (unconjugated free drug). Two hours, 5 h and 24 h after the administration, the mouse was euthanized to resect the tumor and register ex vivo the intensity of the green fluorescence emitted by the nanoconjugated that biodistributed to tumor tissue using the IVIS® 200-Spectrum (Perkin Elmer, MA, USA). Following, tumor tissue samples were taken, processed and performed immunohistochemistry (IHC) to determine the presence or absence of the corresponding nanoconjugate into cytosol of tumor cells using an anti-GFP antibody (1:300; Santa-Cruz Biotechnology, CA, USA). Double strand breaks (DSBs) formation was also measured by IHC using an anti-gH2AX mAb (1:400, Novus Biologicals, Cambridge, UK) and counted the number of stained cells in five magnification fields of different tumor sections in each mouse. Finally, the capacity of T22-GFP-H6-FdU for apoptosis induction was compared 24 h after the administration of an equimolecular dose of oligo-FdU or Buffer. Apoptosis was assessed determining nuclear condensation with Hoechst staining of tumor tissue sections and counting the number of condensed or defragmented nuclei in five high-power magnification fields in different sections for each tumor using the Olympus DP73 digital camera and the CellD Imaging 3.3 programme (Olympus, Tokyo, Japan).

Definition of the Optimal Dose Interval by Changes in CXCR4⁺ Tumor Cell Number after T22-GFP-H6-FdU Administration.

The SC SW1417 mouse model was used to determine the capacity of the nanoconjugate to induce DNA damage and apoptosis in tumor tissue, as well as the kinetics of CXCR4 expression in the membrane of tumor cells after treatment, regarding the fraction of CXCR4 expressing tumor cells and their intensity, since CXCR4⁺ are the target cells for the nanoconjugate. To that purpose 24, 48, 72 h after the administration of a single i.v. bolus of 100 μg T22-GFP-H6-FdU, the mice were euthanized and tumor samples taken, which later were fixed and paraffin-embedded to determine the levels and the percent of tumor cells expressing CXCR4 using IHC with an anti-CXCR4 antibody (1:300, Abcam, UK). The mice had been treated with equimolecular dosages of T22-GFP-H6, free oligo-FdU or Buffer, in which also were determined the levels of CXCR4 expression in tumor tissue at the different time points. The results of the kinetics of CXCR4 expression in tumor cells were used to establish the optimal T22-GFP-H6-FdU nanoconjugate administration interval in the repeated dose schedule used to evaluate antimetastatic effect.

### Treatment Protocol for the Evaluation of T22-GFP-H6-FdU Induction of Metastasis Regression.

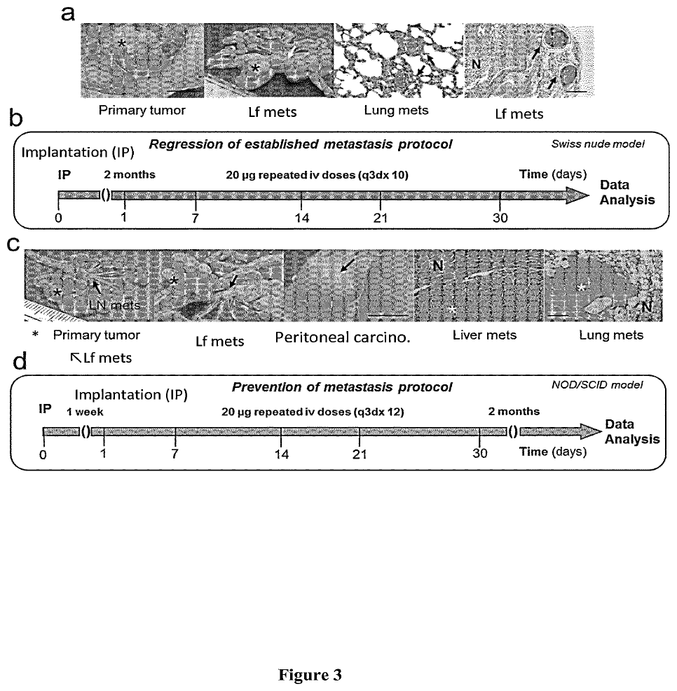

The orthotopic and metastatic CRC model developed in Swiss nude mice was employed to perform experiments of metastasis regression. 40 mice were randomized into four groups: buffer, T22-GFP-H6, T22-GFP-H6-FdU and free oligo-FdU (n=10/group) and administered repeated i.v. boluses at equimolecular doses, as follows: T22-GFP-H6-FdU 20 μg, free oligo-FdU: 2.6 nmols, or buffer), every three days (q3d) for a total of 10 doses. The T22-GFP-H6-FdU administration was initiated two months after tumor cell implantation, the time at which it was determined, in previous experiments, that lymph node and lung metastases were present (FIG. 3). The experiment was finished when the first animal of the buffer-treated-group had to be euthanized. See below the studied parameters to evaluate the antimetastatic effect.

### Treatment Protocol for the Evaluation of T22-GFP-H6-FdU Metastasis Prevention Effect.

The SC+ORT metastatic CRC model developed in NOD/SCID mice was used to evaluate the capacity of the nanoconjugate for metastasis prevention. 34 mice were randomly assigned into three groups: buffer (n=11), T22-GFP-H6-FdU (n=12) and free oligo-FdU (n=11) and administered repeated i.v. boluses at equimolecular doses, as follows: T22-GFP-H6-FdU 20 μg, free oligo-FdU: 2.6 nmols, or buffer), every three days (q3d) for a total of 12 doses. The T22-GFP-H6-FdU administration was initiated one week after tumor cell implantation before metastatic dissemination has occurred (FIG. 3). The experiment was finished when the first animal of the buffer-treated-group had to be euthanized.

### Evaluation of Antimetastatic Effect and Determination of the CXCR4+ Cancer Cell Fraction in Tumor Tissue at the End of Therapy.

At the end of both the regression and the prevention of metastasis experiments, the same methodology was applied to determine T22-GFP-H6-FdU antimetastatic effect. At necropsy, the number and size of visible metastases in the organs where dissemination is expected in colorectal cancer (lymph nodes, liver, lung and peritoneum) were recorded for each mouse in all compared groups. Also the number of metastatic foci that emitted bioluminiscence (derived from the SW1417-luci CRC cells) were counted ex vivo in the target organs for metastasis, using the IVIS® 200-Spectrum (Perkin-Elmer). Samples for histopathological and immunohistochemical analyses were collected and processed. Two independent observers analyzed H&E stained samples to count the number and measure the size of all observed metastatic foci in three (metastasis prevention experiment) or six (regression of metastasis experiment) sections of each organ in each mouse. The images were taken and the measurements were performed with an Olympus microscope with the CellD Olympus software (v3.3). CXCR4 expression in tumor tissue was determined using IHC with an anti-CXCR4 antibody, as described above, to determine the fraction of CXCR4⁺ cancer cells remaining in tumor tissue (CXCR4⁺ CCF) after treatment, including primary tumor and metastatic foci at the different organs affected by metastases (peritoneum, liver, lung and lymph nodes). The obtained results were used to study a possible correlation between CXCR4⁺ CCF and antimetastatic effect at the different sites.

### T22-GFP-H6-FdU Biodistribution and Toxicity in Normal Organs.

The T22-GFP-H6-FdU uptake was assessed by measuring the green fluorescence emitted by the GFP domain of the nanoconjugate, as well as DNA DSBs and apoptotic induction in normal (non-tumor) tissues using the methodology described above. In addition, two independent observers evaluated the possible histopathological alterations observed in H&E stained non-tumor tissue samples, searching for signs of toxicity. These tissues included CXCR4-expressing organs (despite expressing this receptor to a significantly lower level than in tumor tissue) where the nanoconjugate could accumulate such as the bone marrow and spleen and the toxicity in non-CXCR4 expressing organs, especially those in which the unconjugated oligo-FdU such as the liver, was also assessed.

### Statistical Analysis.

In order to analyze differences between control and experimental groups of affected mice in metastatic rates at the different organs, Fisher's exact test was used. The Mann-Whitney test was used to compare number and size of metastatic foci in the affected organs among groups. All quantitative values were expressed as mean±SE and the statistical tests were performed using SPSS version 11.0 (IBM, New York, USA). Differences among groups was considered significant at a p<0.05.

### Example 1: Physicochemical Characterization of HS-Oligo-FdU

The functionalized pentamer FdU-HEG-SH was quantified by absorption at 260 nm and confirmed by MALDI mass spectrometry (MALDI-TOF), yielding a MW of 1976.2, being the expected MW 1974.0. The control pentanucleotide (free oligo-FdU) characterized by mass spectrometry (MALDI-TOF) yield a MW of 1476.5, being the expected MW: 1478.1.

### Example 2: Physicochemical Characterization of the T22-GFP-H6-FdU Nanoconjugate and Determination of the Drug to Nanoparticle Ratio

The analysis of the conjugation products was performed by MALDI-TOFF spectra identifying the peaks corresponding to one or two molecules of pentaoligonucleotides of FdU bound to the nanoparticle with the MW indicated in FIG. 2. The T22-GFP-H6-FdU size was determined by dynamic light scattering, being 14.6+0.14, as compared to 13.4+0.11 for the control T22-GFP-H6 nanoparticle, a size consistent with that determined by transmission electron microscopy. The molecular weight of the T22-GFP-H6 nanoparticles, determined by SEC-MALS is of 477 kDa. Considering that the molecular weight of the T22-GFP-H6 polypeptide is of 30,691 kDa, this results in that each nanoparticle has approximately 15 monomers. A drug/nanoparticle ratio of 60 was obtained for the product obtained in the T22-GFP-H6-FdU nanoconjugate synthesis reaction based on the UV spectra of T22-GFP-H6 and T22-GFP-H6-FdU nano-conjugate. This product incorporates an average of 4 molecules of the pentamer oligo-FdU which, when assembled into a nanoparticle containing 15 fusion proteins monomers, correspond to a total of 60 FdU molecules, per T22-GFP-H6 nanoparticle.

### Example 3: Development of T22-GFP-H6-FdU, a Nanoconjugate that Targets CXCR4⁺ CRC Cells

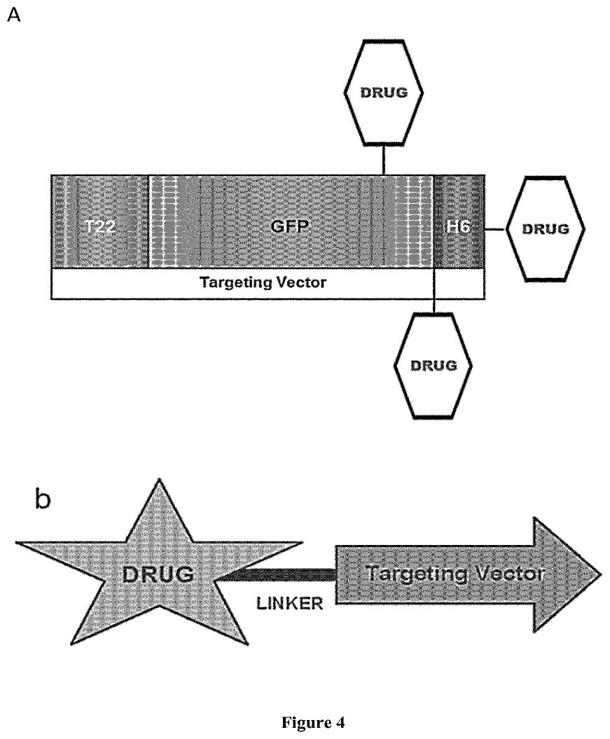

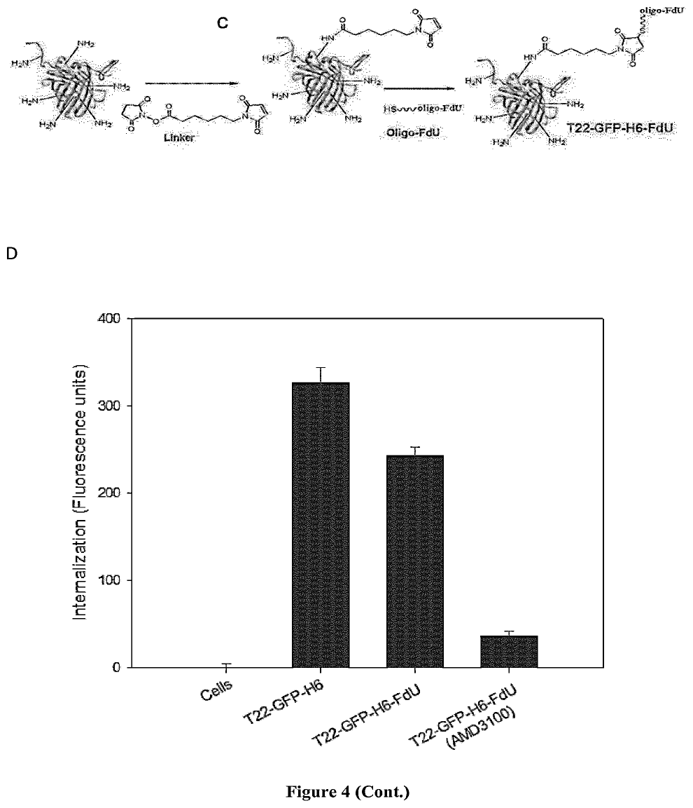

It has been previously demonstrated that CXCR4 overexpressing (CXCR4⁺) colorectal cancer (CRC) cells have metastasis initiation capacity (MIC) [Croker, A. K. & Allan, A. L. 2008. J Cell Mol Med 12, 374-390; Zhang, S. S. et al. 2012. BMC Med 10, 85 (2012)], and its inhibition by CXCR4 downregulation [Murakami, T. et al. 2012. BMC Med 10, 85; Wang, T. B. et al. 2014. Int J Oncol 44, 1861-1869] identifies these cells as metastatic stem cells (MetSCs). On this basis, the inventors generated a CXCR4-targeted nanoconjugate to evaluate if it could achieve antimetastatic effect by selectively eliminating CXCR4⁺ CRC cells. The structure and physicochemical characterization of this new T22-GFP-H6-FdU nanoconjugate is described in FIGS. 1, 2 and 4a-c. The conjugate contains T22 (a ligand that targets the CXCR4 receptor), a green fluorescent protein (allowing its in vivo follow-up) and oligo-FdU, an oligonucleotide of a drug active against CRC31, which allows to load a high number of drug molecules into the nanoconjugate. T22-GFP-H6-FdU was synthesized by functionalizing oligo-FdU with thiol (FIG. 4c and FIG. 1a), which was subsequently conjugated to a T22-GFP-H6 protein nanoparticle once bound to a chemical coupler (FIG. 4c). The inventors physico-chemically characterized the HS-oligo-FdU by HPLC, UV spectrometry and MALDI-TOF (FIG. 1b-e) and the final T22-GFP-H6-FdU product, by MALDI-TOF, dynamic light scattering (DIS) and Field Emission Scanning Electron Microscopy (FESEM) (FIG. 2a-c). This product had an approximate drug/nanoparticle (DNR) ratio of 40 (FIG. 2), a size slightly higher (14.66+0.14 nm) than T22-GFP-H6 and maintained its capacity for self-assembling (FIG. 2d). Moreover, its size was higher than the renal filtration cut-off (6-7 nm) ensuring a high recirculation time in blood, a requirement for effective targeted drug delivery.

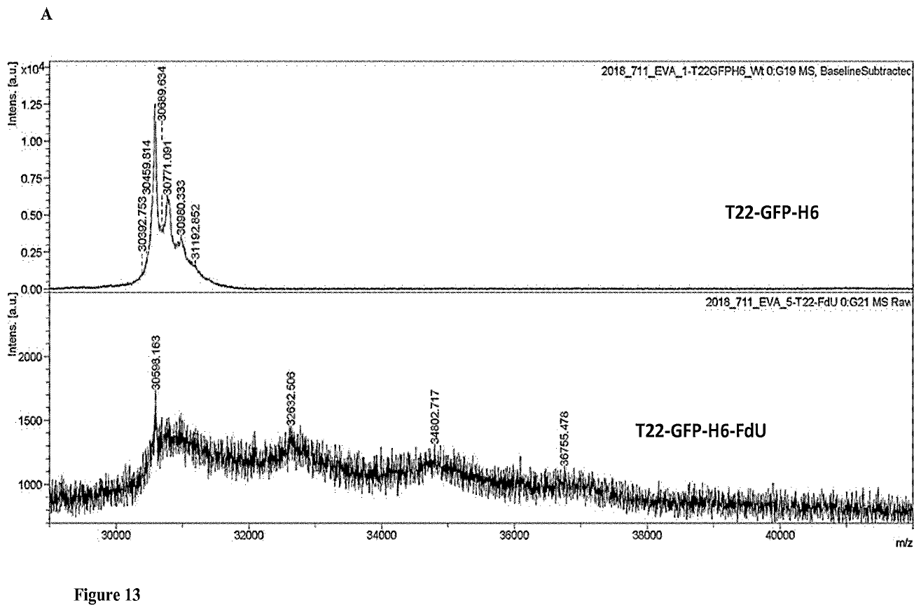

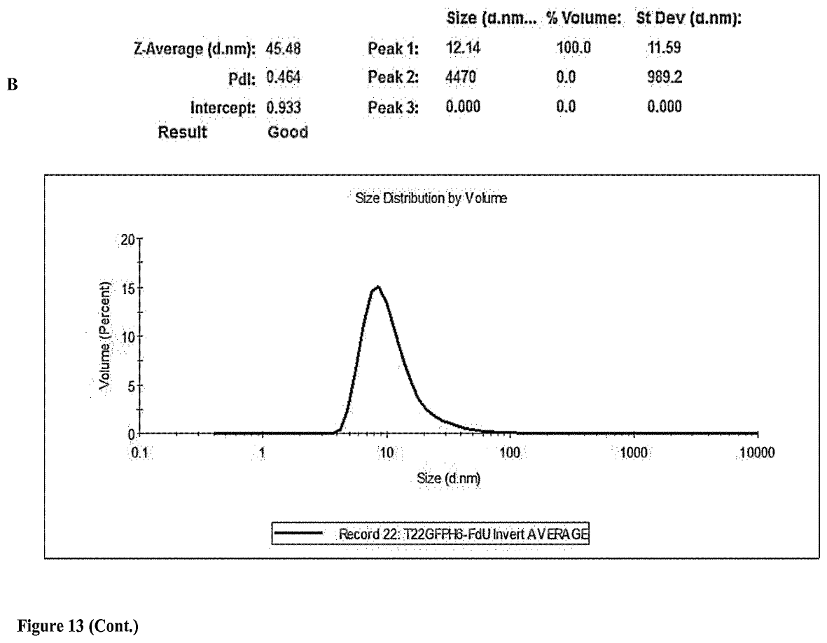

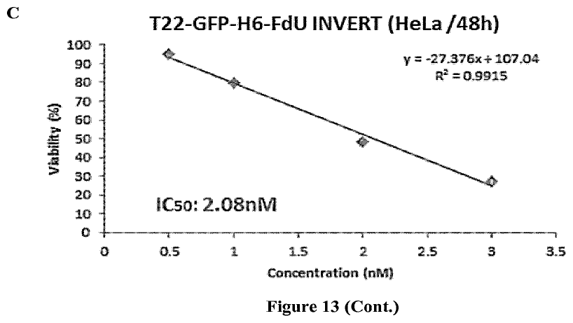

An inverted method was also used to produce T22-GFP-H6-FdU in which T22-GFP-H6 was first functionalized with a chemical coupler and then contacted with thiol-functionalized oligo-FdU. The conjugate obtained using this method (known as T22-GFP-H6-FdU INVERT) was then physico-chemically characterized by MALDI-TOF and dynamic light scattering (DLS) (FIGS. 13A and B).

### Example 4: T22-GFP-H6-FdU Selectively Internalizes and Kills CXCR4⁺ CRC Cells in Vitro

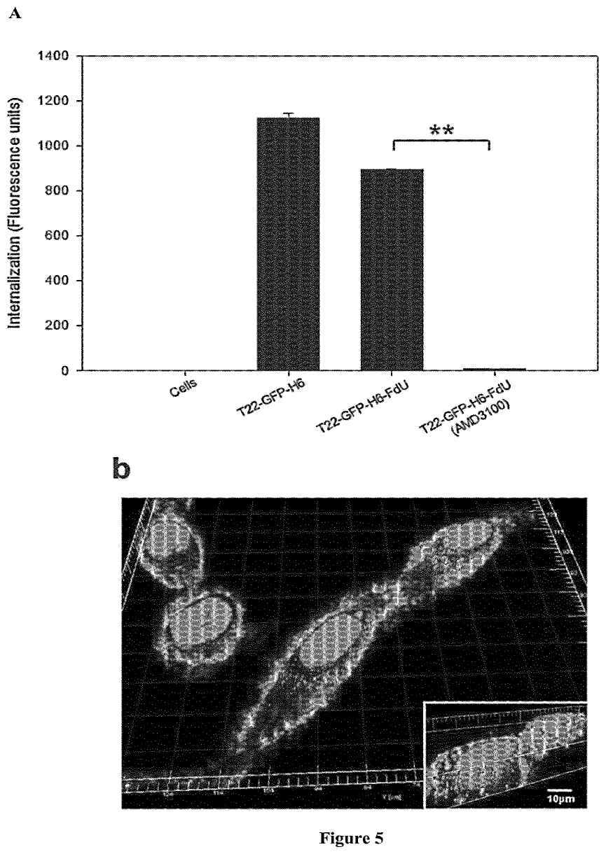

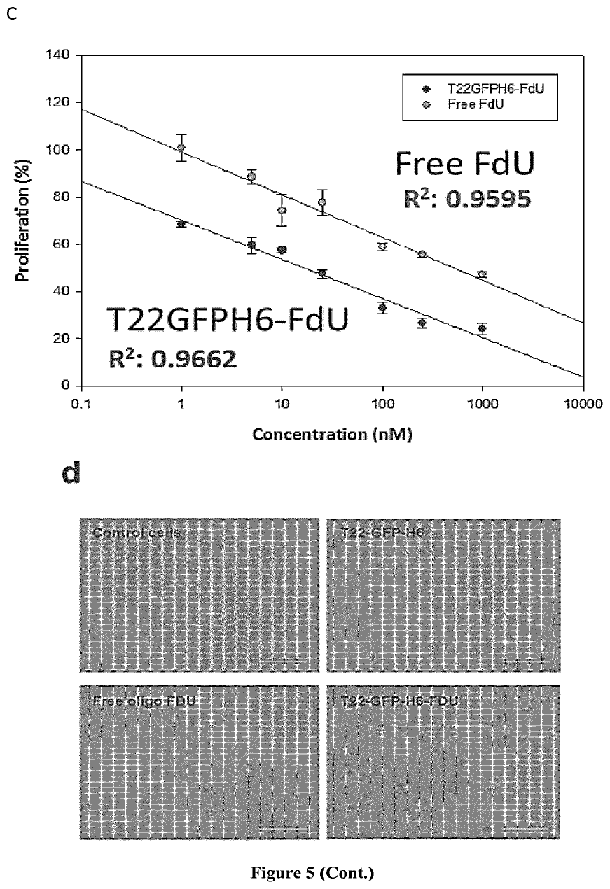

Following, the inventors assessed if the loaded oligo-FdU conferred the nanoparticle cytotoxic activity while maintaining its CXCR4 targeting capacity, provided that drug conjugation can alter protein conformation and function. T22-GFP-H6-FdU internalized in human CXCR4⁺ SW1417 CRC cells, as measured by fluorescence emission using flow cytometry, and accumulates and traffics in its cytosol, as observed by confocal microscopy (FIGS. 4d and 5a). The nanoconjugate maintains also its dependence on CXCR4 for internalization, since the AMD3100 CXCR4 antagonist was able to downregulate CXCR4 receptor in the membrane and completely blocked nanoconjugate internalization (FIG. 1d). In addition, T22-GFP-H6-FdU induced significantly higher cytotoxicity than free oligo-FdU in the same cells, as measured by cell viability or phase-contrast microscopy (FIG. 1g). The inventors confirmed CXCR4-dependent nanoconjugate internalization and higher cytotoxicity than free oligo-FdU in human CXCR4⁺ HeLa cells (FIG. 5a-d)

The T22-GFP-H6-FdU INVERT conjugate obtained using the inverted method was also characterized in terms of its cytotoxic effect on of CXCR4+ Hela cells (FIG. 13C).

### Example 5: T22-GFP-H6-FdU Selectively Targets CXCR4⁺ CRC Cells In Vivo

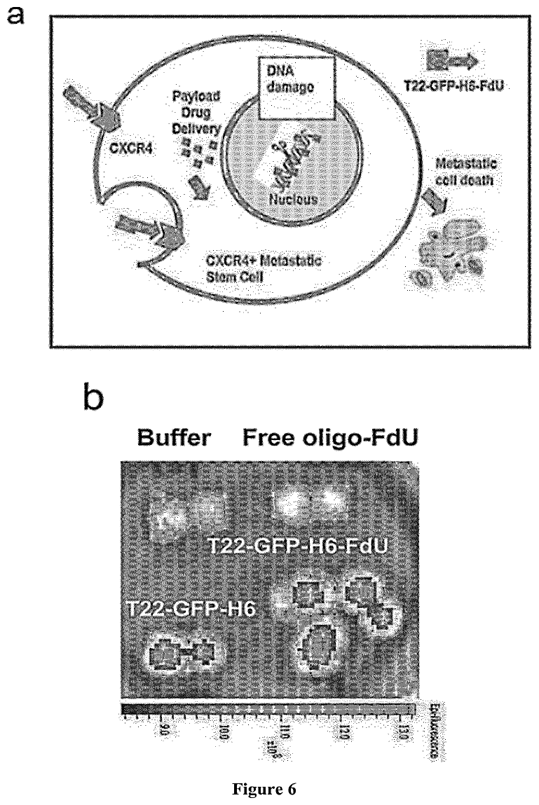

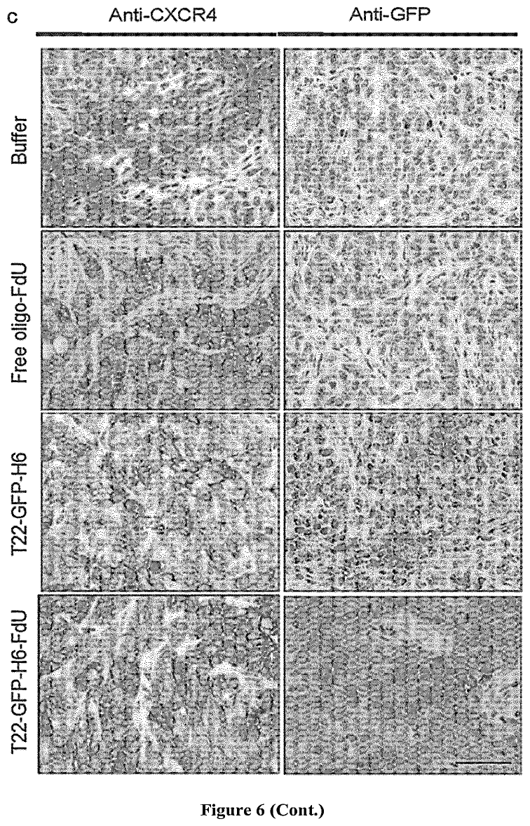

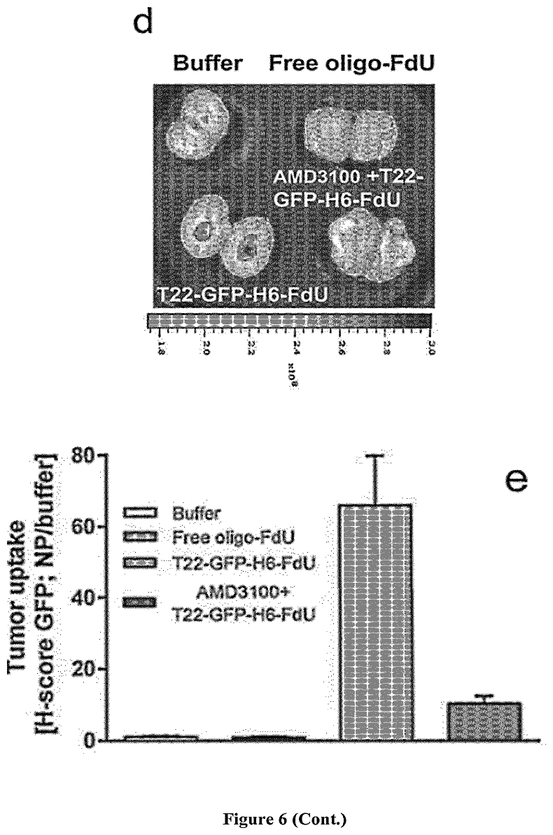

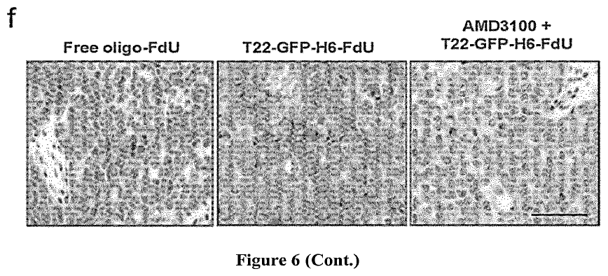

Once CXCR4-dependence for T22-GFP-H6-FdU in vitro activity was established, the inventors investigated in a subcutaneous (SC) CXCR4⁺ SW1417 CRC model if the nanoconjugate could achieve targeted drug delivery, after its intravenous administration, by assessing its selectivity and CXCR4-dependence regarding tumor tissue uptake, internalization in CXCR4 overexpressing MetSCs (target cells), intracellular release of the cytotoxic drug FdU and selective CXCR4⁺ MetSC killing (FIG. 6a). T22-GFP-H6-FdU showed selective tumor uptake, as measured by fluorescence emission, 5 h after the injection of a 100 mg dose in mice (FIG. 6b), as demonstrated for T22-GFP-H6 [Céspedes, M. V. et al. 2016. Nanomedicine. 12, 1987-1996.]. Moreover, both T22-GFP-H6-FdU and T22-GFP-H6 internalized into CXCR4⁺ tumor cell cytosol as measured by anti-GFP immunohistochemistry (IHC) (FIG. 6c), a signal undetectable in buffer or free oligo-FdU negative controls. In addition, administering to mice the CXCR4 antagonist AMD3100 prior to the nanoconjugate completely blocked its tumor uptake (FIG. 6d) as well as its internalization in CXCR4⁺ cancer cells (FIG. 6e-f). Therefore, the nanoconjugate achieves not only selective tumor biodistribution, but also its specific internalization into the cytosol of target CXCR4⁺ cancer cells, in a CXCR4-dependent manner.

### Example 6: T22-GFP-H6-FdU Achieves Targeted Drug Delivery Leading to Selective Depletion of CXCR4⁺ CRC Cells in CRC Tumors

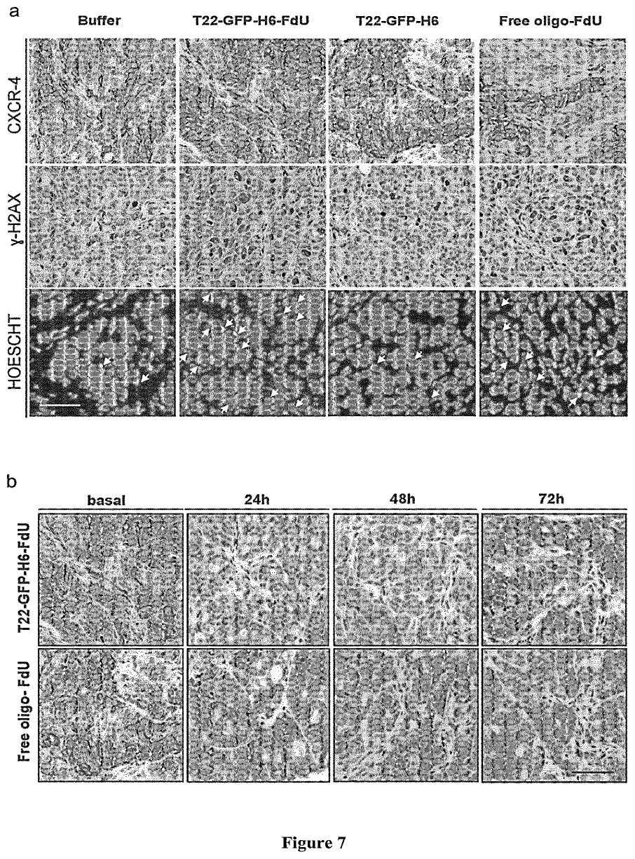

Next the same SC CRC model was used to assess if the selective internalization into the cytosol of CXCR4 target cancer cells achieved by the nanoconjugate leads to selective FdU delivery. The inventors also evaluated if the delivered FdU could induce DNA damage and apoptosis, triggering the specific elimination of CXCR4⁺ tumor cells, and if these effects differed from those achieved by free oligo-FdU. To that aim, H2AXg IHC was used to measure the generation of DNA double strand breaks (DSBs), since DSBs mediate FdU antitumor activity38. Five hours after T22-GFP-H6-FdU treatment the number of DSBs foci in tumors (22.8±1.4) was significantly higher p=0.02) than after free oligo-FdU (13.4±0.7), whereas DSBs were undetectable in T22-GFP-H6 or buffer-treated mice (FIG. 7a). T22-GFP-H6-FdU induction of DSBs indicated its capacity to release FdU in target cells to reach the nucleus and incorporate into DNA to induce DNA damage. Moreover, increased DSBs led to higher antitumor activity, since the number of apoptotic figures in tumor tissue, as measured by Hoechst nuclear condensation or defragmentation, 24 h after T22-GFP-H6-FdU injection (13.9±0.5) was significantly higher (p<0.05) than after free oligo-FdU (7.1±0.6), T22-GFP-H6 (3.0±0.3) or buffer (1.9±0.4) treatment (FIG. 7a).

Following, the fraction of CXCR4⁺ cancer cells (CXCR4⁺ CCF) remaining in tumor tissue was measured along time after a single 100 mg T22-GFP-H6-FdU dose, as compared to free oligo-FdU, using the SC CXCR4⁺ SW1417 CRC model. Before treatment, both groups showed a similar CXCR4⁺ CCF in tumor tissue (FIG. 7b); however, after T22-GFP-H6-FdU treatment the CXCR4⁺ CCF was reduced at 24 h and become undetectable at 48 h (FIG. 7b). Thus, 48 h post-administration T22-GFP-H6-FdU achieved selective elimination of CXCR4⁺ tumor cells in vivo. In contrast, the CXCR4+ CCF in tumor tissue after an equimolecular dose of free oligo-FdU remained similar to its basal level along time. Taken together, these results indicate that T22-GFP-H6-FdU achieves selective biodistribution to tumor tissue and FdU delivery to target CXCR4⁺ cancer cells, as indicated by an enhancement in DNA damage and tumor cell death, associated with selective elimination of CXCR4⁺ cancer cells, achieving, therefore, targeted FdU delivery to target cancer cells.

### Example 7: Transient Target Cell Elimination and Definition of a Dose Interval for Repeated T22-GFP-H6-FdU Injection

Despite T22-GFP-H6-FdU achieving selective depletion of CXCR4⁺ target cells in tumor tissue 48 h after its administration, this effect was found to be transient, since 72 h post-injection CXCR4⁺ cancer cell fraction in tumor tissue became similar to its level before therapy (FIG. 7b). In contrast, the CXCR4⁺ CCF in tumor tissue after free oligo-FdU therapy was maintained along time, remaining the same 24 h, 48 h or 72 h after treatment as before therapy (FIG. 7b). Therefore, in contrast to T22-GFP-H6-FdU effect, cancer killing by free oligo-FdU did not show selectivity towards CXCR4⁺ cancer cells. Based on these results, and in order to evaluate T22-GFP-H6-FdU antimetastatic effect, the inventors defined as optimal a 72 h (3 days) dose interval for its administration in a repeated dose schedule. It was expected that this schedule would maintain sufficiently low the fraction of CXCR4⁺ cancer cells (CXCR4⁺ CCF) remaining in primary tumors and metastatic foci, along the treatment period, so as to efficiently block metastatic dissemination and/or foci growth, provided that CXCR4⁺ cancer cells act as MetSCs. The T22-GFP-H6-FdU capacity to inhibit growth of established metastases was assessed by comparison to equimolecular doses of T22-GFP-H6 or free oligo-FdU by using an orthotopic bioluminescent CXCR4⁺ CRC model in Swiss nude mice, which generates Lymph node (LN) and lung metastases (FIG. 3a), and starting therapy 2 month after CRC cell implantation, at a 20 mg i.v. q3d dosage (FIG. 3b). The capacity of T22-GFP-H6-FdU to prevent metastasis, as compared to free oligo-FdU, was also assessed by using an orthotopic luminescent CXCR4⁺ CRC NOD/SCID model, with improved metastatic efficiency and dissemination at the LN, liver, lung and peritoneum (FIG. 3c), which started dosing one week after CRC implantation (FIG. 3d).

### Example 8: T22-GFP-H6-FdU Prevents Metastasis Development and Induces the Regression of Established Metastases

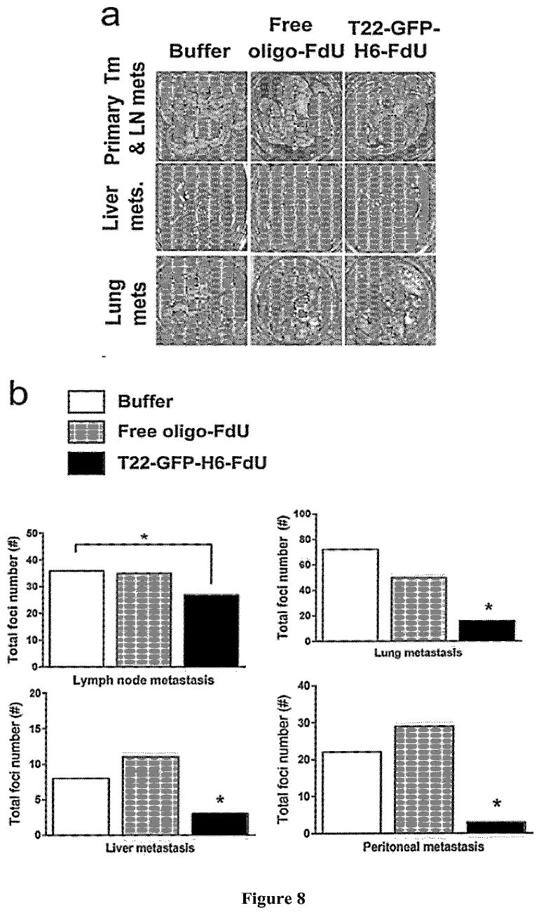

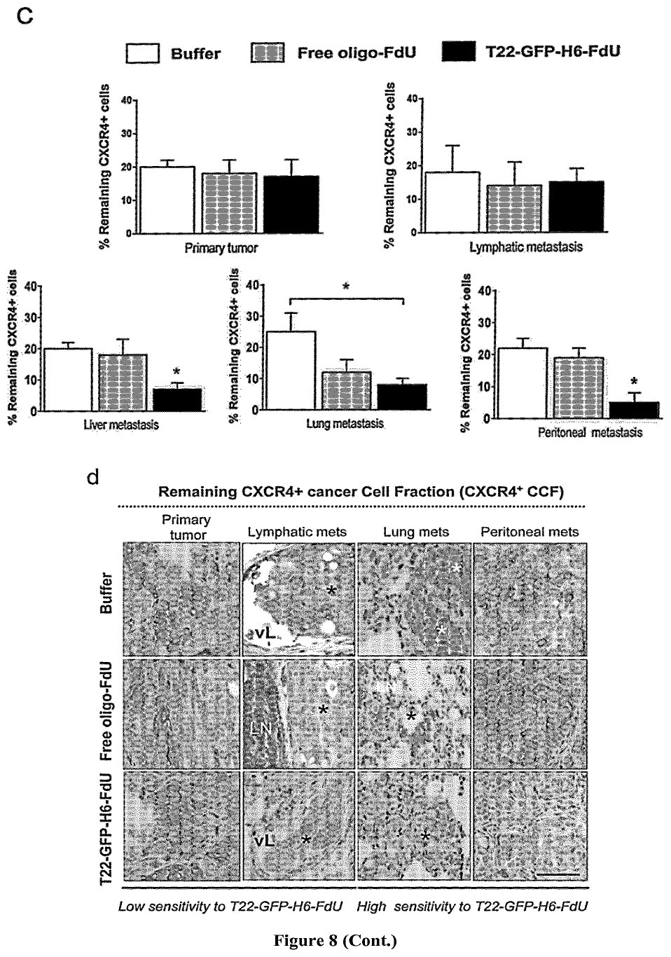

At the end of the prevention of metastasis experiment, and in contrast to free oligo-FdU effect, T22-GFP-H6-FdU blocked metastasis development in liver and lung, as measured by ex vivo luminescence (FIG. 8a). At the end of the experiment, a histological analysis showed that T22-GFP-H6-FdU-treated mice had a 10.3 fold reduction in the total number of metastases in the peritoneum (p=0.0001) and 3.1-3.7 times reduction in liver (p=0.001) or lung (p=0.006) than free oligo-FdU mice, whereas it did not prevent LN metastases (FIG. 8b). Similar results were obtained when comparing the mean foci number at the peritoneum, liver or lung per mouse between both groups (Table 1).

TABLE 1
T22-GFP-H6-FdU antimetastatic effect in the prevention and regression
of metastases experiments, measured as reduction in mean foci number
per mouse
Prevention of Metastasis Protocol

| Groups | LN foci | Liver foci | Lung foci | Peritoneal foci |
|---|---|---|---|---|
| Buffer | 3.7 ± 0.3 ^{a} | 0.7 ± 0.3 ^{b} | 6.6 ± 1.5 ^{d} | 2.0 ± 0.6 ^{f} |
| Free oligo-FdU | 3.1 ± 0.4 | 1.0 ± 0.3 ^{c} | 4.5 ± 1.6 ^{e} | 2.8 ± 1.0 ^{g} |
| T22-GFP-H6-FdU | 2.0 ± 0.4 ^{a} | 0.2 ± 0.1 ^{b, c} | 1.3 ± 0.9 ^{d, e} | 0.4 ± 0.3 ^{f, g} |

Regression of Metastasis Protocol

| Groups | LN foci | Lung foci |
|---|---|---|
| Buffer | 1.0 ± 0.3 ^{h} | 4.0 ± 1.1 ^{j, k} |
| T22-GFP-H6 | 1.1 ± 0.2 ⁱ | 1.7 ± 0.6 ^{l} |
| Free oligo-FdU | 0.8 ± 0.2 | 2.0 ± 0.7 ^{j, m} |
| T22-GFP-H6-FdU | 0.4 ± 0.1 ^{h, i} | 0.7 ± 0.4 ^{k, l, m} |

Mean + SE metastatic foci number per mouse counted in 3 randomly chosen histology sections
^{a} p = 0.04;
^{b} p = 0.01;
^{c} p = 0.001;
^{d} p = 0.002,
^{e} p = 0.006;
^{f} p = 0.002;
^{g} p = 0.006.
Mean + SE foci number per mouse counted in 6 randomly chosen histology sections
^{h} p = 0.03;
ⁱ p = 0.01;
^{j} p = 0.04;
^{k} p = 0.03;
^{l} p = 0.03,
^{m} p = 0.04
free-oligo-FdU: equimolecular doses of free oligo-FdU
See Table 2 for nanoconjugate-induced reduction in mean foci size.

Importantly, in contrast to T22-GFP-H6-FdU, free oligo-FdU did not reduce the total (FIG. 8b) or mean (Table 1) foci number at any site (LN, liver, lung, peritoneum), as compared to buffer-treated animals. Moreover, T22-GFP-H6-FdU induced also a 2.4 fold reduction in the size of peritoneal foci as compared to free oligo-FdU (p=0.01) (Table 2).

TABLE 2
Number of mice bearing metastatoic foci at the end of the pre-
vention of Metastasis experiment in T22-GFP-FdU, free oligo-
FdU o buffer-treated mice and size of the observed metatstic foci

| | Primary | | | | | | | | | |
|---|---|---|---|---|---|---|---|---|---|---|
| | tumor | | | | | | | | | |
| | Positive/ | | Metastasis | | | | | | | |
| Group | #Mice [%] | | Lymph nodes | | Liver | | Lung | | Peritoneal | |
| Buffer | 11/11 | [100] | 11/11 | [100] | 4/11 | [36] | 8/11 | [73]^{b} | 7/11 | [64]^{d} |
| Free oligo-FdU | 11/11 | [100] | 11/11 | [100] | 6/11 | [55]^{a} | 6/11 | [55]^{c} | 5/11 | [45] |
| T22-GFP-FdU | 12/12 | [100] | 9/12 | [75] | 2/12 | [17]^{a} | 2/12 | [17]^{b, c} | 2/12 | [17]^{d} |

| | Metastasis Size^{&} (μm² × 10⁻³) | | | |
|---|---|---|---|---|
| Buffer | 110.7 ± | 11.2 ± | 21.2 ± | 435.7 ± |
| | 15.5^{e} | 3.4 | 1.3^{f} | 67.2^{g} |
| Free oligo-FdU | 77.0 ± | 9.6 ± | 17.9 ± | 304.8 ± |
| | 14.2^{e} | 2.3 | 1.6 | 22.3^{h} |
| T22-GFP-FdU | 79.3 ± | 8.7 ± | 15.1 ± | 126.6 ± |
| | 11.1 | 2.6 | 2.2^{f} | 18.7^{g, h} |

* number of mice bearing a primary tumor out of the total mice per group
** number of mice affected by metastases out of the total number of mice per group
^{&}mean + SE metastic foci size
^{a}p = 0.05;
^{b}p = 0.012;
^{c}p = 0.05;
^{d}p = 0.036;
^{e}p = 0.009,
^{f}p = 0.032;
^{g}p = 0.002,
^{h}p = 0.01
See Table 1 for the reduction of total number of foci induced by T22-GFP-FdU, free olgigo-FdU or Buffer

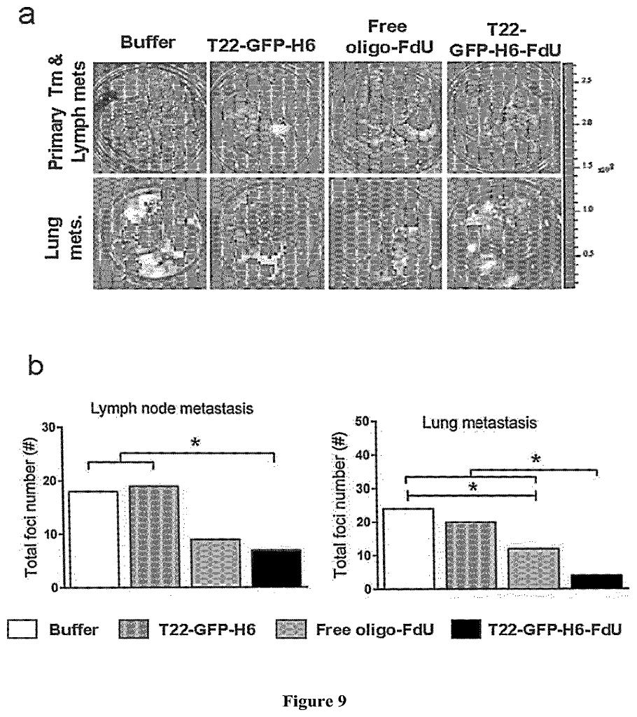

At the end of the regression of metastasis experiment, T22-GFP-H6-FdU-treated mice registered a lower number of lung metastasis, as measured by ex vivo luminescence emission (FIG. 9a) or a reduction in total lung foci count, in histology sections, than free-oligo-FdU (p=0.04), T22-GFP-H6 (p=0.03), or buffer (p=0.03) treated mice (FIG. 9b) Similar results were obtained when comparing the mean of lung foci number per mouse among groups (Table 1). T22-GFP-H6-FdU-treated mice registered also a significantly lower number of lymph node metastasis than buffer-treated (p=0.03); however, its effect was similar to that achieved by free oligo-FdU (FIG. 9b and Table 1).

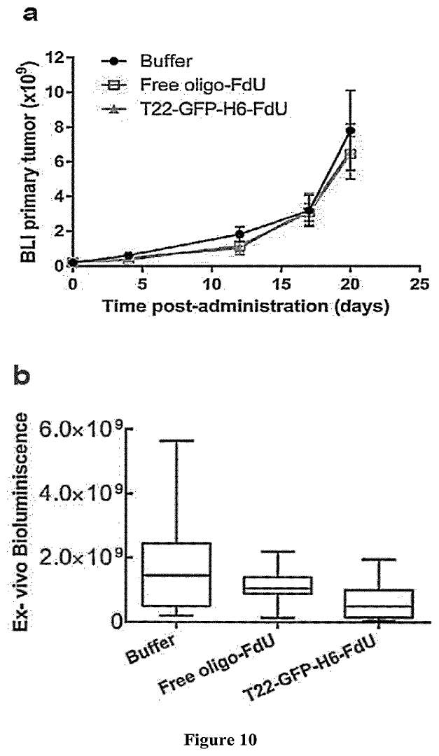

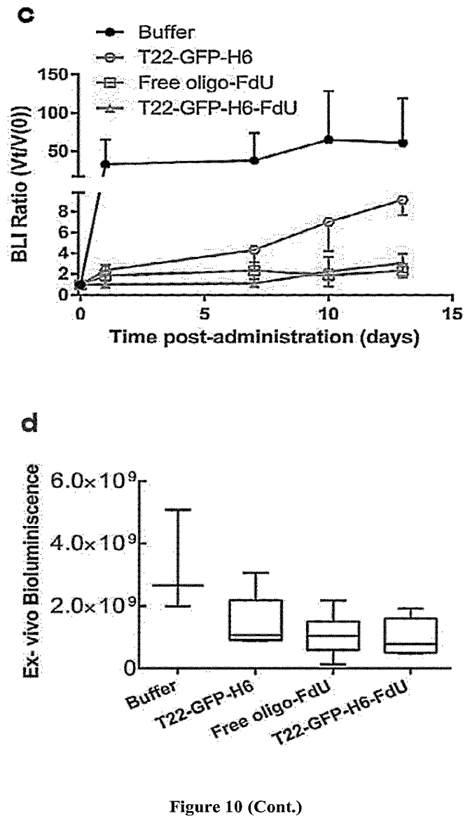

In summary, repeated T22-GFP-H6-FdU administration potently prevented metastasis development in peritoneum, liver and lung, whereas free oligo FdU did not prevent metastases at any site. In addition, T22-GFP-H6-FdU was more potent than free oligo-FdU in inducing the regression of established lung metastasis. Interestingly, both T22-GFP-H6-FdU and free oligo-FdU showed a similar inhibitory effect on primary tumor growth as measured by luminescence emission in vivo along time or ex vivo at the end of the experiment, in the prevention (FIG. 10a,b) or regression 193 (FIG. 10c,d) of metastasis experiments.

### Example 9: Site-Dependent CXCR4 Regulation, T22-GFP-H6-FdU CXCR4+ Cell Targeting and Antimetastatic Effect

Based on the clear site-dependent antimetastatic potency achieved by T22-GFP-H6-FdU in the prevention of metastasis experiment (FIG. 8b and Table 1), on its dependence on CXCR4 membrane expression for cell internalization (FIG. 2e) and on its capacity to selectively kill CXCR4⁺ cancer cells (FIG. 7a,b and FIG. 5a), the inventors investigated if CXCR4 expression registered after therapy correlated with the observed antimetastatic effect at the different sites. A site-dependent reduction in CXCR4⁺ CCF in metastatic foci at the end of the experiment was observed though detection with anti-CXCR4 IHC (as compared to basal levels), and nanoconjugate sensitivity, being higher in peritoneum, intermediate in liver and lung and non-existent in unresponsive lymph node metastases (FIG. 8c-d), which correlated with the antimetastatic effect at the different sites (FIG. 8b). In contrast to T22-GFP-H6-FdU-induced metastatic control, free oligo-FdU does not reduce CXCR4⁺ CCF (FIG. 8c) and seems to increase rather than decrease the number of metastases at the end of treatment in liver and peritoneum, as compared with buffer-treated mice (FIG. 8b and Table 1). Similarly, in the regression of metastasis experiment, the inventors observed a reduction in the CXCR4⁺ CCF in lung metastatic foci at the end of the experiment (FIG. 9c) and higher antimetastatic effect at this site (FIG. 9b) than in lymph node foci, which show no reduction in CXCR4″ CCF and poor response to T22-GFP-H6-FdU therapy (FIG. 9 and Table 1).

### Example 10: Lack of T22-GFP-H6-FdU Accumulation or Toxicity in Normal Tissues

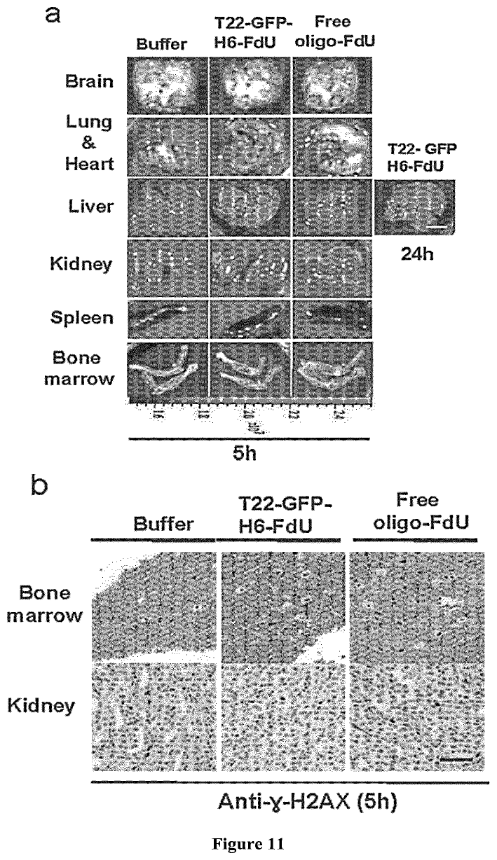

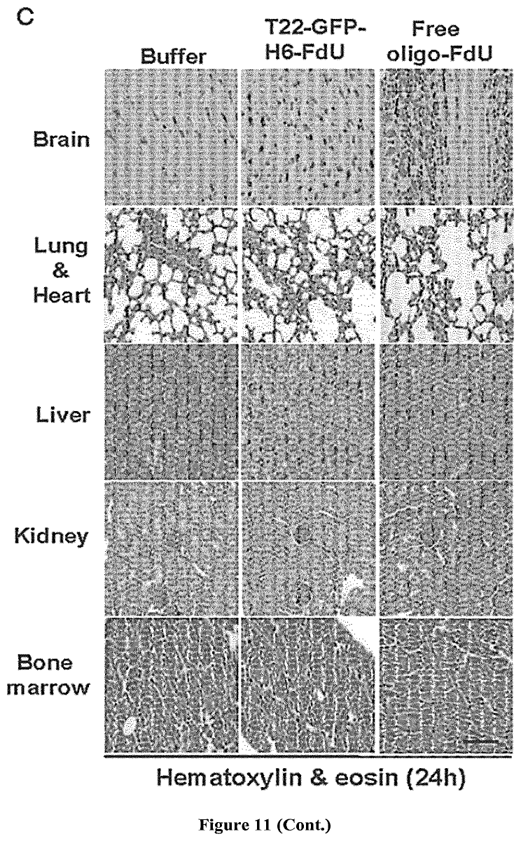

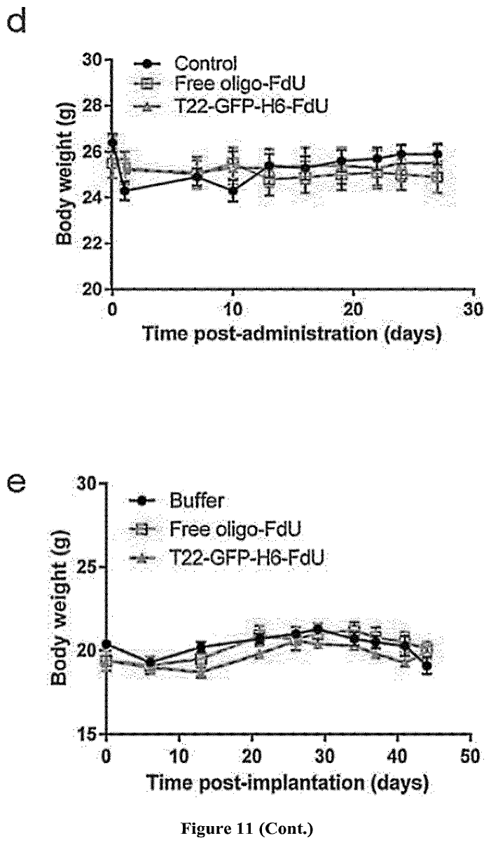

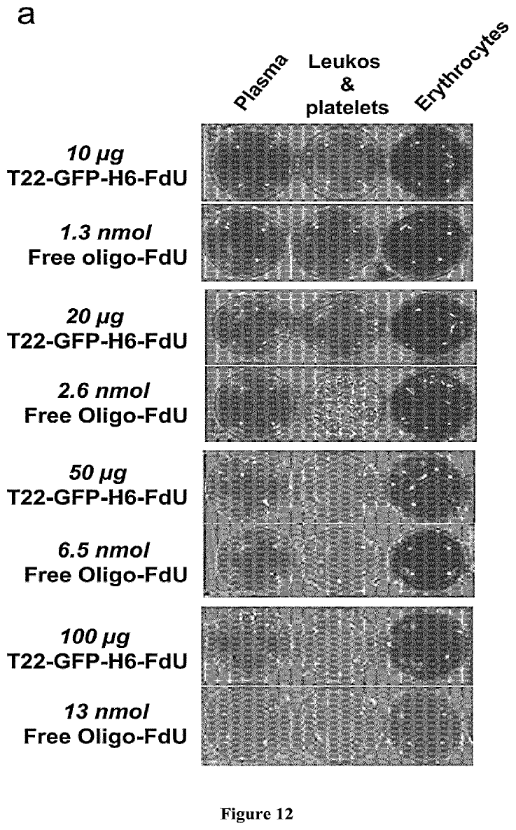

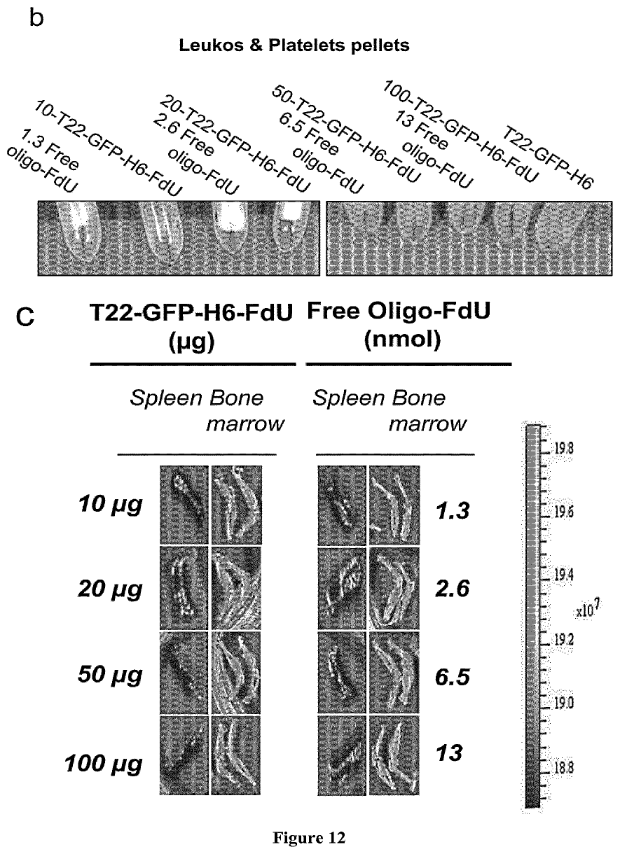

To estimate T22-GFP-H6-FdU therapeutic window, its biodistribution and induction of DNA damage or apoptosis in non-tumor tissues were analyzed. T22-GFP-H6-FdU injection lead to highly selective tumor tissue accumulation (FIG. 6b) as measured by fluorescence emission, whereas uptake in CXCR4 positive (bone marrow, spleen) or CXCR4 negative (kidney, lung, brain, heart or liver) normal tissues was undetectable, except for a transient accumulation in the liver (FIG. 11a), in the same experiment. Moreover, the level of DSBs, detected by anti-gH2AX IHC, in normal bone marrow 5 h after treatment was similar to that induced by free oligo-FdU (FIG. 11b), whereas it was undetectable in liver. DSB induction did not lead to apoptosis or histological alteration since they were not detected in bone marrow or liver 24 h post-administration (FIG. 11c). Therefore, consistently with the negligible nanoconjugate distribution to normal tissues, the lack of detectable apoptosis or histological alterations in all analyzed tissues, including bone marrow or circulating blood monocytes (FIG. 12), and the lack of mouse body weight loss in the regression (FIG. 11d) or prevention (FIG. 11e) of metastases experiments, or any sign of clinical toxicity indicate a wide therapeutic index for T22-GFP-H6-FdU at a dosage that achieves potent antimetastatic effect.

1-43. (canceled)

44. A method of treating a CXCR4⁺ cancer comprising administering a T22-nidogen-H6-[chemotherapeutic agent] composition in a nanoparticle dosage form to a subject in need thereof, wherein the administration of the composition to the subject results in a reduction of metastasis when compared to treating by administering the free chemotherapeutic agent to the subject.

45. The method of claim 44, wherein the nidogen is nidogen 1, nidogen 2, or a fragment or a mutant thereof.

46. The method of claim 44, wherein the chemotherapeutic agent is an anti-microtubule agent.

47. The method of claim 44, wherein the chemotherapeutic agent is selected from the group consisting of topoisomerase inhibitors, DNA alkylating agents, DNA strand break inducing agents, anti-metabolic agents, tyrosine kinase inhibitors, CDK inhibitors, MAP kinase inhibitors, EGFR inhibitors, VEGF inhibitors, anti-angiogenic polypeptides, and DNA cleaving agents.

48. The method of claim 44, wherein the cancer is selected from the group consisting of breast, heart, small intestine, colon, colorectal, spleen, kidney, bladder, head, neck, ovaries, prostate gland, brain, pancreatic, skin, bone, bone marrow, blood, thymus, womb, testicles, hepatobiliary system, and liver cancer.

49. The method of claim 44, wherein the cancer is a tumor.

50. The method of claim 49, wherein the tumor is selected from the group consisting of adenoma, angiosarcoma, astrocytoma, epithelial carcinoma, germinoma, glioblastoma, glioma, hemangioendothelioma, hemangiosarcoma, hematoma, hepatoblastoma, leukemia, lymphoma, medulloblastoma, melanoma, neuroblastoma, hepatobiliary cancer, osteosarcoma, retinoblastoma, rhabdomyosarcoma, sarcoma, and teratoma.

51. A method of treating a CXCR4⁺ cancer comprising administering a T22-nidogen-H6-[antibody] composition to a subject in need thereof, wherein the administration of the composition to the subject results in a reduction of metastasis when compared to treating by administering the free antibody to the subject.

52. The method of claim 51, wherein the antibody is selected from the group consisting of bevacizumab, trastuzumab, alemtuzumab, gemtuzumab, rituximab, tositumomab, MDX-210, oregovomab, edrecolomab, daclizumab, palivizumab, ibritumomab tiuxetan, cetuximab, MDX-447, MDX-22, MDX-220, I0R-C5, 10R-T6, IOR EGF/R3, celogovab, epratuzumab, pemtumomab, and gliomab-H.


SEQUENCE LISTING (52 sequences)

SEQ ID NO: 1 - protein, 9 aa, synthetic construct
  source 1..9: note = Polycationic peptide
RRRRRRRRR

SEQ ID NO: 2 - protein, 9 aa, synthetic construct
  source 1..9: note = Polycationic peptide
RRRGRGRRR

SEQ ID NO: 3 - protein, 9 aa, synthetic construct
  source 1..9: note = Polycationic peptide
RARGRGRRR

SEQ ID NO: 4 - protein, 9 aa, synthetic construct
  source 1..9: note = Polycationic peptide
RARGRGGGA

SEQ ID NO: 5 - protein, 18 aa, synthetic construct
  source 1..18: note = T22 peptide
RRWCYRKCYKGYCYRKCR

SEQ ID NO: 6 - protein, 21 aa, synthetic construct
  source 1..21: note = V1 peptide
LGASWHRPDKCCLGYQKRPLP

SEQ ID NO: 7 - protein, 67 aa, synthetic construct
  source 1..67: note = CXCL12 peptide
KPVSLSYRCPCRFFESHVARANVKHLKILNTPNCALQIVARLKNNNRQVCIDPKLKWIQEYLEKALN

SEQ ID NO: 8 - protein, 68 aa, synthetic construct
  source 1..68: note = vCCL2 peptide
LGASWHRPDKCCLGYQKRPLPQVLLSSWYPTSQLCSKPGVIFLTKRGRQVCADKDWVKKLMQQLPVTA

SEQ ID NO: 9 - protein, 14 aa, synthetic construct
  SITE 3: note = Xaa is L-3-(2-naphthyl)alanine
  SITE 8: note = Xaa is D-Lys
  SITE 12: note = Xaa is L-Citrulline
  source 1..14: note = T140 peptide
RRXCYRKXPYRXCR

SEQ ID NO: 10 - protein, 14 aa, synthetic construct
  SITE 3: note = Xaa is L-3-(2-naphthyl)alanine
  SITE 6: note = Xaa is L-Citrulline
  SITE 8: note = Xaa is D-Lys
  SITE 12: note = Xaa is L-Citrulline
  source 1..14: note = TN14003 peptide
RRXCYXKXPYRXCR

SEQ ID NO: 11 - protein, 14 aa, synthetic construct
  SITE 3: note = Xaa is L-3-(2-naphthyl)alanine
  SITE 8: note = Xaa is D-Citrulline
  SITE 12: note = Xaa is L-Citrullin
  source 1..14: note = TC 14012 peptide
RRXCYEKXPYRXCR

SEQ ID NO: 12 - protein, 14 aa, synthetic construct
  SITE 3: note = Xaa is L-3-(2-naphthyl)alanine
  SITE 6: note = Xaa is L-Citrulline
  SITE 8: note = Xaa is D-Glutamic acid
  SITE 12: note = Xaa is L-Citrulline
  source 1..14: note = TE14011 peptide
RRXCYXKXPYRXCR

SEQ ID NO: 13 - protein, 14 aa, synthetic construct
  SITE 3: note = Xaa is L-3-(2)-naphthyl)alanine
  SITE 6: note = Xaa is L-Citrulline
  SITE 8: note = Xaa is D-Lysine
  SITE 12: note = Xaa is L-Citrulline
  source 1..14: note = TZ14011 peptide
RRXCYXKXPYRXCR

SEQ ID NO: 14 - protein, 20 aa, synthetic construct
  source 1..20: note = GW-H1 peptide
GYNYAKKLANLAKKFANALW

SEQ ID NO: 15 - protein, 13 aa, synthetic construct
  source 1..13: note = A5G27
RLVSYNGIIFFLK

SEQ ID NO: 16 - protein, 69 aa, synthetic construct
  source 1..69: note = FNI/II/V
WQPPRARITGYIIKYEKPGSPPREVVPRPRPGVTEATITGLEPGTEYTIYVIALKNNQKSEPLIGRKKT

SEQ ID NO: 17 - protein, 18 aa, synthetic construct
  source 1..18: note = Seq-1-7 peptide
KYLAYPDSVHIWRKRKRK

SEQ ID NO: 18 - protein, 19 aa, synthetic construct
  source 1..19: note = Seq-1-8 peptide
KYLAYPDSVHIWRKRKRKR

SEQ ID NO: 19 - protein, 22 aa, synthetic construct
  source 1..22: note = Angiopep-2-7 peptide
TFFYGGSRGKRNNFKTEEYRKR

SEQ ID NO: 20 - protein, 31 aa, synthetic construct
  source 1..31: note = Nucleolin binding peptide
KDEPQRRSARLSAKPAPPKPEPKPKKAPAKK

SEQ ID NO: 21 - protein, 39 aa, synthetic construct
  source 1..39: note = Nucleolin binding peptide
KDEPQRRSARLSAKPAPPKPEPKPKKAPAKKRKRKRKRK

SEQ ID NO: 22 - protein, 14 aa, synthetic construct
  source 1..14: note = Linker/spacer
SGGTSGSTSGTGST

SEQ ID NO: 23 - protein, 15 aa, synthetic construct
  source 1..15: note = Linker/spacer
AGSSTGSSTGPGSTT

SEQ ID NO: 24 - protein, 7 aa, synthetic construct
  source 1..7: note = Linker/spacer
GGSGGAP

SEQ ID NO: 25 - protein, 8 aa, synthetic construct
  source 1..8: note = Linker/spacer
GGGVEGGG

SEQ ID NO: 26 - protein, 10 aa, synthetic construct
  source 1..10: note = 10 amino acid residues of the upper hinge region of murine IgG3
PKPSTPPGSS

SEQ ID NO: 27 - protein, 11 aa, synthetic construct
  source 1..11: note = Linker/spacer
APAETKAEPMT

SEQ ID NO: 28 - protein, 5 aa, synthetic construct
  source 1..5: note = Cleavage site
DDDDK

SEQ ID NO: 29 - protein, 5 aa, synthetic construct
  source 1..5: note = Cleavage site
IEDGR

SEQ ID NO: 30 - protein, 6 aa, synthetic construct
  source 1..6: note = Cleavage site
LVPRGS

SEQ ID NO: 31 - protein, 7 aa, synthetic construct
  source 1..7: note = Cleavage site
ENLYFQG

SEQ ID NO: 32 - protein, 8 aa, synthetic construct
  source 1..8: note = Cleavage site
LEVLFQGP

SEQ ID NO: 33 - protein, 7 aa, synthetic construct
  source 1..7: note = linker
GGSSRSS

SEQ ID NO: 34 - protein, 5 aa, synthetic construct
  source 1..5: note = linker
GGGNS

SEQ ID NO: 35 - protein, 238 aa, Aequorea victoria
MSKGEELFTGVVPILVELDGDVNGHKFSVSGEGEGDATYGKLTLKFICTTGKLPVPWPTLVTTFSYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYK

SEQ ID NO: 36 - protein, 609 aa, Homo sapiens [sequence omitted]

SEQ ID NO: 37 - protein, 1247 aa, Homo sapiens
MLASSSRIRAAWTRALLLPLLLAGPVGCLSRQELFPFGPGQGDLELEDGDDFVSPALELSGALRFYDRSDIDAVYVTTNGIIATSEPPAKESHPGLFPPTFGAVAPFLADLDTTDGLGKVYYREDLSPSITQRAAECVHRGFPEISFQPSSAVVVTWESVAPYQGPSRDPDQKGKRNTFQAVLASSDSSSYAIFLYPEDGLQFHTTFSKKENNQVPAVVAFSQGSVGFLWKSNGAYNIFANDRESVENLAKSSNSGQQGVWVFEIGSPATTNGVVPADVILGTEDGAEYDDEDEDYDLATTRLGLEDVGTTPFSYKALRRGGADTYSVPSVLSPRRAATERPLGPPTERTRSFQLAVETFHQQHPQVIDVDEVEETGVVFSYNTDSRQTCANNRHQCSVHAECRDYATGFCCSCVAGYTGNGRQCVAEGSPQRVNGKVKGRIFVGSSQVPIVFENTDLHSYVVMNHGRSYTAISTIPETVGYSLLPLAPVGGIIGWMFAVEQDGFKNGFSITGGEFTRQAEVTFVGHPGNLVIKQRFSGIDEHGHLTIDTELEGRVPQIPFGSSVHIEPYTELYHYSTSVITSSSTREYTVTEPERDGASPSRIYTYQWRQTITFQECVHDDSRPALPSTQQLSVDSVFVLYNQEEKILRYALSNSIGPVREGSPDALQNPCYIGTHGCDTNAACRPGPRTQFTCECSIGFRGDGRTCYDIDECSEQPSVCGSHTICNNHPGTFRCECVEGYQFSDEGTCVAVVDQRPINYCETGLHNCDIPQRAQCIYTGGSSYTCSCLPGFSGDGQACQDVDECQPSRCHPDAFCYNTPGSFTCQCKPGYQGDGFRCVPGEVEKTRCQHEREHILGAAGATDPQRPIPPGLFVPECDAHGHYAPTQCHGSTGYCWCVDRDGREVEGTRTRPGMTPPCLSTVAPPIHQGPAVPTAVIPLPPGTHLLFAQTGKIERLPLEGNTMRKTEAKAFLHVPAKVIIGLAFDCVDKMVYWTDITEPSIGRASLHGGEPTTIIRQDLGSPEGIAVDHLGRNIFWTDSNLDRIEVAKLDGTQRRVLFETDLVNPRGIVTDSVRGNLYWTDWNRDNPKIETSYMDGTNRRILVQDDLGLPNGLTFDAFSSQLCWVDAGTNRAECLNPSQPSRRKALEGLQYPFAVTSYGKNLYFTDWKMNSVVALDLAISKETDAFQPHKQTRLYGITTALSQCPQGHNYCSVNNGGCTHLCLATPGSRTCRCPDNTLGVDCIEQK

SEQ ID NO: 38 - protein, 1375 aa, Homo sapiens
MEGDRVAGRPVLSSLPVLLLLPLLMLRAAALHPDELFPHGESWGDQLLQEGDDESSAVVKLANPLHFYEARFSNLYVGTNGIISTQDFPRETQYVDYDFPTDFPAIAPFLADIDTSHGRGRVLYREDTSPAVLGLAARYVRAGFPRSARFTPTHAFLATWEQVGAYEEVKRGALPSGELNTFQAVLASDGSDSYALFLYPANGLQFLGTRPKESYNVQLQLPARVGFCRGEADDLKSEGPYFSLTSTEQSVKNLYQLSNLGIPGVWAFHIGSTSPLDNVRPAAVGDLSAAHSSVPLGRSFSHATALESDYNEDNLDYYDVNEEEAEYLPGEPEEALNGHSSIDVSFQSKVDTKPLEESSTLDPHTKEGTSLGEVGGPDLKGQVEPWDERETRSPAPPEVDRDSLAPSWETPPPYPENGSIQPYPDGGPVPSEMDVPPAHPEEEIVLRSYPASGHTTPLSRGTYEVGLEDNIGSNTEVFTYNAANKETCEHNHRQCSRHAFCTDYATGFCCHCQSKFYGNGKHCLPEGAPHRVNGKVSGHLHVGHTPVHFTDVDLHAYIVGNDGRAYTAISHIPQPAAQALLPLTPIGGLFGWLFALEKPGSENGFSLAGAAFTHDMEVTFYPGEETVRITQTAEGLDPENYLSIKTNIQGQVPYVSANFTAHISPYKELYHYSDSTVTSTSSRDYSLTFGAINQTWSYRIHQNITYQVCRHAPRHPSFPTTQQLNVDRVFALYNDEERVLRFAVTNQIGPVKEDSDPTPGNPCYDGSHMCDTTARCHPGTGVDYTCECASGYQGDGRNCVDENECATGFHRCGPNSVCINLPGSYRCECRSGYEFADDRHTCILITPPANPCEDGSHTCAPAGQARCVHHGGSTFSCACLPGYAGDGHQCTDVDECSENRCHPAATCYNTPGSFSCRCQPGYYGDGFQCIPDSTSSLTPCEQQQRHAQAQYAYPGARFHIPQCDEQGNFLPLQCHGSTGFCWCVDPDGHEVPGTQTPPGSTPPHCGPSPEPTQRPPTICERWRENLLEHYGGTPRDDQYVPQCDDLGHFIPLQCHGKSDFCWCVDKDGREVQGTRSQPGTTPACIPTVAPPMVRPTPRPDVTPPSVGTFLLYTQGQQIGYLPLNGTRLQKDAAKTLLSLHGSIIVGIDYDCRERMVYWTDVAGRTISRAGLELGAEPETIVNSGLISPEGLAIDHIRRTMYWTDSVLDKIESALLDGSERKVLFYTDLVNPRAIAVDPIRGNLYWTDWNREAPKIETSSLDGENRRILINTDIGLPNGLTFDPFSKLLCWADAGTKKLECTLPDGTGRRVIQNNLKYPFSIVSYADHFYHTDWRRDGVVSVNKHSGQFTDEYLPEQRSHLYGITAVYPYCPTGRK

SEQ ID NO: 39 - protein, 165 aa, Homo sapiens
MEMFQGLLLLLLLSMGGTWASKEPLRPRCRPINATLAVEKEGCPVCITVNTTICAGYCPTMTRVLQGVLPALPQVVCNYRDVRFESIRLPGCPRGVNPVVSYAVALSCQCALCRRSTTDCGGPKDHPLTCDDPRFQDSSSSKAPPPSLPSPSRLPGPSDTPILPQ

SEQ ID NO: 40 - protein, 98 aa, Homo sapiens
MIPGGLSEAKPATPEIQEIVDKVKPQLEEKTNETYGKLEAVQYKTQVVAGTNYYIKVRAGDNKYMHLKVFKSLPGQNEDLVLTGYQVDKNKDDELTGF

SEQ ID NO: 41 - protein, 210 aa, Homo sapiens
MASGQGPGPPRQECGEPALPSASEEQVAQDTEEVFRSYVFYRHQQEQEAEGVAAPADPEMVTLPLQPSSTMGQVGRQLAIIGDDINRRYDSEFQTMLQHLQPTAENAYEYFTKIATSLFESGINWGRVVALLGFGYRLALHVYQHGLTGFLGQVTRFVVDFMLHHCIARWIAQRGGWVAALNLGNGPILNVLVVLGVVLLGQFVVRRFFK

SEQ ID NO: 42 - protein, 15 aa, synthetic construct
  source 1..15: note = BH3 domain
VGRQLAIIGDDINRR

SEQ ID NO: 43 - protein, 193 aa, Homo sapiens
MARARQEGSSPEPVEGLARDGPRPFPLGRLVPSAVSCGLCEPGLAAAPAAPTLLPAAYLCAPTAPPAVTAALGGSRWPGGPRSRPRGPRPDGPQPSLSLAEQHLESPVPSAPGALAGGPTQAAPGVRGEEEQWAREIGAQLRRMADDLNAQYERRRQEEQQRHRPSPWRVLYNLIMGLLPLPRGHRAPEMEPN

SEQ ID NO: 44 - protein, 187 aa, Corynebacterium diphtheriae [sequence omitted]

SEQ ID NO: 45 - protein, 209 aa, Pseudomonas aeruginosa
GDISFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTGLTLAAPEAAGEVERLIGHPLPLRLDAITGPEEEGGRLETILGWPLAERTVVIPSAIPTDPRNVGGDLDPSSIPDKEQAISALPDYASQPGKPPREDLK

SEQ ID NO: 46 - protein, 5 aa, synthetic construct
  source 1..5: note = linker peptide
AAALE

SEQ ID NO: 47 - protein, 98 aa, Homo sapiens
MIPWGLSEAKPATPEIQEIVDKVKPQLEEKTNETYGKLEAVQYKTQVDAGTNYYIKVRAGDNKYMHLKVFNGPPGQNEDLVLTGYQVDKNKDDELTGF

SEQ ID NO: 48 - protein, 98 aa, Homo sapiens
MIPRGLSEAKPATPEIQEIVDKVKPQLEEKTNETYGKLEAVQYKTQVLASTNYYIKVRAGDNKYMHLKVFNGPPGQNEDLVRSGYQVDKNKDDELTGF

SEQ ID NO: 49 - protein, 98 aa, Homo sapiens
MIPRGLSEAKPATPEIQEIVDKVKPQLEEKTNETYGKLEAVQYKTQVVAGTNYYIKVRAGDNKYMHLKVFKSLPGQNEDLVLTGYQVDKNKDDELTGF

SEQ ID NO: 50 - protein, 98 aa, Homo sapiens
MIPGGLSEAKPATPEIQEIVDKVKPQLEEKTNETYGKLEAVQYKTQVLASTNYYIKVRAGDNKYMHLKVFKSLPGQNEDLVLTGYQVDKNKDDELTGF

SEQ ID NO: 51 - protein, 98 aa, Homo sapiens
MIPGGLSEAKPATPEIQEIVDKVKPQLEEKTNETYGKLEAVQYKTQVVAGTNYYIKVRAGDNKYMHLKVFNGPPGQNEDLVRSGYQVDKNKDDELTGF

SEQ ID NO: 52 - protein, 98 aa, Homo sapiens
MIPGGLSEAKPATPEIQEIVDKVKPQLEEKTNETYGKLEAVQYKTQVLASTNYYIKVRAGDNKYMHLKVFNGPPGQNEDLVRSGYQVDKNKDDELTGF